(19) United States
(12) Patent Application Publication
(10) Pub. No.: US 2022/0227735 A1
(43) Pub. Date: Jul. 21, 2022

(54) THERAPEUTIC METHODS AND COMPOUNDS

(71) Applicant: RUTGERS, THE STATE UNIVERSITY OF NEW JERSEY, New Brunswick, NJ (US)

(72) Inventors: Purnima BHANOT, New Brunswick, NJ (US); David J. AUGERI, New Brunswick, NJ (US); John A. GILLERAN, New Brunswick, NJ (US); Raheel FONDEKAR, New Brunswick, NJ (US)

(73) Assignee: RUTGERS, THE STATE UNIVERSITY OF NEW JERSEY, New Brunswick, NJ (US)

(21) Appl. No.: 17/605,732

(22) PCT Filed: Apr. 22, 2020
(86) PCT No.: PCT/US2020/029385

§ 371 Date: Oct. 22, 2021

Related U.S. Application Data
(60) Provisional application No. 62/839,503, filed on Apr. 26, 2019.

(51) Int. Cl. C07D 401/14; C07D 409/14; C07D 471/04

(52) U.S. Cl. CPC C07D 401/14; C07D 471/04; C07D 409/14

(57) ABSTRACT

The invention provides a compound of formula I: (I) or a pharmaceutically acceptable salt thereof, wherein R¹-R⁵ Y have any of the values described in the specification, as well as compositions comprising a compound of formula I. The compounds are useful to treat malaria.

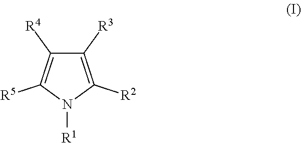

### PRIORITY OF INVENTION

This application claims priority to U.S. Provisional Application No. 62/839,503, filed 26 Apr. 2019. The entire content of this application is incorporated herein by reference.

### STATEMENT REGARDING FEDERALLY SPONSORED RESEARCH

This invention was made with government support under AI128052 awarded by the National Institutes of Health. The Government has certain rights in this invention.

### BACKGROUND OF THE INVENTION

Malaria begins when the bite of an infected mosquito introduces parasite stages termed ‘sporozoites’ into humans. Sporozoites infect hepatocytes, differentiate and divide within them to form ‘liver stages.’ Mature liver stages exit the infected hepatocyte and initiate invasion of RBCs giving rise to the symptomatic erythrocytic cycle that consists of repeated rounds of RBC invasion. Some erythrocytic stage parasites form dormant sexual stages termed ‘gametocytes,’ which are required for transmission of parasites from humans to mosquitoes.

The emergence and spread of artemisinin-resistant P. falciparum in Southeast Asia is a compelling reminder of the ongoing need to identify and pharmacologically validate new targets for malaria prophylaxis and treatment (see Ashley E A, et al., The New England Journal of Medicine, 2014, 371, 5, 411-23; Miotto O, et al., Nat Genet., 2013, 45, 6, 48-55; Takala-Harrison S, et al., J Infect Dis., 2015, 211, 5, 670-9; and Wells T N, et al., Nature Reviews Drug Discovery, 2015, 14, 6, 424-42). Drugs that target pre-erythrocytic stages (sporozoites and liver stages) are an essential component of the anti-malarial effort, because a decrease in liver infection by sporozoites significantly reduces severity and incidence of malaria (see Alonso P L, et al., Lancet, 2005, 366, 9502, 2012-8). Additionally, drugs that block sporozoite infection could reduce P. vivax hypnozoite formation, the major cause of disease relapse, and the number of asymptomatic individuals who serve as reservoirs of infection and transmission (see Walldorf J A, et al., PloS one, 2015, 10, 7, e0134061). Finally, even if all symptomatic malaria cases could be treated, the global eradication of malaria requires the prevention of new infections.

Prevention of new infections is beneficial in many scenarios—for malaria control in areas with seasonal transmission, for stopping the spread of a local outbreak and for protecting non-immune travelers to endemic regions or individuals with reduced immunity living in once-endemic regions. New infections can be prevented through the use of a vaccine but currently, there is no commercially available malaria vaccine and the most advanced vaccine candidate, RTSS/AS01 is only partially effective (see Olotu A, et al., The New England Journal of Medicine, 2016, 374, 26, 2519-29). In the absence of a vaccine, malaria control has relied on chemoprotection. The utility of chemoprotection is highlighted by the marked reduction in clinical and severe malaria in children who were administered monthly chemoprotection during the transmission season in the Sahel region of Africa (see Meremikwu M M. et al., The Cochrane Database of Systematic Reviews, 2012, 2, CD003756). The combined use of chemoprotective drugs, bed nets, insecticide spraying and a partially protective vaccine can achieve effective and sustainable malaria control.

Causal chemoprotection can be achieved by targeting the parasite's pre-erythrocytic stages. A cocktail of drugs that prevent sporozoite infection and liver stage development could prevent infection, and suppress drug resistance. Strategic use of these chemoprotective drug combinations could leverage the effect of a partially effective vaccine, bed nets and indoor residual spraying, all of which target sporozoite infection of the liver, to achieve effective and sustainable malaria control and elimination.

There are few adequate methods for inhibiting Plasmodium Falciparum Protein Kinase G that lack toxicity and undesired side effects. Chemical approaches with marginal treatment options presents the patient with healthcare challenges. There are several chemotypes for targeting the Plasmodium falciparum kinase G(PfPKG) discussed in patent applications (see U.S. Pat. No. 5,792,778). Also, there are published articles that focus on the development of PKG inhibitors (see Tsagris, D. J., et al., Bioorganic & Medicinal Chemistry Letters. 2005, 28, 19, 3168-3173; and Baker, D. A., et al, Nature Commnunications, 2017, 8, 1, 430), as well as inhibitors of other targets such as plasmodium kinases PI4K (see Kandepedu, Nishanth, et al., Journal of Medicinal Chemistry. 2018, 61(13) 5692-5703; Dembele, L., et al., Scientific Reports, 2018, 7, 1, 2325; and Paquet, T., et al, Science Translational Medicine, 2017, 9, 387, 9735) and FIKK kinase (see Lin, B. C., et al., Bioorganic Chemistry, 2017, 75, 217-223). In addition, the plasmodium form of thymidylate kinase has been targeted for malaria (see Whittingham, J. L., et al. Biochemical Journal, 428, 3, 499-509).

In 2005, Merck published a research article on trisubstituted pyrroles as potent and broad spectrum anticoccidial agents through inhibition of a novel cGMP-dependent protein kinase G (PKG) (see Biftu, T., et al., Bioorganic & Medicinal Chemistry Letters. 2005, 15, 13, 3296-3301). Subsequent articles were published that included more highly developed compounds as broad spectrum anticoccidial agents (see Liang, G. B., et al. Bioorganic & Medicinal Chemistry Letters, 2005, 18, 6, 2019-2022).

In spite of the above referenced materials, existing anti-malarials that target pre-eythrocytic stages and/or hypnozoites—primaquine, tafenoquine and atovaquone, have significant side-effects or are expensive. Accordingly, there is a need for novel drugs that target pre-erythrocytic stages.

### SUMMARY OF THE INVENTION

Inhibiting P. falciparum cGMP dependent protein kinase (PfPKG) blocks the pre-erythrocytic cycle. Data strongly suggests that (1) PfPKG is essential for sporozoite infection of hepatocytes and the parasite's intrahepatic development, (2) it is chemically targetable, and (3) its chemical inhibition prevents sporozoite infection and significantly delays the appearance of pathology-causing erythrocytic stages in mouse models of malaria infection. The invention provides compounds and methods for the targeted inhibition of the development of PfPKG. In particular, invention provides compounds and methods that target pre-erythrocytic stages of malaria. The methods and compounds of the invention are useful for treating malaria and for preventing malaria.

In one aspect the present invention provides a compound of formula I:

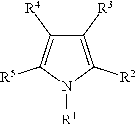

wherein:

R¹ is H or (C₁-C₃)alkyl;

R² is a 5-10 membered monocyclic or bicyclic heterocyclic ring comprising 1 or 2 nitrogen atoms, which 5-10 membered monocyclic or bicyclic heterocyclic ring is optionally substituted with 1, 2, 3, 4, or 5 substituents independently selected from the group consisting of (C₁-C₆)alkyl; and R³ is H, halo, hydroxy, cyano, NR^{a}R^{b}, —C(═O)NR^{a}R^{b}, (C₁-C₆)alkoxycarbonyl, (C₂-C₆)alkenyl, (C₂-C₆)alkynyl or (C₁-C₆)alkyl that is optionally substituted with one or more substituents independently selected from the group consisting of halo, hydroxy, cyano, NR^{a}R^{b}, (C₁-C₆)alkoxycarbonyl, —S(═O)₂—R^{e} and —C(═O)NR^{a}R^{b},

or

R² is a 5-10 membered monocyclic or bicyclic heterocyclic ring comprising 1 or 2 nitrogen atoms, which 5-10 membered monocyclic or bicyclic heterocyclic ring is optionally substituted with one or more (e.g., 1, 2, 3, 4, or 5) substituents R^{x}, wherein a carbon atom of the R² 5-10 membered monocyclic or bicyclic heterocyclic ring adjacent to the position that attaches R² to the remainder of formula I, together with R³ forms a fused phenyl ring:

R⁴ is a tetrazolo[1,5-a]pyridine ring or a pyridine ring, which tetrazolo[1,5-a]pyridine ring or a pyridine ring is optionally substituted with 1, 2, 3, or 4 substituents independently selected from the group consisting of halo, hydroxy, cyano, NR^{c}R^{d}, —C(═O)NR^{c}R^{d}, (C₁-C₆)alkoxycarbonyl, (C₂-C₆)alkenyl, (C₂-C₆)alkynyl, and (C₁-C₆)alkyl that is optionally substituted with one or more substituents independently selected from the group consisting of halo, hydroxy, cyano, NR^{c}R^{d}, (C₁-C₆)alkoxycarbonyl, and —C(═O)NR^{c}R^{d};

R⁵ is phenyl or thiophene, which phenyl or thiophene is optionally substituted with one or more substituents independently selected from the group consisting of halo, hydroxy, cyano, NR^{f}R^{g}, —C(═O)NR^{f}R^{g}, (C₁-C₆)alkoxycarbonyl, (C₁-C₆)alkoxy, (C₂-C₆)alkenyl. (C₂-C₆)alkynyl, thiophene, and (C₁-C₆)alkyl that is optionally substituted with one or more substituents independently selected from the group consisting of halo, hydroxy, cyano, NR^{f}R^{g}, (C₁-C₆)alkoxycarbonyl, and —C(═O)NR^{f}R^{g};

each R^{a} and R^{b} is independently selected from the group consisting of H, (C₁-C₆)alkyl, (C₃-C₆)cycloalkyl, and (C₃-C₆)cycloalkyl(C₁-C₆)alkyl; or R^{c} and R^{d} together with the nitrogen to which they are attached form a aziridino, azetidino, morpholino, piperazino, pyrrolidino or piperidino;

each R^{c} and R^{d} is independently selected from the group consisting of H, (C₁-C₆)alkyl, (C₃-C₆)cycloalkyl, and (C₃-C₆)cycloalkyl(C₁-C₆)alkyl; or R^{c} and R^{d} together with the nitrogen to which they are attached form a aziridino, azetidino, morpholino, piperazino, pyrrolidino or piperidino;

R^{e} is (C₁-C₆)alkyl;

each R^{f} and R^{g} is independently selected from the group consisting of H, (C₁-C₆)alkyl, (C₃-C₆)cycloalkyl, and (C₃-C₆)cycloalkyl(C₁-C₆)alkyl; or R^{f} and R^{g} together with the nitrogen to which they are attached form a aziridino, azetidino, morpholino, piperazino, pyrrolidino or piperidino; and

each R^{x} is independently selected from the group consisting of (C₁-C₆)alkyl, (C₂-C₆)alkenyl, (C₂-C₆)alkynyl, aryl, aryl(C₁-C₆)alkyl, (C₃-C₆)cycloalkyl, (C₁-C₆)alkoxycarbonyl, (C₁-C₆)alkanoyloxy, and (C₃-C₆)cycloalkyl(C₁-C₆)alkyl, wherein each R¹ is optionally substituted with one or more groups independently selected from halo, cyano, nitro, (C₁-C₆)alkyl, and (C₁-C₆)alkoxy;

or a pharmaceutically acceptable salt thereof;

for use in medical therapy (e.g. anti-malarial therapy).

In one aspect the present invention provides a method for reducing a human's susceptibility to malaria comprising administering to the human, a compound of formula I or a pharmaceutically acceptable salt thereof.

In one aspect the present invention provides a method for preventing malaria in a human comprising administering to the human, a compound of formula I or a pharmaceutically acceptable salt thereof.

The invention also provides a pharmaceutical composition comprising a compound of formula I or a pharmaceutically acceptable salt thereof, and a pharmaceutically acceptable excipient.

The invention also provides a compound of formula I or a pharmaceutically acceptable salt thereof for the prophylactic or therapeutic treatment of malaria.

The invention also provides the use of a compound of formula I or a pharmaceutically acceptable salt thereof to prepare a medicament for preventing or treating malaria in a human.

The invention also provides a compound of formula I:

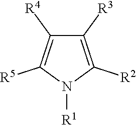

wherein:

R¹ is H or (C₁-C₃)alkyl;

R² is a 5-10 membered monocyclic or bicyclic heterocyclic ring comprising 1 or 2 nitrogen atoms, which 5-10 membered monocyclic or bicyclic heterocyclic ring is optionally substituted with 1, 2, 3, 4, or 5 substituents independently selected from the group consisting of (C₁-C₆)alkyl; and R³ is H, halo, hydroxy, cyano, NR^{a}R^{b}, —C(═O)NR^{a}R^{b}, (C₁-C₆)alkoxycarbonyl, (C₂-C₆)alkenyl, (C₂-C₆)alkynyl or (C₁-C₆)alkyl that is optionally substituted with one or more substituents independently selected from the group consisting of halo, hydroxy, cyano, NR^{a}R^{b}, (C₁-C₆)alkoxycarbonyl, —S(═O)₂—R^{e} and —C(═O)NR^{a}R^{b};

or

R² is a 5-10 membered monocyclic or bicyclic heterocyclic ring comprising 1 or 2 nitrogen atoms, which 5-10 membered monocyclic or bicyclic heterocyclic ring is optionally substituted with one or more (e.g., 1, 2, 3, 4, or 5) substituents R^{x}, wherein a carbon atom of the R² 5-10 membered monocyclic or bicyclic heterocyclic ring adjacent to the position that attaches R² to the remainder of formula I, together with R³ forms a fused phenyl ring;

R⁴ is a tetrazolo[1,5-a]pyridine ring or a pyridine ring, which tetrazolo[1,5-a]pyridine ring or a pyridine ring is optionally substituted with 1, 2, 3, or 4 substituents independently selected from the group consisting of halo, hydroxy, cyano, NR^{c}R^{d}, —C(═O)NR^{c}R^{d}. (C₁-C₆)alkoxycarbonyl, (C₂-C₆)alkenyl, (C₂-C₆)alkynyl, and (C₁-C₆)alkyl that is optionally substituted with one or more substituents independently selected from the group consisting of halo, hydroxy, cyano, NR^{c}R^{d}, (C₁-C₆)alkoxycarbonyl, and —C(═O)NR^{c}R^{d};

R⁵ is phenyl or thiophene, which phenyl or thiophene is optionally substituted with one or more substituents independently selected from the group consisting of halo, hydroxy, cyano, NR^{f}R^{g}, —C(═O)NR^{f}R^{g}, (C₁-C₆)alkoxycarbonyl, (C₁-C₆)alkoxy, (C₂-C₆)alkenyl, (C₂-C₆)alkynyl, and (C₁-C₆)alkyl that is optionally substituted with one or more substituents independently selected from the group consisting of halo, hydroxy, cyano, NR^{f}R^{g}, (C₁-C₆)alkoxycarbonyl, and —C(═O)NR^{f}R^{g};

each R^{a} and R^{b} is independently selected from the group consisting of H, (C₁-C₆)alkyl, (C₃-C₆)Cycloalkyl, and (C₃-C₆)cycloalkyl(C₁-C₆)alkyl; or R^{a} and R^{b} together with the nitrogen to which they are attached form a aziridino, azetidino, morpholino, piperazino, pyrrolidino or piperidino;

each R^{c} and R^{d} is independently selected from the group consisting of H, (C₁-C₆)alkyl, (C₃-C₆)cycloalkyl, and (C₃-C₆)cycloalkyl(C₁-C₆)alkyl; or R^{c} and R^{d} together with the nitrogen to which they are attached form a aziridino, azetidino, morpholino, piperazino, pyrrolidino or piperidino;

R^{e} is (C₁-C₆)alkyl;

each R^{f} and R^{g} is independently selected from the group consisting of H, (C₁-C₆)alkyl, (C₃-C₆)cycloalkyl, and (C₃-C₆)cycloalkyl(C₁-C₆)alkyl; or R^{f} and R^{g} together with the nitrogen to which they are attached form a aziridino, azetidino, morpholino, piperazino, pyrrolidino or piperidino; and

each R^{x} is independently selected from the group consisting of (C₁-C₆)alkyl, (C₂-C₆)alkenyl, (C₂-C₆)alkynyl, aryl, aryl(C₁-C₆)alkyl, (C₃-C₆)cycloalkyl, (C₁-C₆)alkoxycarbonyl, (C₁-C₆)alkanoyloxy, and (C₃-C₆)cycloalkyl(C₁-C₆)alkyl, wherein each R^{x} is optionally substituted with one or more groups independently selected from halo, cyano, nitro, (C₁-C₆)alkyl, and (C₁-C₆)alkoxy,

or a pharmaceutically acceptable salt thereof;

provided the compound is not:

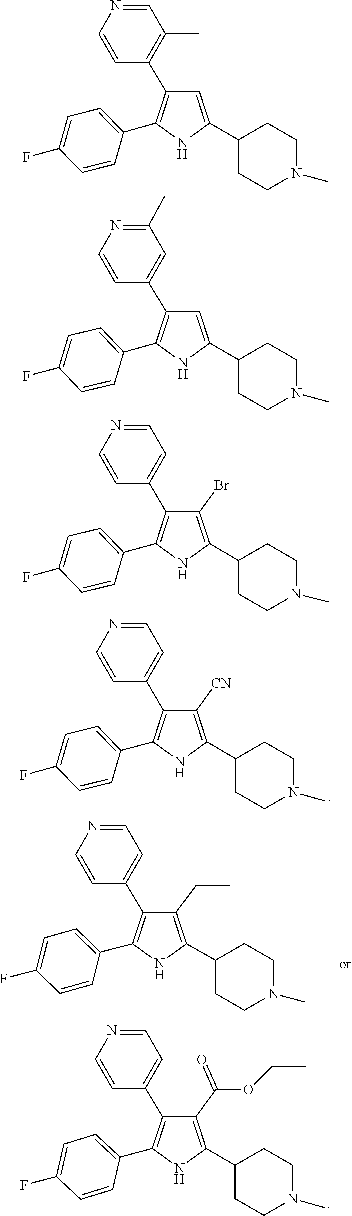

The invention also provides processes and intermediates disclosed herein that are useful for preparing a compound of formula I or a salt thereof.

### BRIEF DESCRIPTION OF THE DRAWINGS

FIG. 1 shows data for representative compounds of the invention from Example 38.

### DETAILED DESCRIPTION OF THE INVENTION

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight or branched chain hydrocarbon radical, having the number of carbon atoms designated (i.e., C₁₋₈ means one to eight carbons). Examples include (C₁-C₈)alkyl, (C₂-C₈)alkyl, C₁-C₆)alkyl, (C₂-C₆)alkyl and (C₃-C₆)alkyl. Examples of alkyl groups include methyl, ethyl, n-propyl, iso-propyl, n-butyl, t-butyl, iso-butyl, sec-butyl, n-pentyl, n-hexyl, n-heptyl, n-octyl, and and higher homologs and isomers.

The term “alkenyl” refers to an unsaturated alkyl radical having one or more double bonds. Examples of such unsaturated alkyl groups include vinyl, 2-propenyl, crotyl, 2-isopentenyl, 2-(butadienyl), 2,4-pentadienyl, 3-(1,4-pentadienyl) and the higher homologs and isomers.

The term “alkynyl” refers to an unsaturated alkyl radical having one or more triple bonds. Examples of such unsaturated alkyl groups ethynyl, 1- and 3-propynyl, 3-butynyl, and higher homologs and isomers.

The term “alkoxy” refers to an alkyl groups attached to the remainder of the molecule via an oxygen atom (“oxy”).

The term “cycloalkyl” refers to a saturated or partially unsaturated (non-aromatic) all carbon ring having 3 to 8 carbon atoms (i.e., (C₃-C₈)carbocycle). The term also includes multiple condensed, saturated all carbon ring systems (e.g., ring systems comprising 2, 3 or 4 carbocyclic rings). Accordingly, carbocycle includes multicyclic carbocyles such as a bicyclic carbocycles (e.g., bicyclic carbocycles having about 3 to 15 carbon atoms, about 6 to 15 carbon atoms, or 6 to 12 carbon atoms such as bicyclo[3.1.0]hexane and bicyclo[2.1.1]hexane), and polycyclic carbocycles (e.g tricyclic and tetracyclic carbocycles with up to about 20 carbon atoms). The rings of the multiple condensed ring system can be connected to each other via fused, spiro and bridged bonds when allowed by valency requirements. For example, multicyclic carbocyles can be connected to each other via a single carbon atom to form a spiro connection (e.g., spiropentane, spiro[4,5]decane, etc), via two adjacent carbon atoms to form a fused connection (e.g., carbocycles such as decahydronaphthalene, norsabinane, norcarane) or via two non-adjacent carbon atoms to form a bridged connection (e.g., norbornane, bicyclo[2.2.2]octane, etc). Non-limiting examples of cycloalkyls include cyclopropyl, cyclobutyl, cyclopentyl, cyclohexyl, bicyclo[2.2.1]heptane, pinane, and adamantane.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heterocycle” refers to a single saturated or partially unsaturated ring that has at least one atom other than carbon in the ring, wherein the atom is selected from the group consisting of oxygen, nitrogen and sulfur; the term also includes multiple condensed ring systems that have at least one such saturated or partially unsaturated ring, which multiple condensed ring systems are further described below. Thus, the term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The sulfur and nitrogen atoms may also be present in their oxidized forms. Exemplary heterocycles include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more groups selected from cycloalkyl, aryl, and heterocycle to form the multiple condensed ring system. The rings of the multiple condensed ring system can be connected to each other via fused, spiro and bridged bonds when allowed by valency requirements. It is to be understood that the individual rings of the multiple condensed ring system may be connected in any order relative to one another. It is also to be understood that the point of attachment of a multiple condensed ring system (as defined above for a heterocycle) can be at any position of the multiple condensed ring system including a heterocycle, aryl and carbocycle portion of the ring. In one embodiment the term heterocycle includes a 3-15 membered heterocycle. In one embodiment the term heterocycle includes a 3-10 membered heterocycle. In one embodiment the term heterocycle includes a 3-8 membered heterocycle. In one embodiment the term heterocycle includes a 3-7 membered heterocycle. In one embodiment the term heterocycle includes a 3-6 membered heterocycle. In one embodiment the term heterocycle includes a 4-6 membered heterocycle. In one embodiment the term heterocycle includes a 3-10 membered monocyclic or bicyclic heterocycle comprising 1 to 4 heteroatoms. In one embodiment the term heterocycle includes a 3-8 membered monocyclic or bicyclic heterocycle heterocycle comprising 1 to 3 heteroatoms. In one embodiment the term heterocycle includes a 3-6 membered monocyclic heterocycle comprising 1 to 2 heteroatoms. In one embodiment the term heterocycle includes a 4-6 membered monocyclic heterocycle comprising 1 to 2 heteroatoms. Exemplary heterocycles include, but are not limited to aziridinyl, azetidinyl, pyrrolidinyl, piperidinyl, homopiperidinyl, morpholinyl, thiomorpholnyl, piperaznyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, 1,4-benzodioxanyl, spiro[cyclopropane-1,1′-isoindolinyl]-3′-one, isoindolinyl-1-one, 2-oxa-6-azaspiro[3.3]heptanyl, imidazolidin-2-one imidazolidine, pyrazolidine, butyrolactam, valerolactam, imidazolidinone, hydantoin, dioxolane, phthalimide, and 1,4-dioxane.

The term “alkoxycarbonyl” as used herein refers to a group (alkyl)-O—C(═O)—, wherein the term alkyl has the meaning defined herein.

As used herein, the term “heteroatom” is meant to include oxygen (O), nitrogen (N), sulfur (S) and silicon (Si).

As used herein, the term “protecting group” refers to a substituent that is commonly employed to block or protect a particular functional group on a compound. For example, an “amino-protecting group” is a substituent attached to an amino group that blocks or protects the amino functionality in the compound. Suitable amino-protecting groups include acetyl, trifluoroacetyl, t-butoxycarbonyl (BOC), benzyloxycarbonyl (CBZ) and 9-fluorenylmethylenoxycarbonyl (Fmoc). Similarly, a “hydroxy-protecting group” refers to a substituent of a hydroxy group that blocks or protects the hydroxy functionality. Suitable protecting groups include acetyl and silyl. A “carboxy-protecting group” refers to a substituent of the carboxy group that blocks or protects the carboxy functionality. Common carboxy-protecting groups include phenylsulfonylethyl, cyanoethyl, 2-(trimethylsilyl)ethyl, 2-(trimethylsilyl)ethoxymethyl, 2-(p-toluenesulfonyl)ethyl, 2-(p-nitrophenylsulfenyl)ethyl, 2-(diphenylphosphino)-ethyl, nitroethyl and the like. For a general description of protecting groups and their use, see P. G. M. Wuts and T. W. Greene, Greene's Protective Groups in Organic Synthesis 4^{th} edition, Wiley-Interscience, New York, 2006.

As used herein a wavy line “ [symbol] ” that intersects a bond in a chemical structure indicates the point of attachment of the bond that the wavy bond intersects in the chemical structure to the remainder of a molecule.

The terms “treat”, “treatment”, or “treating” to the extent it relates to a disease or condition includes inhibiting the disease or condition, eliminating the disease or condition, and/or relieving one or more symptoms of the disease or condition. The terms “treat”. “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The compounds disclosed herein can also exist as tautomeric isomers in certain cases. Although only one delocalized resonance structure may be depicted, all such forms are contemplated within the scope of the invention.

It is understood by one skilled in the art that this invention also includes any compound claimed that may be enriched at any or all atoms above naturally occurring isotopic ratios with one or more isotopes such as, but not limited to, deuterium (²H or D). As a non-limiting example, a —CH₃ group may be substituted with —CD₃.

The pharmaceutical compositions of the invention can comprise one or more excipients. When used in combination with the pharmaceutical compositions of the invention the term “excipients” refers generally to an additional ingredient that is combined with the compound of formula (I) or the pharmaceutically acceptable salt thereof to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Stereochemical definitions and conventions used herein generally follow S. P. Parker, Ed., McGraw-Hill Dictionary of Chemical Terms (1984) McGraw-Hill Book Company, New York: and Eliel, E. and Wilen, S., “Stereochemistry of Organic Compounds”, John Wiley & Sons, Inc., New York, 1994. The compounds of the invention can contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L. or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer can also be referred to as an enantiomer, and a mixture of such isomers is often called an enantiomeric mixture. A 50:50 mixture of enantiomers is referred to as a racemic mixture or a racemate, which can occur where there has been no stereoselection or stereospecificity in a chemical reaction or process. The terms “racemic mixture” and “racemate” refer to an equimolar mixture of two enantiomeric species, devoid of optical activity.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

The term “residue” as it applies to the residue of a compound refers to a compound that has been modified in any manner which results in the creation of an open valence wherein the site of the open valence. The open valence can be created by the removal of 1 or more atoms from the compound (e.g., removal of a single atom such as hydrogen or removal of more than one atom such as a group of atoms including but not limited to an amine, hydroxyl, methyl, amide (e.g., —C(═O)NH₂) or acetyl group). The open valence can also be created by the chemical conversion of a first function group of the compound to a second functional group of the compound (e.g., reduction of a carbonyl group, replacement of a carbonyl group with an amine) followed by the removal of 1 or more atoms from the second functional group to create the open valence.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; and aryl can be phenyl, indenyl, or naphthyl.

In one aspect, R¹ is H and R⁵ is phenyl or thiophene, which phenyl or thiophene is optionally substituted with one or more substituents independently selected from the group consisting of halo, hydroxy, cyano, NR^{f}R^{g}, —C(═O)NR^{f}R^{g}, (C₁-C₆)alkoxycarbonyl, (C₁-C₆)alkoxy, (C₂-C₆)alkenyl, (C₂-C₆)alkynyl, and (C₁-C₆)alkyl that is optionally substituted with one or more substituents independently selected from the group consisting of halo, hydroxy, cyano, NR^{f}R^{g}, (C₁-C₆)alkoxycarbonyl, and —C(═O)NR^{f}R^{g}.

In one aspect, R² is a 5-10 membered monocyclic or bicyclic heterocyclic ring comprising 1 or 2 nitrogen atoms, which 5-10 membered monocyclic or bicyclic heterocyclic ring is optionally substituted with 1, 2, 3, 4, or 5 substituents independently selected from the group consisting of (C₁-C₆)alkyl; and R³ is halo, hydroxy, cyano, NR^{a}R^{b}, —C(═O)NR^{a}R^{b}, (C₁-C₆)alkoxycarbonyl, (C₂-C₆)alkenyl, (C₂-C₆)alkynyl or (C₁-C₆)alkyl that is optionally substituted with one or more substituents independently selected from the group consisting of halo, hydroxy, cyano, NR^{a}R^{b}, (C₁-C₆)alkoxycarbonyl, and —C(═O)NR^{a}R^{b}.

A specific value for R² is piperidinyl, 3-azabicyclo[3.1.0]hexanyl, or 8-azabicyclo[3.2.1]octanyl, which R² is optionally substituted with 1, 2, 3, 4, or 5 substituents independently selected from the group consisting of (C₁-C₆)alkyl.

A specific value for R² is selected from the group consisting of:

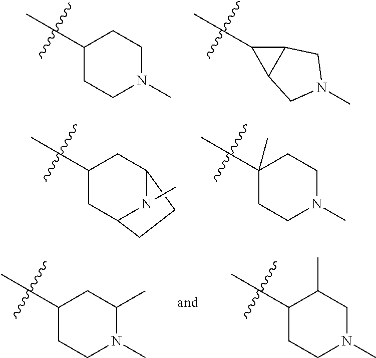

A specific value for R³ is H, halo, cyano, —C(═O)NR^{a}R^{b}, (C₁-C₆)alkoxycarbonyl, (C₂-C₆)alkenyl, or (C₁-C₆)alkyl that is optionally substituted with one or more substituents independently selected from the group consisting of hydroxy, NR^{a}R^{b}, and —S(═O)₂—R^{e}.

A specific value for R³ is H, bromo, aminocarbonyl, ethoxycarbonyl, vinyl, cyano, 1-hydroxyethyl, hydroxymethyl, N,N-dimethylaminomethyl, N-methylaminomethyl, ethyl, or methylsulfonylmethyl.

A specific compound or salt is a compound of formula (Ia):

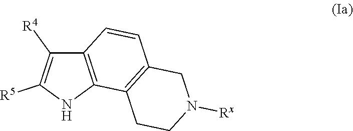

or a salt thereof.

A specific compound or salt is a compound of formula (Ic):

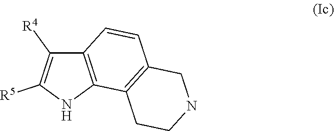

wherein the piperidine ring is optionally substituted with 1, 2, 3, 4, or 5 substituents R^{x} independently selected from the group consisting of (C₁-C₆)alkyl, (C₂-C₆)alkenyl, (C₂-C₆)alkynyl, aryl, aryl(C₁-C₆)alkyl, (C₃-C₆)cycloalkyl, (C₁-C₆)alkoxycarbonyl, (C₁-C₆)alkanoyloxy, and (C₃-C₆)cycloalkyl(C₁-C₆)alkyl, wherein each R^{x} is optionally substituted with one or more groups independently selected from halo, cyano, nitro, (C₁-C₆)alkyl, and (C₁-C₆)alkoxy; or a salt thereof.

A specific value for R⁴ is a tetrazolo[1,5-a]pyridine ring.

A specific value for R⁴ is a pyridine ring that is optionally substituted with halo, cyano, NR^{c}R^{d}, —C(═O)NR^{c}R^{d}, or (C₁-C₆)alkyl that is optionally substituted with hydroxyl.

A specific value for R⁴ is a pyridine-4-yl, 3-bromopyridine-4-yl, 2-bromopyridine-4-yl, 3-methylpyridine-4-yl, 2-methylpyridine-4-yl, 2-(aminocarbonyl)pyridine-4-yl, 3-(hydroxymethyl)pyridine-4-yl, 2-aminopyridine-4-yl, 3-cyanopyridine-4-yl, 2-cyanopyridine-4-yl, 2-(1-hydroxyethyl)pyridine-4-yl, or 3-(aminocarbonyl)pyridine-4-yl.

A specific value for R⁵ is phenyl or thiophene, which phenyl or thiophene is optionally substituted with one or more substituents independently selected from the group consisting of halo, (C₁-C₆)alkoxy, and (C₁-C₆)alkyl that is optionally substituted with one or more substituents independently selected from the group consisting of halo.

A specific value for R⁵ is 4-fluorophenyl, 4-trifluoromethylphenyl, 4-methoxyphenyl, or 3-thiophene.

A specific compound is a compound of formula (Ib):

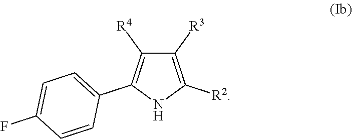

In one embodiment, each R^{a} and R^{b} is independently selected from the group consisting of H and (C₁-C₆)alkyl; or R^{a} and R^{b} together with the nitrogen to which they are attached form a morpholino, piperazino, pyrrolidino or piperidino.

In one embodiment, each R^{c} and R^{d} is independently selected from the group consisting of H and (C₁-C₆)alkyl; or R^{a} and R^{b} together with the nitrogen to which they are attached form a morpholino, piperazino, pyrrolidino or piperidino.

In one embodiment, each R^{f} and R^{g} is independently selected from the group consisting of H and (C₁-C₆)alkyl; or R^{a} and R^{b} together with the nitrogen to which they are attached form a morpholino, piperazino, pyrrolidino or piperidino.

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations: such as multiple inhalations from an insufflator or by application of a plurality of drops into the eye.

Compounds of the invention can also be administered in combination with other therapeutic agents, for example, other agents that are useful for the treatment or prevention of malaria. Examples of such agents include artimisinin. Accordingly, in one embodiment the invention also provides a composition comprising a compound of formula I, or a pharmaceutically acceptable salt thereof, at least one other therapeutic agent, and a pharmaceutically acceptable diluent or carrier. The invention also provides a kit comprising a compound of formula I, or a pharmaceutically acceptable salt thereof, at least one other therapeutic agent, packaging material, and instructions for administering the compound of formula I or the pharmaceutically acceptable salt thereof and the other therapeutic agent or agents to a human to treat or prevent malaria.

The invention will now be illustrated by the following non-limiting Examples.

### EXAMPLES

### Comparative Example 1 Synthesis of TSP

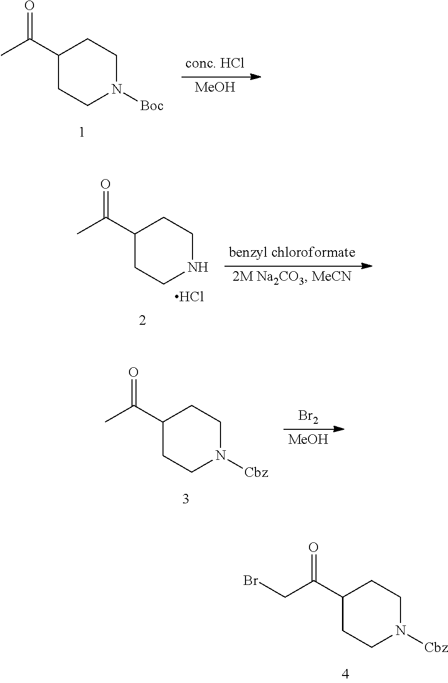

### a. 1-(piperidin-4-yl)ethan-1-one hydrochloride (2)

tert-Butyl 4-acetylpiperidine-1-carboxylate (1) (7.32 g, 32.204 mmol, 1 eq) was dissolved in 30 mL MeOH. Concentrated HCl (5 mL) was added to this and the reaction was stirred at room temperature. After 1.5 hours, the reaction was determined complete by TLC and concentrated to 5 mL under reduced pressure. A white solid precipitated out after addition of 50 mL acetone. The solution was filtered and dried to give 1-(piperidin-4-yl)ethan-1-one hydrochloride (2) (4.46 g, 85% yield) as a crystalline white solid.

### b. Benzyl 4-acetylpiperidine-1-carboxylate (3)

To a solution of 1-(piperidin-4-yl)ethan-1-one hydrochloride (2) (4.46 g, 27.349 mmol, 1 eq) in 70 mL MeCN was added 55 mL 2M Na₂CO₃. The reaction was stirred at room temperature for 15 minutes. Benzyl chloroformate (3.89 mL, density=1.2 g/mL, 27.364 mmol, 1 eq) was added dropwise and the reaction was allowed to stir overnight at room temperature. The reaction was concentrated under reduced pressure and the residue was partitioned between EtOAc and water. The aqueous phase was extracted with EtOAc (3×) and the combined organic phase was washed with brine (1×), dried over sodium sulphate and concentrated to give benzyl 4-acetylpiperidine-1-carboxylate (3) (7.09 g, 99% yield, 73% purity by LC/MS) as a white solid. Taken onto the next step without purification. (M+H)+ 261.80.

### c. Benzyl 4-(2-bromoacetyl)piperidine-1-carboxylate (4)

Benzyl 4-acetylpiperidine-1-carboxylate (3) (7.09 g, 27.13 mmol, 1 eq) was dissolved in 80 mL MeOH. Bromine (1.39 mL, density=3.1 g/mL, 27.13 mmol, 1 eq) was separately dissolved in 10 mL MeOH. The methanolic solution of bromine was added to the methanolic solution of (3) portion wise over a period of 15 minutes and the reaction was allowed to stir at room temperature. After 2.5 hours, the reaction was determined complete by TLC and LC/MS analysis and the reaction was concentrated under reduced pressure. The residue was partitioned between EtOAc and water. The aqueous phase was extracted with EtOAc (3×) and the combined organic phase was washed with brine (1×), dried over sodium sulphate and concentrated. Purification by silica gel chromatography (10%→15%→20%→25% EtOAc/Hexanes) afforded benzyl 4-(2-bromoacetyl)piperidine-1-carboxylate (4) (3.3 g, 37% yield, 93% purity by LC/MS) as a clear yellow viscous liquid. (M+H)+ 339.60, 341.60.

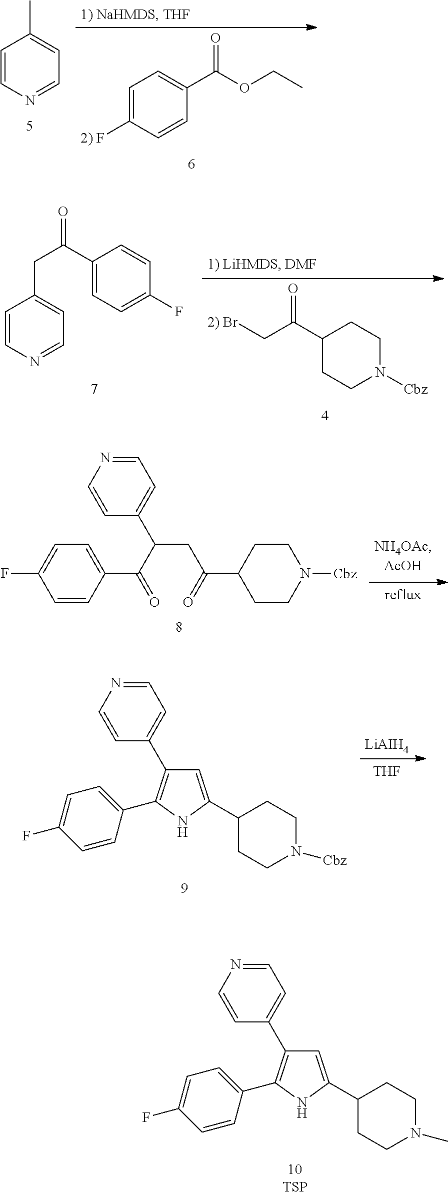

### d. 1-(4-fluorophenyl)-2-(pyridin-4-yl)ethan-1-one (7)

4-methylpyridine (5) (3.98 g, 42.74 mmol, 1 eq) was dissolved in 10 mL and cooled to 0° C. under nitrogen. NaHMDS 1M solution in THF (64.1 mL, 64.1 mmol, 1.5 eq) was added to the THF solution of 5 by means of a syringe in a portion wise manner over 10 minutes. The reaction was stirred for 1 hour maintaining the temperature at 0° C. Ethyl 4-fluorobenzoate (6) (5.04 g, 44.01 mmol, 1.1 eq) was dissolved in 8 mL THF and added to the anion generated by means of a syringe and the reaction was allowed to warm to room temperature. After 3.5 hours the reaction was determined complete by LC/MS analysis, quenched by addition of 3 mL water and concentrated under reduced pressure. The residue was partitioned between EtOAc and water. The aqueous phase was extracted with EtOAc (3×) and the combined organic phase was washed with brine, dried over sodium sulphate and concentrated. Purification by silica gel chromatography (25%→37%→50%→75% EtOAc/Hexanes) afforded 1-(4-fluorophenyl)-2-(pyridin-4-yl)ethan-1-one (7) (4.91 g, 53% yield, 98% purity by LC/MS) as a yellow solid. (M+H)+ 215.55.

### e. Benzyl 4-(4-(4-fluorophenyl)-4-oxo-3-(pyridin-4-yl)butanoyl)piperidine-1-carboxylate (8)

To a solution of 1-(4-fluorophenyl)-2-(pyridin-4-yl)ethan-1-one (7) (1.86 g, 8.64 mmol, 1 eq) in 20 mL anhydrous DMF was added LiHMDS 1M solution in THF (10.4 mL, 10.4 mmol, 1.2 eq) under nitrogen. The reaction was stirred at room temperature for 1 hour. Benzyl 4-(2-bromoacetyl)piperidine-1-carboxylate (4) (4.18 g, 12.29 mmol, 1.4 eq) was dissolved in 12 mL anhydrous DMF and added to the anion generated by means of a syringe. The reaction was stirred at room temperature and was determined complete in 45 minutes by LC/MS analysis. The reaction was concentrated under reduced pressure to remove most of the DMF. The concentrate was partitioned between water and EtOAc and the aqueous phase was extracted with EtOAc (3×). The combined organic phase was washed with brine (1×), dried over sodium sulphate and concentrated. Excess DMF was chased by forming an azeotropic mixture with toluene to give benzyl 4-(4-(4-fluorophenyl)-4-oxo-3-(pyridin-4-yl)butanoyl)piperidine-1-carboxylate (8) (6.0 g, 34% purity by LC/MS) as a dark maroon viscous liquid which was taken onto the next step without any purification. (M+H)+ 474.75.

### f. Benzyl 4-(5-(4-fluorophenyl)-4-(pyridin-4-yl)-1H-pyrrol-2-yl)piperidine-1-carboxylate (9)

Crude benzyl 4-(4-(4-fluorophenyl)-4-oxo-3-(pyridin-4-yl)butanoyl)piperidine-1-carboxylate (8) (6.0 g) was dissolved in 30 mL glacial AcOH. Ammonium acetate (5 g) was added in excess and the reaction was heated to reflux. After 1.5 hours, the reaction was deemed complete by LC/MS analysis and concentrated under reduced pressure to drive away most of the AcOH. The concentrate was partitioned between water and EtOAc and the aqueous phase was extracted with EtOAc (3×). The combined organic was washed with brine (1×), dried over sodium sulphate and concentrated. Excess AcOH was chased by forming an azeotropic mixture with toluene. Purification by silica gel chromatography (25%→37%→50% EtOAc/Hexanes) afforded benzyl 4-(5-(4-fluorophenyl)-4-(pyridin-4-yl)-1H-pyrrol-2-yl)piperidine-1-carboxylate (9) (2.64 g, 84% purity by LC/MS) as a yellow solid. (M+H)+ 455.90.

### g. 4-(2-(4-fluorophenyl)-5-(1-methylpiperidin-4-yl)-1H-pyrrol-3-yl)pyridine (10) [TSP]

To a solution of benzyl 4-(5-(4-fluorophenyl)-4-(pyridin-4-yl)-1H-pyrrol-2-yl)piperidine-1-carboxylate (9) (60 mgs, 0.132 mmol, 1 eq) in 5 mL anhydrous THF, Lithium aluminium hydride 2M solution in THF (0.26 mL, 0.526 mmol, 4 eq) was added by means of a syringe. The reaction was stirred at room temperature and was determined complete in 20 minutes by LC/MS analysis. The reaction was quenched using the Feiser & Feiser quench (0.02 mL water, 0.02 mL 15% NaOH and 0.06 mL water in a sequential manner) and stirred for an additional 30 minutes. The reaction was filtered over celite and the filtrate was concentrated. Purification by silica gel chromatography (2%→4%→6%→8% MeOH/CHCl₃ with 0.25% NH₃) afforded 4-(2-(4-fluorophenyl)-5-(1-methylpiperidin-4-yl)-1H-pyrrol-3-yl)pyridine (10) [TSP] (30 mgs, 68% yield, 100% purity by LC/MS) as a clear white solid. ¹H NMR (400 MHz, DMSO-d₆) δ 11.08 (d, J=2.4 Hz, 1H), 8.38-8.25 (m, 2H), 7.37-7.29 (m, 2H), 7.24-7.15 (m, 2H), 7.15-7.11 (m, 2H), 6.11 (dd, J=2.7, 0.8 Hz, 1H), 2.81 (dt, J=12.0, 3.2 Hz, 2H), 2.47-2.41 (m, 1H), 2.16 (s, 3H), 1.98-1.84 (m, 4H), 1.63 (qd, J=12.4, 3.7 Hz, 2H). (M+H)+ 336.30.

### Example 1. Synthesis of 3-bromo-4-(2-(4-fluorophenyl)-5-(1-methylpiperidin-4-yl)-1H-pyrrol-3-yl)pyridine

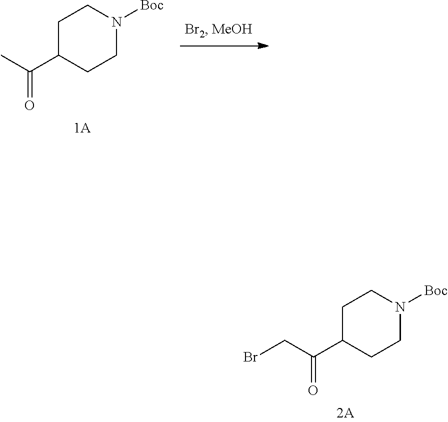 [SMILES: CC(=O)C1CCN(C(=O)OC(C)(C)C)CC1 ; CC(C)(C)OC(=O)N1CCC(C(=O)CBr)CC1]

### a. tert-Butyl 4-(2-bromoacetyl)piperidine-1-carboxylate (2A)

To a solution of tert-butyl 4-acetylpiperidine-1-carboxylate (1A) (25 g, 110 mmol, 1 eq) in MeOH (300 mL) was added bromine (5.6 mL, 110 mmol, 1.0 eq) dropwise. The reaction was monitored by TLC (25% EtOAc/Hex) staining with PMA and when complete was concentrated under reduced pressure. The residue was partitioned in EtOAc/water, the aqueous was separated and the organic was washed 1× brine, dried over Na₂SO₄, filtered and concentrated. The crude product was purified by silica gel chromatography (25% EtOAc/Hex) to give (2A) (11.5 g, 37.5 mmol) as a clear oil that crystallized on standing.

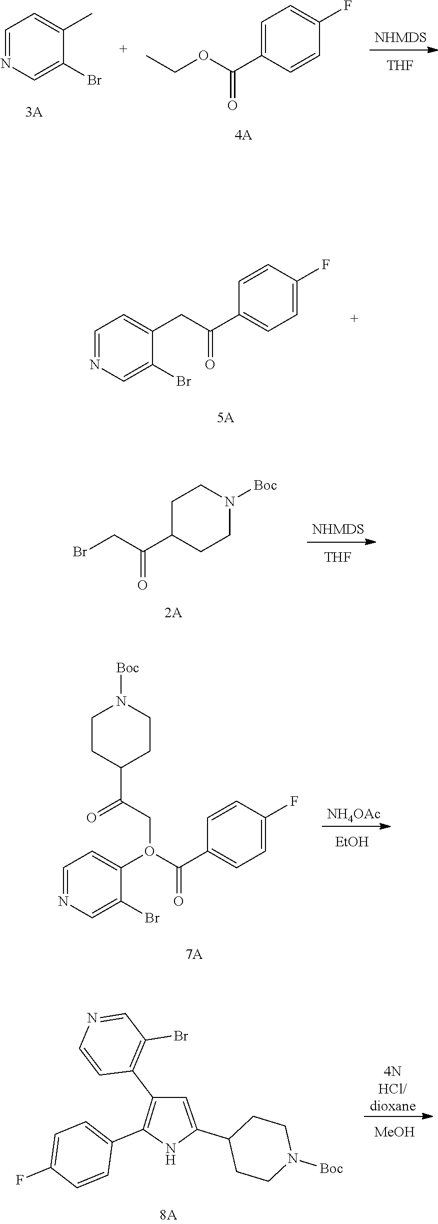

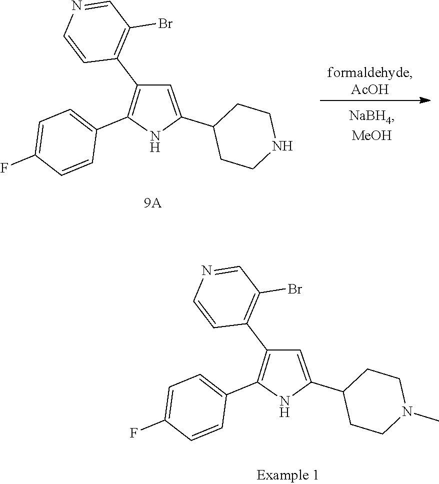

### b. 2(3-Bromopyridin-4-yl)-1-(4-fluorophenyl)ethan-1-one (5A)

3-Bromo-4-methylpyridine (3A) (3.0 g, 17.4 mmol, 1.0 eq) in THF (6 mL) was added rapidly to a solution of NHMDS (1M in THF, 26.1 mL, 26.1 mmol, 1.5 eq) at 0° C. The reaction was stirred for 30 min at this temperature followed by addition of ethyl 4-fluorobenzoate (4A) (2.93 g, 17.4 mmol, 1.0 eq) in THF (6 mL). The reaction was stirred for 1 hour at 0° C. and was allowed to slowly warm to room temperature with overnight stirring. The reaction was quenchedle with water and partitioned in Et₂O and dilute aqueous HCl. The aqueous was extracted 2×Et₂O, and the combined organic was washed with brine, dried over Na₂SO₄, filtered and concentrated. The residue was purified by silica gel chromatography (10%→25%→50% EtOAc/Hex) to give (5A) (2.95 g, 10.0 mmol, 99% purity by HPLC) as a yellow oil that crystallized on standing. MS: 293.70, 295.65 [M+H]⁺.

### c. tert-Butyl 4-(3-(3-bromopyridin-4-yl)-4-(4-fluorophenyl)-4-oxobutanoyl)piperidine-1-carboxylate (7A)

2-(3-Bromopyridin-4-yl)-1-(4-fluorophenyl)ethan-1-one (5A) (2.94 g, 10 mmol, 1.0 eq) was dissolved in THF (100 mL) and cooled to 0° C. To this solution was added NHMDS (1M in THF, 11.0 mL, 11.0 mmol, 1.1 eq). After stirring for 10 min at 0° C., a solution of tert-butyl 4-(2-bromoacetyl)piperidine-1-carboxylate (2A) (3.37 g, 11.0 mmol, 1.1 eq) in THF (10 mL) was added and the reaction was stirred for 30 min at constant temperature before warming to room temperature. The reaction seemed to stall at ˜50% completion as monitored by LC/MS. Additional quantities of base and alpha-bromoketone were added with no further reaction progression. After quenching with water, the reaction was partitioned in EtOAc/water. The organic was washed 1× water, 1× brine, dried over Na₂SO₄, filtered and concentrated to afford crude (7A) which was used as is without further purification. MS: 518.80, 520.75 [M+Na]⁺.

### d. tert-Butyl 4-(4-(3-bromopyridin-4-yl)-5-(4-fluorophenyl)-1H-pyrrol-2-yl)piperidine-1-carboxylate (8A)

Crude tert-butyl 4-(3-(3-bromopyridin-4-yl)-4-(4-fluorophenyl)-4-oxobutanoyl)piperidine-1-carboxylate (7A) was dissolved in EtOH (20 mL). NH₄OAc (3 g) was added and the reaction was heated overnight at reflux. The crude reaction was concentrated under reduced pressure and partitioned in EtOAc/water. The organic was washed 2× water, 1× brine, dried over Na₂SO₄, filtered and concentrated. The residue was purified by silica gel chromatography (10%→25%→50% EtOAc/Hex). Product fractions were combined and concentrated to afford (8A) (1.82 g, 80% purity) as a light orange solid that was used in the next reaction without further purification. MS: 499.75, 502.05 [M+H]⁺.

### e. 3-Bromo-4-(2-(4-fluorophenyl)-5-(piperidin-4-yl)-1H-pyrrol-3-yl)pyridine (9A)

tert-Butyl 4-(4-(3-bromopyridin-4-yl)-5-(4-fluorophenyl)-1H-pyrrol-2-yl)piperidine-1-carboxylate (8A) (1.82 g, 3.64 mmol, 80% purity) was dissolved in MeOH (15 mL). To this solution was added HCl (4N in dioxane, 4 mL). After stirring for 2 hours at room temperature, the reaction was determined complete by LC/MS and concentrated under reduced pressure. The residue was partitioned in EtOAc/water, making sure that the pH<4. The aqueous was separated, washed 2×DCM and basified with NaHCO₃ (sat, aq). The basic aqueous was extracted 4× (5% MeOH/DCM), and the combined organic was dried over Na₂SO₄, filtered and concentrated to give (9A) (1.19 g, 2.97 mmol, 99% purity by LC/MS, 30% yield over 3 steps) as an orange solid. MS: 399.55, 401.6 [M+H]⁺.

### f. 3-Bromo-4-(2-(4-fluorophenyl)-5-(1-methylpiperidin-4-yl)-1H-pyrrol-3-yl)pyridine

3-Bromo-4-(2-(4-fluorophenyl)-5-(piperidin-4-yl)-1H-pyrrol-3-yl)pyridine (9A), (500 mg, 1.25 mmol, 1 eq) was dissolved in MeOH (10 mL), followed by addition of formaldehyde (37% aq, 2 mL) and AcOH (0.5 mL). The reaction was stirred for 2 hours at room temperature upon which an aliquot was quenched with excess NaBH₄. The reaction continued stirring at room temperature until aliquot quenched with NaBH₄, showed complete conversion to methylated product by LC/MS (this indicates that that imine formation is complete). At this time, the remainder of the reaction was quenched portionwise with NaBH₄, and monitored by LC/MS after each addition until complete conversion to methylated product (no starting material 9A remaining). The crude reaction was partitioned in EtOAc/1N NaOH (aq). The organic was separated and extracted 2× dilute aqueous HCL. The combined acidic aqueous was basified with 1 N NaOH to pH=9 and extracted 3×DCM. The combined organic was dried over Na₂SO₄, filtered and concentrated to afford the title compound Example 1 (295 mg, 0.737 mmol, 99% purity by HPLC) as a light yellow solid. MS: 413.65, 415.65 [M+H]⁺. 1H NMR (400 MHz, DMSO-Δ6) δ 11.14 (s, 1H), 8.68 (s, 1H), 8.36 (d, J=4.9 Hz, 1H), 7.20-7.06 (m, 5H), 5.96 (dd, J=2.6, 0.8 Hz, 1H), 2.87-2.79 (m, 2H), 2.53 (dt, J=7.6, 3.9 Hz, 1H), 2.17 (s, 3H), 1.99-1.87 (m, 4H), 1.63 (qd, J=12.5, 3.8 Hz, 2H).

### Example 2. Synthesis of 2-bromo-4-(2-(4-fluorophenyl)-5-(1-methylpiperidin-4-yl)-1H-pyrrol-3-yl)pyridine

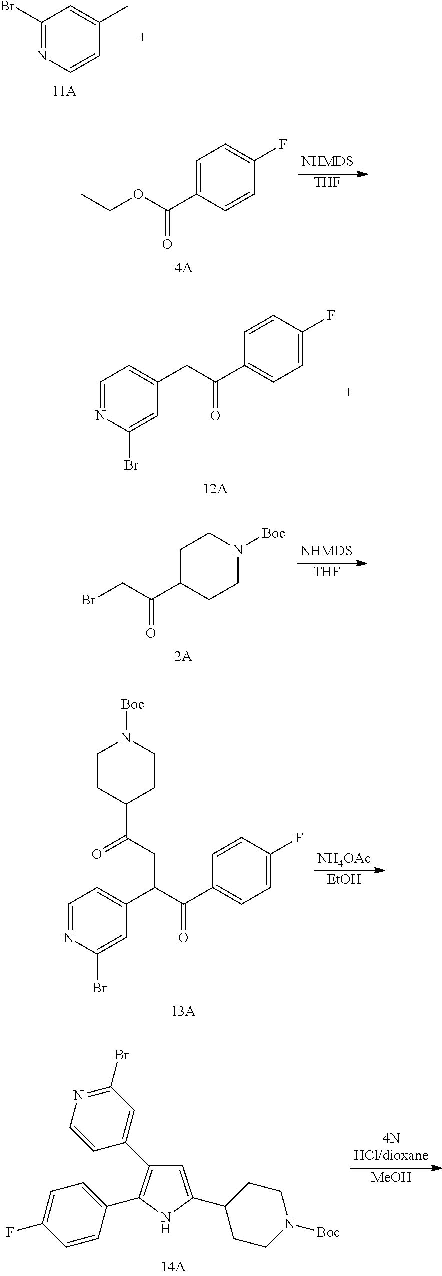

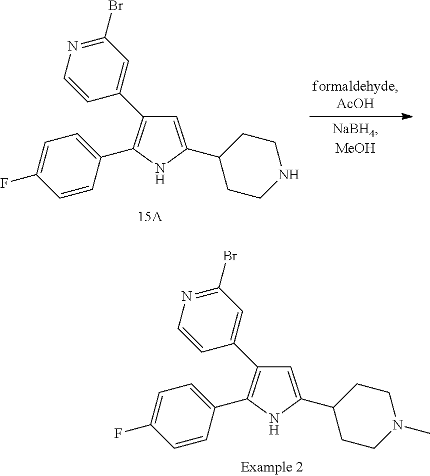 [SMILES: Fc1ccc(-c2[nH]c(C3CCNCC3)cc2-c2ccnc(Br)c2)cc1 ; CN1CCC(c2cc(-c3ccnc(Br)c3)c(-c3ccc(F)cc3)[nH]2)CC1]

### a. 2-(2-Bromopyridin-4-yl)-1-(4-fluorophenyl)ethan-1-one (12A)

Following the procedure as described for the synthesis of (5A) using 2-bromo-4-methylpyridine (11A) (3.0 g, 17.4 mmol, 1 eq) and ethyl 4-fluorobenzoate (4A) (2.93 g, 17.4 mmol, 1 eq), the compound (12A) (2.0 g, 6.84 mmol, 39% yield, 99% purity by HPLC) was isolated as a white solid. MS: 293.65, 295.6 [M+H]⁺.

### b. tert-Butyl 4-(3-(2-bromopyridin-4-yl)-4-(4-fluorophenyl)-4-oxobutanoyl)piperidine-1-carboxylate (13A)

Following the procedure as described for the synthesis of (7A) using 2-(2-bromopyridin-4-yl)-1-(4-fluorophenyl)ethan-1-one (12A) (1.21 g, 4.1 mmol, 1.0 eq) and tert-butyl 4-(2-bromoacetyl)piperidine-1-carboxylate (2A) (1.38 g, 4.51 mmol, 1.1 eq), the compound (13A) was carried on crude after workup to the next reaction. MS: 540.65, 542.75 [M+Na]⁺.

### c. tert-Butyl 4-(4-(2-bromopyridin-4-yl)-5-(4-fluorophenyl)-1H-pyrrol-2-yl)piperidine-1-carboxylate (14A)

Following the procedure as described for the synthesis of (8A) using crude tert-butyl 4-(3-(2-bromopyridin-4-yl)-4-(4-fluorophenyl)-4-oxobutanoyl)piperidine-1-carboxylate (13A) (2.13 g, 4.1 mmol), the title compound (14A) (1.63 g, 3.26 mmol, 89% yield over 2 steps, 99% purity by HPLC) was isolated as a yellow solid. MS: 500.80, 501.85 [M+H]⁺.

### d. 2-Bromo-4-(2-(4-fluorophenyl)-5-(piperidin-4-yl)-1H-pyrrol-3-yl)pyridine (15A)

Following the procedure as described for the synthesis of (9A) using tert-butyl 4-(4-(2-bromopyridin-4-yl)-5-(4-fluorophenyl)-1H-pyrrol-2-yl)piperidine-1-carboxylate (14A) (657 mg, 1.31 mmol), the compound (15A) (427 mg, 1.07 mmol, 82% yield, 99% purity by HPLC) was isolated as a light yellow solid. MS: 399.70, 401.70 [M+H]⁺.

### e. 2-Bromo-4-(2-(4-fluorophenyl)-5-(1-methylpiperidin-4-yl)-1H-pyrrol-3-yl)pyridine

Following the procedure as described for the synthesis of Example 1, but using 2-bromo-4-(2-(4-fluorophenyl)-5-(piperidin-4-yl)-1H-pyrrol-3-yl)pyridine (15A) (425 mg, 1.06 mmol), the title compound Example 2 (414 mg, 1.0 mmol, 94% yield, 99%/o purity by HPLC) was isolated as a light orange solid. MS: 413.65, 415.55 [M+H]⁺. ¹H NMR (400 MHz, DMSO-66) δ 11.20 (s, 1H), 8.10 (dd, J=5.3, 0.6 Hz, 1H), 7.43-7.29 (m, 3H), 7.28-7.18 (m, 2H), 7.13 (dd, J=5.3, 1.6 Hz, 1H), 6.20 (dd, J=2.5, 0.8 Hz, 1H), 3.27 (m, 1H), 2.81 (dt, J=12.1, 3.3 Hz, 2H), 2.16 (s, 3H), 1.91 m, 4H), 1.62 (qd, J=12.2, 3.8 Hz, 2H).

### Example 3. Synthesis of 4-(2-(4-fluorophenyl)-5-(1-methylpiperidin-4-yl)-1H-pyrrol-3-yl)-3-methylpyridine

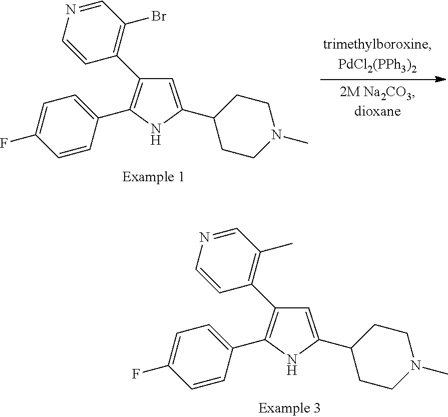 [SMILES: CN1CCC(c2cc(-c3ccncc3Br)c(-c3ccc(F)cc3)[nH]2)CC1 ; Cc1cnccc1-c1cc(C2CCN(C)CC2)[nH]c1-c1ccc(F)cc1]

### a. 4-(2-(4-Fluorophenyl)-5-(1-methylpiperidin-4-yl)-1H-pyrrol-3-yl)-3-methylpyridine

3-Bromo-4-(2-(4-fluorophenyl)-5-(1-methylpiperidin-4-yl)-1H-pyrrol-3-yl)pyridine (Example 1, 50 mg, 0.12 mmol, 1.0 eq), trimethylboroxine (50% wt. in THF, 60 mg, 0.24 mmol, 2.0 eq), PdCl₂(PPh₃)₂ (4 mg, 0.006 mmol, 0.05 eq), dioxane (1 mL) and Na₂CO₃ (2M in water, 0.5 mL) were combined and heated in a microwave reactor for 10 minutes at 130° C. The reaction was determined complete by LC/MS, partitioned in EtOAc/water and filtered over celite to remove insoluble impurities. The organic layer was separated and washed 2× water. The organic was then extracted 2×1N HCl. The combined acidic aqueous was basified with aqueous NaOH and extracted 3×DCM. The combined DCM extracts were dried over Na₂SO₄, filtered and concentrated. The concentrate was purified by silica gel chromatography (0%→5%→10% MeOH/DCM with 1% 7N NH₃ in MeOH as a modifier) to yield the title compound Example 3 (32.5 mg, 0.093 mmol, 94% purity by HPLC) as a yellow solid after concentration and drying under vacuum. MS: 349.95 [M+H]⁺. ¹H NMR (400 MHz, DMSO-86) δ 11.03 (s, 1H), 8.34 (s, 1H), 8.23 (d, J=5.0 Hz, 1H), 7.17-7.04 (m, 4H), 7.01 (d, J=5.0 Hz, 1H), 5.86 (d, J=2.6 Hz, 1H), 2.86-2.77 (m, 2H), 2.56-2.49 (m, 1H), 2.16 (s, 3H), 1.97 (s, 3H), 1.96-1.86 (m, 4H), 1.63 (qd, J=12.4, 3.7 Hz, 2H).

### Example 4. Synthesis of 4-(2-(4-fluorophenyl)-5-(1-methylpiperidin-4-yl)-1H-pyrrol-3-yl)-2-methylpyridine

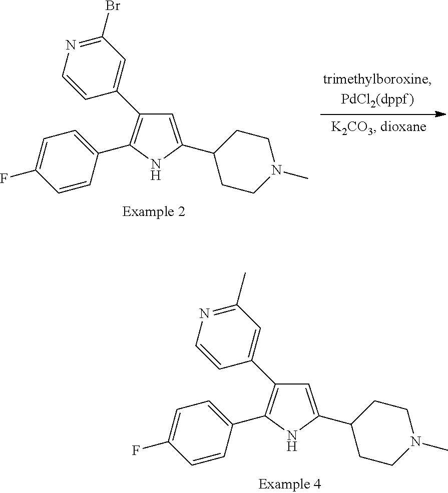 [SMILES: CN1CCC(c2cc(-c3ccnc(Br)c3)c(-c3ccc(F)cc3)[nH]2)CC1 ; Cc1cc(-c2cc(C3CCN(C)CC3)[nH]c2-c2ccc(F)cc2)ccn1]

### a. 4-(2-(4-Fluorophenyl)-5-(1-methylpiperidin-4-yl)-1H-pyrrol-3-yl)-2-methylpyridine

2-Bromo-4-(2-(4-fluorophenyl)-5-(1-methylpiperidin-4-yl)-1H-pyrrol-3-yl)pyridine (Example 2, 50 mg, 0.121 mmol, 1 eq), PdCl₂ (dppf) (4.4 mg, 0.00605 mmol, 0.05 eq) and K₂CO₃ (67 mg, 0.484 mmol, 4.0 eq) were taken up in dioxane (2 mL), followed by addition of trimethylboroxine (50% wt. in THF, 46 mg, 0.182 mmol, 1.5 eq). The reaction was degassed and stirred overnight at reflux under N₂. Subsequent addition of catalyst (4 mg) and boroxine (46 mg) and heating overnight (˜48 hours total) was needed to push the reaction to completion. Workup and purification were performed as described for Example 3, to afford the title compound Example 4 (5.2 mg, 0.0149 mmol, 98% purity by HPLC) as a yellow solid. MS: 349.95 [M+H]⁺. ¹H NMR (400 MHz, DMSO-66) δ 11.04 (s, 1H), 8.17 (d, J=5.2 Hz, 1H), 7.36-7.28 (m, 2H), 7.22-7.14 (m, 2H), 7.08-7.04 (m, 1H), 6.90-6.85 (m, 1H), 6.08 (d, J=2.5 Hz, 1H), 2.82 (d, J=11.2 Hz, 2H), 2.33 (s, 3H), 2.17 (s, 3H), 1.99-1.84 (m, 4H), 1.62 (qd, J=12.4, 3.7 Hz, 2H).

### Example 5 Synthesis of 4-(2-(4-fluorophenyl)-5-(1-methylpiperidin-4-yl)-1H-pyrrol-3-yl)picolinamide

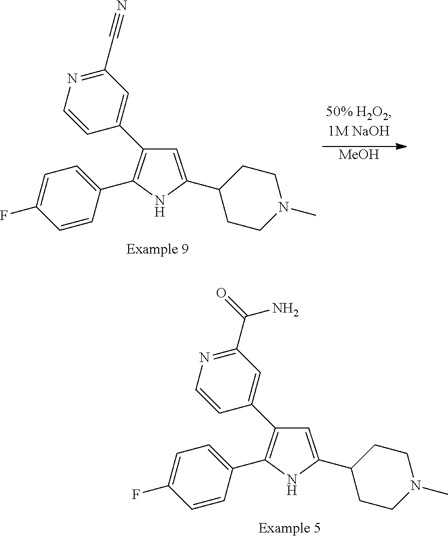 [SMILES: "MeOH" ; CN1CCC(c2cc(-c3ccnc(C#N)c3)c(-c3ccc(F)cc3)[nH]2)CC1 ; CN1CCC(c2cc(-c3ccnc(C(N)=O)c3)c(-c3ccc(F)cc3)[nH]2)CC1]

### a. 4-(2-(4-Fluorophenyl)-5-(1-methylpiperidin-4-yl)-1H-pyrrol-3-yl)picolinamide

To a solution of 4-(2-(4-fluorophenyl)-5-(1-methylpiperidin-4-yl)-1H-pyrrol-3-yl)picolinonitrile (Example 9, 50 mg, 0.139 mmol, 1 eq) in MeOH (5 mL) was added NaOH (1M aq., 500 μL) followed by H₂O₂ (50% aq., 100 μL). The reaction was determined complete by LC/MS after stirring for 30 minutes at room temperature and was partitioned in dilute aq. HCl (pH=3) and EtOAc. The aqueous was washed 2×EtOAc and basified with 1M NaOH. The basic aqueous was extracted 3×DCM and the combined organic was dried over Na₂SO₄, filtered and concentrated. The residue was purified by silica gel chromatography (0%→5%→10% MeOH/DCM with 1% 7N NH₃ in MeOH as a modifier) to afford the title compound Example 5 (28.1 mg, 0.074 mmol, >99% purity by HPLC) as a yellow solid. MS: 378.80 [M+H]⁺. 1H NMR (400 MHz, DMSO-56) δ 11.16 (d, J=2.7 Hz, 1H), 8.36 (dd, J=5.1, 0.7 Hz, 1H), 7.99 (d, J=3.0 Hz, 1H), 7.84 (dd, J=1.9, 0.8 Hz, 1H), 7.51 (d, J=3.0 Hz, 1H), 7.37-7.31 (m, 2H), 7.29 (dd, J=5.1, 1.9 Hz, 1H), 7.24-7.15 (m, 2H), 6.16 (dd, J=2.7, 0.8 Hz, 1H), 2.82 (dt, J=11.0, 2.6 Hz, 3H), 2.17 (s, 3H), 1.98-1.87 (m, 4H), 1.64 (qd, J=12.3, 3.6 Hz, 2H).

### Example 6. Synthesis of (4-(2-(4-fluorophenyl)-5-(1-methylpiperidin-4-yl)-1H-pyrrol-3-yl)pyridin-3-yl)methanol

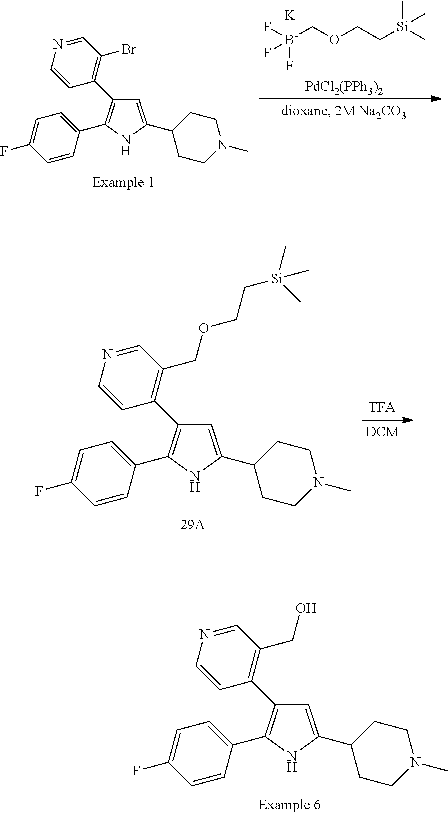 [SMILES: CN1CCC(c2cc(-c3ccncc3Br)c(-c3ccc(F)cc3)[nH]2)CC1 ; C[Si](C)(C)CCOC[B-](F)(F)F ; [K+] ; CN1CCC(c2cc(-c3ccncc3COCC[Si](C)(C)C)c(-c3ccc(F)cc3)[nH]2)CC1 ; CN1CCC(c2cc(-c3ccncc3CO)c(-c3ccc(F)cc3)[nH]2)CC1 ; "PdCl2(PPh3)2"]

### a. 4-(4-Fluorophenyl)-5-(1-methylpiperidin-4-yl)-1H-pyrrol-3-yl)-3-((2-(trimethylsilyl)ethoxy) methyl)pyridine (29A)

A mixture of Example 1 (60 mg, 0.145 mmol, 1.0 eq), Potassium [(2-trimethylsilyl)ethoxymethyl]trifluoroborate (104 mg, 0.435 mmol, 3.0 eq), PdCl₂(PPh₃)₂ (4 mg, 0.00605 mmol, 0.05 eq), dioxane (1 mL) and Na₂CO₃ (2M aq., 0.5 mL) were heated in a microwave reactor for 10 minutes at 130° C. The reaction was determined complete by LC/MS and partitioned in EtOAc/water. The aqueous was separated and further extracted 1×EtOAc. The organic was combined, dried over Na₂SO₄, filtered and concentrated. The residue was purified by silica gel chromatography (0%→5%→10% MeOH/DCM with 1% 7N NH₃ in MeOH as a modifier) to afford (29A) (31.2 mg, 0.0670 mmol, 100% purity by HPLC) as a yellow solid after concentration. MS: 465.80 [M+H]⁺.

### b. (4-(2-(4-Fluorophenyl)-5-(1-methylpiperidin-4-yl)-1H-pyrrol-3-yl)pyridin-3-yl)methanol

To a solution of (29A) (30.6 mg, 0.0657 mmol, 1.0 eq) in DCM (1 mL) was added TFA (1 mL). The reaction was determined complete by LC/MS after 1 hour and concentrated under reduce pressure. The residue was partitioned in EtOAc/1M NaOH (aq), and the organic was separated, dried over Na₂SO₄, filtered and concentrated. The concentrate was purified by silica gel chromatography (0%→5%→10% MeOH/DCM with 1% 7N NH₃ in MeOH as a modifier) and concentrated to afford the title compound Example 6 (18.4 mg, 0.050 mmol, >99% purity by HPLC) as an orange solid. MS: 365.55 [M+H]⁺. ¹H NMR (400 MHz, DMSO-66) δ 11.08 (s, 1H), 8.59 (s, 1H), 8.27 (d, J=5.1 Hz, 1H), 7.21-7.04 (m, 5H), 6.96 (d, J=5.0 Hz, 1H), 5.93 (d, J=2.5 Hz, 1H), 5.07 (t, J=5.4 Hz, 1H), 4.28 (d, J=5.5 Hz, 2H), 2.94 (s, 2H), 2.30 (d, J=14.1 Hz, 3H), 1.96 (d, J=7.9 Hz, 2H), 1.66 (q, J=11.8 Hz, 2H).

### Example 7. Synthesis of 4-(2-(4-fluorophenyl)-5-(1-methylpiperidin-4-yl)-1H-pyrrol-3-yl)pyridin-2-amine

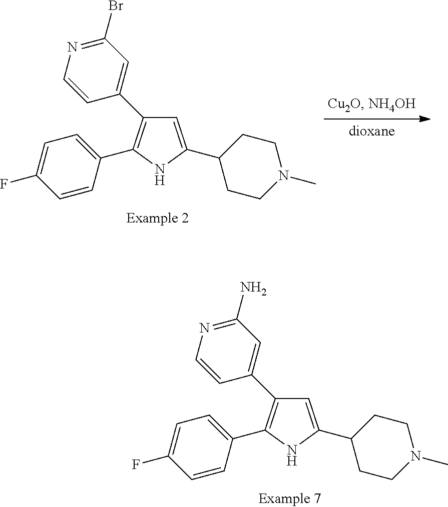 [SMILES: CN1CCC(c2cc(-c3ccnc(Br)c3)c(-c3ccc(F)cc3)[nH]2)CC1 ; CN1CCC(c2cc(-c3ccnc(N)c3)c(-c3ccc(F)cc3)[nH]2)CC1]

### a. 4-(2-(4-Fluorophenyl)-5-(1-methylpiperidin-4-yl)-1H-pyrrol-3-yl)pyridin-2-amine

A mixture of Example 2 (100 mg, 0.241 mmol, 1.0 eq) and Cu₂O (103 mg, 0.723 mmol, 3.0 eq) in dioxane (1 mL) and NH₄OH (1 mL) was heated in a microwave reactor for 30 minutes at 140° C. to give a 2:1 mixture of amine:alcohol by LC/MS. The crude mixture was partitioned in EtOAc/water and the organic was washed 2× water, and filtered over celite. The organic filtrate was extracted 2× dilute aqueous HCl. The combined acidic aqueous was basified with 1M NaOH and extracted 3×DCM. The combined organic was dried over Na₂SO₄, filtered and concentrated. The crude residue was purified by silica gel chromatography (0%→5%→10% MeOH/DCM with 1% 7N NH₃ in MeOH as a modifier) to afford the title compound Example 7 (12.0 mg, 0.034 mmol, 99% purity by HPLC) as a yellow solid after concentration. MS: 350.8 [M+H]⁺. ¹H NMR (400 MHz, DMSO-δ6) δ 10.93 (s, 1H), 7.67 (d, J=5.4 Hz, 1H), 7.36-7.29 (m, 2H), 7.19-7.11 (m, 2H), 6.31 (dd, J=1.5, 0.8 Hz, 1H), 6.24 (dd, J=5.4, 1.5 Hz, 1H), 5.93 (dd, J=2.6, 0.7 Hz, 1H), 5.64 (s, 2H), 2.86 (d, J=10.9 Hz, 2H), 2.21 (s, 3H), 2.01 (s, 2H), 1.91 (d, J=11.9 Hz, 2H), 1.62 (qd, J=12.4, 3.7 Hz, 2H).

### Example 8. Synthesis of 4-(2-(4-fluorophenyl)-5-(1-methylpiperidin-4-yl)-1H-pyrrol-3-yl)nicotinonitrile

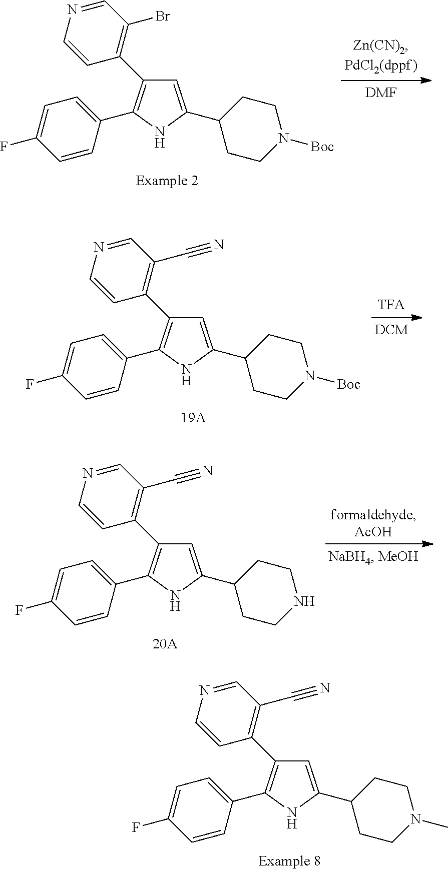

### a. tert-Butyl 4-(4-(3-cyanopyridin-4-yl)-5-(4-fluorophenyl)-1H-pyrrol-2-yl)piperidine-1-carboxylate (19A)

tert-Butyl 4-(4-(3-bromopyridin-4-yl)-5-(4-fluorophenyl)-1H-pyrrol-2-yl)piperidine-1-carboxylate (8A), (80 mg, 0.160 mmol, 1.0 eq), Zn(CN)₂ (56 mg, 0.480 mmol, 3.0 eq), PdCl₂(dppf) (59 mg, 0.080 mmol, 0.5 eq) in DMF (2 mL) were heated in a microwave reactor for 10 minutes at 170° C. and determined complete by LC/MS. The crude reaction was partitioned in EtOAc/water. The organic was separated and washed 2× water, 1× brine, dried over Na₂SO₄, filtered and concentrated. The residue was purified by silica gel chromatography (25% EtOAc/Hex) to give the compound (19A) (49.5 mg, 0.11 mmol, 99% purity by HPLC) as a light yellow solid. MS: 446.95 [M+H]⁺.

### b. 4-(2-(4-Fluorophenyl)-5-(piperidin-4-yl)-1H-pyrrol-3-yl)nicotinonitrile (20A)

To a solution of tert-butyl 4-(4-(3-cyanopyridin-4-yl)-5-(4-fluorophenyl)-1H-pyrrol-2-yl)piperidine-1-carboxylate (19A) (47 mg, 0.105 mmol, 1.0 eq) in DCM (5 mL) was added TFA (1 mL). The reaction was stirred 4 hours at room temperature, determined complete by LC/MS and concentrated under reduced pressure to afford the compound (20A) (quantitative, 100% purity by HPLC) as a yellow glassy solid. MS: 346.70 [M+H]⁺.

### c. 4-(2-(4-fluorophenyl)-5-(1-methylpiperidin-4-yl)-1H-pyrrol-3-yl)nicotinonitrile

Following the procedure as described for the synthesis of (10A) using 4-(2-(4-fluorophenyl)-5-(piperidin-4-yl)-1H-pyrrol-3-yl)nicotinonitrile (20A) (36 mg, 0.104 mmol, 1 eq), the title compound was prepared Example 8 (30.8 mg, 0.085 mmol, >990% purity by HPLC) as a light yellow solid. MS: 360.65 [M+H]⁺. ¹H NMR (400 MHz, DMSO-66) δ 11.38 (s, 1H), 8.84 (d, J=0.7 Hz, 1H), 8.56 (d, J=5.4 Hz, 1H), 7.26-7.19 (m, 3H), 7.19-7.13 (m, 2H), 6.18 (dd, J=2.6, 0.8 Hz, 1H), 2.83 (dt, J=11.8, 3.1 Hz, 2H), 2.54 (dt, J=7.4, 3.6 Hz, 1H), 2.17 (s, 3H), 2.00-1.86 (m, 4H), 1.63 (qd, J=12.3, 3.8 Hz, 2H).

### Example 9. Synthesis of 4-(2-(4-fluorophenyl)-5-(1-methylpiperidin-4-yl)-1H-pyrrol-3-yl)picolinonitrile

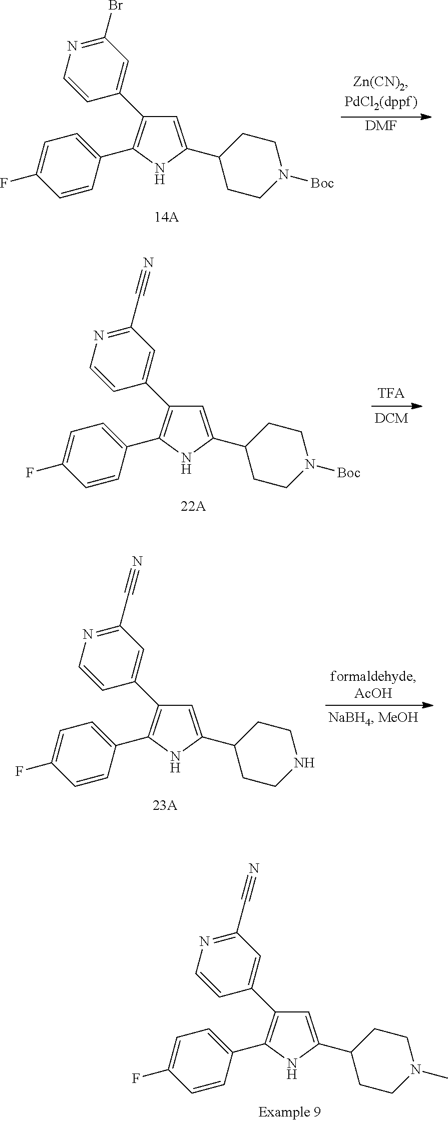

### a. tert-Butyl 4-(4-(2-cyanopyridin-4-yl)-5-(4-fluorophenyl)-1H-pyrrol-2-yl)piperidine-1-carboxylate (22A)

Following the procedure for the synthesis of (19A) using (14A) (125 mg, 0.250 mmol, 1.0 eq), the compound (22A) (86.7 mg, 0.194 mmol, >99% purity by HPLC) was isolated as a yellow solid. MS: 447.05 [M+H]⁺.

### b. 4-(2-(4-Fluorophenyl)-5-(piperidin-4-yl)-1H-pyrrol-3-yl)picolinonitrile (23A)

Following the procedure for the synthesis of (20A) using (22A) (82 mg, 0.184 mmol, 1 eq), the compound (23A) (quantitative yield, >99% purity by HPLC) was isolated as a glassy yellow solid. MS: 346.90 [M+H]⁺.

### c. 4-(2-(4-Fluorophenyl)-5-(1-methylpiperidin-4-yl)-1H-pyrrol-3-yl)picolinonitrile

Following the procedure for the synthesis of Example 8 using (23A) (64 mg, 0.184 mmol, 1 eq), the title compound Example 9 (66.1 mg, 0.183 mmol, >99% purity by HPLC) was isolated as a yellow solid. MS: 360.80 [M+H]⁺. ¹H NMR (400 MHz, DMSO-d6) δ 11.29 (s, 1H), 8.44 (dd, J=5.3, 0.7 Hz, 1H), 7.74 (dd, J=1.9, 0.8 Hz, 1H), 7.41-7.34 (m, 3H), 7.28-7.21 (m, 2H), 6.29 (d, J=2.3 Hz, 1H), 2.89 (d, J=10.7 Hz, 2H), 2.56-2.51 (m, 1H), 2.24 (s, 3H), 2.06 (s, 2H), 1.93 (d, J=12.8 Hz, 2H), 1.66 (tt, J=12.7, 6.4 Hz, 2H).

### Example 10. Synthesis of 1-(4-(2-(4-fluorophenyl)-5-(1-methylpiperidin-4-yl)-1H-pyrrol-3-yl)pyridin-2-yl)ethan-1-ol

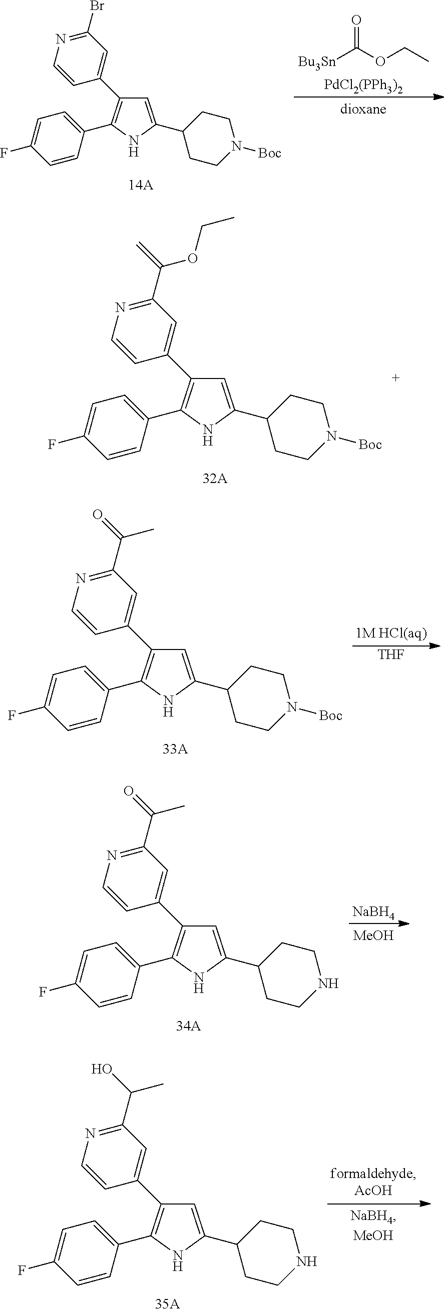

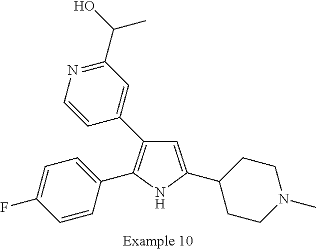 [SMILES: CC(O)c1cc(-c2cc(C3CCN(C)CC3)[nH]c2-c2ccc(F)cc2)ccn1]

### a. tert-Butyl 4-(4-(2-(1-ethoxyvinyl)pyridin-4-yl)-5-(4-fluorophenyl)-1H-pyrrol-2-yl)piperidine-1-carboxylate (32A) and tert-butyl 4-(4-(2-acetylpyridin-4-yl)-5-(4-fluorophenyl)-1H-pyrrol-2-yl)piperidine-1-carboxylate (33A)

A mixture of (14A) (98 mg, 0.196 mmol, 1.0 eq), ethyl tributylstannanecarboxylate (85 mg, 0.235 mmol, 1.2 eq), PdCl₂(PPh₃)₂ in dioxane (5 mL) was heated in a microwave reactor for 30 min at 140° C. and determined complete by LC/MS as a mixture of (32A) and (33A). The crude reaction was concentrated under reduced pressure and purified by silica gel chromatography (10%→25% EtOAc/Hex) to give a mixture of (32A) and (33A) (32.5 mg) as a yellow oil after concentration, which was used as is in the next reaction. (32A) MS: 491.90 [M+H]⁺. (33A) MS: 464.00 [M+H]⁺.

### b. 1-(4-(2-(4-Fluorophenyl)-5-(piperidin-4-yl)-1H-pyrrol-3-yl)pyridin-2-yl)ethan-1-one (34A)

To the mixture of (32A) and (33A) (32.5 mg) in THF (2 mL) was added 2 ml 1M HCl(aq). The solution was heated 1 hour at 60° C. to give complete conversion to the compound (34A) (95% purity by HPLC) which was concentrated and used as is without further purification. MS: 364.00 [M+H]⁺.

### c. 1-(4-(2-(4-fluorophenyl)-5-(piperidin-4-yl)-1H-pyrrol-3-yl)pyridin-2-yl)ethan-1-ol (35A)

To a solution of (34A) in MeOH was added an excess of NaBH₄. The reaction was stirred for 30 min at room temperature, determined complete by LC/MS and concentrated under reduced pressure. The concentrate was partitioned in EtOAc/water, the aqueous was extracted 2×EtOAc, the combined organic was dried over Na₂SO₄, filtered and concentrated to give the compound (35A) (91% purity by HPLC) which was used as is without further purification. MS: 365.80 [M+H]⁺.

### d. 1-(4-(2-(4-Fluorophenyl)-5-(1-methylpiperidin-4-yl)-1H-pyrrol-3-yl)pyridin-2-yl)ethan-1-ol

Following the procedure for the synthesis of Example 1, using (35A), the title compound Example 10 (16.5 mg, 0.0435 mmol, >99% purity by HPLC) was isolated as a yellow solid. MS: 380.15 [M+H]⁺. ¹H NMR (400 MHz, DMSO-d6) δ 11.12 (s, 1H), 8.21 (d, J=5.2 Hz, 1H), 7.40-7.29 (m, 3H), 7.25-7.13 (m, 2H), 6.96 (dd, J=5.2, 1.8 Hz, 1H), 6.11 (d, J=2.5 Hz, 1H), 5.13 (d, J=4.5 Hz, 1H), 4.65-4.55 (m, 1H), 3.11 (m, 2H), 2.43 (s, 3H), 2.03 (d, J=12.9 Hz, 2H), 1.73 (s, 2H), 1.27 (d, J=6.5 Hz, 3H).

### Example 11. Synthesis of 2-(4-fluorophenyl)-7-methyl-3-(pyridin-4-yl)-6,7,8,9-tetrahydro-1H-pyrrolo[2,3-f]isoquinoline

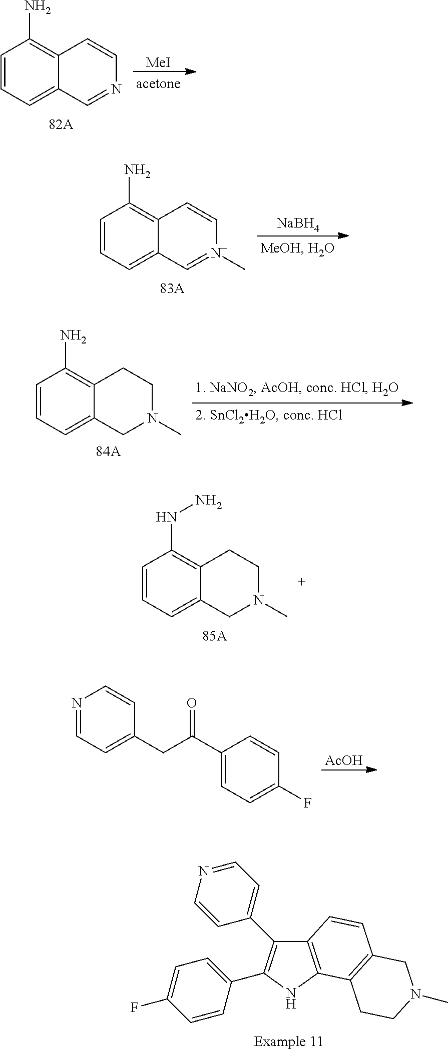

### a. 5-Amino-2-methylisoquinolin-2-ium (83A)

To a solution of isoquinolin-5-amine (82A) (2.0 g, 13.9 mmol, 1.0 eq) in acetone (42 mL) was added iodomethane (2 mL, 32.1 mmol, 2.3 eq). After stirring for 2 hours at room temperature, the precipitated yellow solid was filtered and washed with acetone. After drying under vacuum the compound (83A) (2.2 g, quantitative yield) was isolated as a yellow solid.

### b. 2-Methyl-1,2,3,4-tetrahydroisoquinolin-5-amine (84A)

To a solution of 5-amino-2-methylisoquinolin-2-ium (83A), (1.1 g, 6.9 mmol, 1.0 eq) in MeOH (132 mL) and water (11 mL) at 0° C. was added NaBH₄ (1.71 g, 45.2 mmol, 6.5 eq) portionwise. The reaction was stirred overnight after slowly warming to room temperature and was determined complete by LC/MS. The reaction was concentrated under reduced pressure to remove the majority of the methanol present. The remainder was partitioned in EtOAc/water. The organic was separated and the aqueous was extracted 2×EtOAc. The combined organic was washed 1× brine, dried over Na₂SO₄, filtered and concentrated to afford the compound (84A) (605 mg, 3.7 mmol) as a viscous brown oil that was used without further purification.

### c. 5-Hydrazineyl-2-methyl-1,2,3,4-tetrahydroisoquinoline (85A)

To a solution of 2-methyl-1,2,3,4-tetrahydroisoquinolin-5-amine (84A) (604 mg, 3.58 mmol) in AcOH (7.4 mL) and conc. HCl (7.4 mL) at 0° C. was added a solution of NaNO₂ (297 mg, 4.3 mmol, 1.2 eq) in water (4 mL). After stirring for 30 min, a solution of SnCl₂.2H₂O (2.70 g, 12.0 mmol, 3.4 eq) in conc. HCl (7.4 mL) was added. The reaction was allowed to slowly warm to room temperature and was stirred overnight. The reaction was determined complete by LC/MS and filtered to remove inorganic solids. The filtrate was concentrated to afford the title compound (85A) (assumed 20% purity) as an oil that was used without further purification.

### d. 2-(4-fluorophenyl)-7-methyl-3-(pyridin-4-yl)-6,7,8,9-tetrahydro-1H-pyrrolo[2,3-f]isoquinoline

5-Hydrazineyl-2-methyl-1,2,3,4-tetrahydroisoquinoline (85A) (247 mg, 20% wt., 0.279 mmol, 1.0 eq) and 1-(4-fluorophenyl)-2-(pyridin-4-yl)ethan-1-one (60 mg, 0.279 mmol, 1 eq) were taken up in AcOH (10 mL) and heated in a microwave reactor for 30 minutes at 130° C. The reaction was determined sufficiently complete by LC/MS to carny forward to purification. The precipitated solid was filtered and washed with minimal AcOH. The solid (60% purity by HPLC) was partitioned in 1M NaOH(aq) and DCM. The aqueous was extracted 6×DCM. The combined organics were dried over Na₂SO₄, filtered and concentrated. The residue was purified by silica gel chromatography (0%→5%→10% MeOH/DCM with 1% 7N NH₃ in MeOH as a modifier). Product fractions were combined, concentrated and recrystallized from MeOH. The solid was washed with methanol and dried under vacuum to yield the title compound Example 11 (21.7 mg, 0.0607 mmol, 99% purity by HPLC) as a yellow/brown solid. MS: 358.3 [M+H]⁺. ¹H NMR (400 MHz, DMSO-d6) δ 11.45 (s, 1H), 8.52-8.46 (m, 2H), 7.51-7.43 (m, 2H), 7.37 (d, J=8.2 Hz, 1H), 7.32-7.22 (m, 4H), 6.80 (d, J=8.2 Hz, 1H), 3.56 (s, 2H), 3.00 (t, J=5.9 Hz, 2H), 2.69 (t, J=5.9 Hz, 2H), 2.36 (s, 3H).

### Example 12. Synthesis of 7-(2-(4-fluorophenyl)-5-(1-methylpiperidin-4-yl)-1H-pyrrol-3-yl)tetrazolo[1,5-a]pyridine

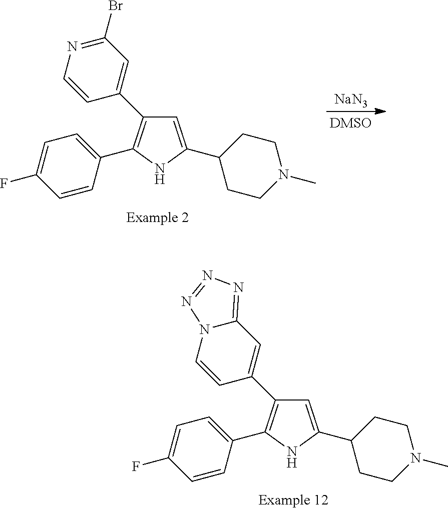 [SMILES: CN1CCC(c2cc(-c3ccnc(Br)c3)c(-c3ccc(F)cc3)[nH]2)CC1 ; CN1CCC(c2cc(-c3ccn4nnnc4c3)c(-c3ccc(F)cc3)[nH]2)CC1 ; "NaN3"]

A mixture of Example 2 (60 mg, 0.145 mmol, 1 eq) and NaN₃ (94 mg, 1.45 mmol, 10 eq) in DMSO (5 mL) was heated in a microwave reactor for 20 min at 200° C. The reaction was determined complete by LC/MS and partitioned in EtOAc/water. The organic was washed 3× water, 1× brine, dried over Na₂SO₄, filtered and concentrated. The crude residue after workup was purified by silica gel chromatography (0%→5%→10% MeOH/DCM with 1% 7N NH₃ in MeOH as a modifier). The product containing fractions were combined, concentrated and recrystallized from isopropanol to give the title compound Example 12 (15.1 mg, 0.040 mmol, 99% purity by HPLC) as alight yellow solid. MS: 376.90 [M+H]⁺. ¹H NMR (400 MHz, DMSO-d6) δ 11.25 (d, J=2.6 Hz, 1H), 9.05 (dd, J=7.2, 0.9 Hz, 1H), 7.83 (dd, J=1.7, 0.9 Hz, 1H), 7.44-7.36 (m, 2H), 7.28-7.17 (m, 2H), 7.08 (dd, J=7.2, 1.7 Hz, 1H), 6.31 (dd, J=2.7, 0.8 Hz, 1H), 2.83 (dt, J=11.6, 3.1 Hz, 2H), 2.54-2.50 (m, 1H), 2.17 (s, 3H), 1.99-1.88 (m, 4H), 1.66 (qd, J=12.4, 3.7 Hz, 3H).

### Example 13. Synthesis of 4-(2-(4-fluorophenyl)-5-(1-methylpiperidin-4-yl)-1H-pyrrol-3-yl)nicotinamide

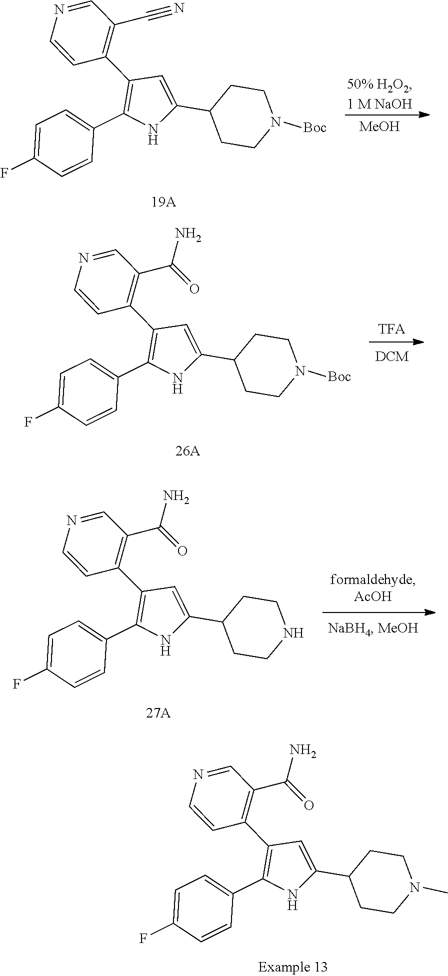 [SMILES: "MeOH" ; CC(C)(C)OC(=O)N1CCC(c2cc(-c3ccncc3C#N)c(-c3ccc(F)cc3)[nH]2)CC1 ; CC(C)(C)OC(=O)N1CCC(c2cc(-c3ccncc3C(N)=O)c(-c3ccc(F)cc3)[nH]2)CC1 ; NC(=O)c1cnccc1-c1cc(C2CCNCC2)[nH]c1-c1ccc(F)cc1 ; CN1CCC(c2cc(-c3ccncc3C(N)=O)c(-c3ccc(F)cc3)[nH]2)CC1]

### a. tert-Butyl 4-(4-(3-carbamoylpyridin-4-yl)-5-(4-fluorophenyl)-1H-pyrrol-2-yl)piperidine-1-carboxylate (26A)

To a solution of tert-butyl 4-(4-(3-cyanopyridin-4-yl)-5-(4-fluorophenyl)-1H-pyrrol-2-yl)piperidine-1-carboxylate (19A) (50 mg, 0.112 mmol, 1 eq) in DMSO (5 mL) was added NaOH (1M aq., 1 mL) followed by H₂O₂ (50% aq., 100 μL). After stirring for 10 minutes at room temperature, the reaction was determined complete by LC/MS and diluted with water up to 10 ml. The white solid that precipitated was sonicated, filtered, washed with water and dried under vacuum. The solid was recrystallized from a mixture of isopropanol/hexanes to afford (26A) (41 mg, 0.088 mmol, 92% purity by HPLC) as a white solid that was used without further purification. MS: 465.35 [M+H]⁺.

### b. 4-(2-(4-Fluorophenyl)-5-(piperidin-4-yl)-1H-pyrrol-3-yl)nicotinamide (27A)

To a solution of tert-butyl 4-(4-(3-carbamoylpyridin-4-yl)-5-(4-fluorophenyl)-1H-pyrrol-2-yl)piperidine-1-carboxylate (26A) (41 mg, 0.088 mmol, 1 eq) in DCM (2 mL) was added TFA (1 mL). The reaction was stirred for 30 min at room temperature, determined complete by LC/MS and concentrated to give (27A) (quantitative yield) that was used in the next reaction without further purification. MS: 364.65 [M+H]⁺.

### c. 4-(2-(4-Fluorophenyl)-5-(1-methylpiperidin-4-yl)-1H-pyrrol-3-yl)nicotinamide

Following the procedure for the synthesis of (10A) using (27A) (32 mg, 0.088 mmol, 1 eq), the title compound Example 13 (32 mg, 0.085 mmol, 98% purity by HPLC) was isolated as a yellow solid. MS: 379.0 [M+H]⁺. ¹H NMR (400 MHz, DMSO-d6) δ 11.05 (s, 1H), 8.46 (d, J=0.7 Hz, 1H), 8.27 (dd, J=5.2, 2.9 Hz, 1H), 7.63 (d, J=1.9 Hz, 1H), 7.43 (d, J=2.0 Hz, 1H), 7.34-7.26 (m, 2H), 7.17-7.08 (m, 2H), 6.86 (ddd, J=11.5, 5.2, 0.7 Hz, 1H), 5.96 (dd, J=10.7, 2.2 Hz, 1H), 2.89 (d, J=10.7 Hz, 2H), 2.24 (s, 3H), 2.07 (s, 2H), 1.91 (d, J=12.3 Hz, 2H), 1.62 (dd, J=13.8, 10.3 Hz, 2H).

### Example 14. Synthesis of 4-(2-(4-fluorophenyl)-1-methyl-5-(1-methylpiperidin-4-yl)-1H-pyrrol-3-yl)pyridine (34)

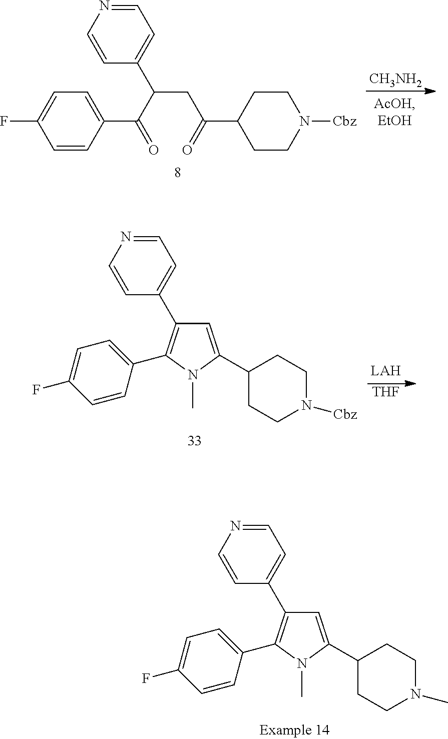 [SMILES: O=C(CC(C(=O)c1ccc(F)cc1)c1ccncc1)C1CCN(C(=O)OCc2ccccc2)CC1 ; "CH3NH2" ; Cn1c(C2CCN(C(=O)OCc3ccccc3)CC2)cc(-c2ccncc2)c1-c1ccc(F)cc1 ; CN1CCC(c2cc(-c3ccncc3)c(-c3ccc(F)cc3)n2C)CC1 ; "THF"]

### a. Benzyl 4-(5-(4-fluorophenyl)-1-methyl-4-(pyridin-4-yl)-1H-pyrrol-2-yl)piperidine-1-carboxylate (33)

Crude benzyl 4-(4-(4-fluorophenyl)-4-oxo-3-(pyridin-4-yl)butanoyl)piperidine-1-carboxylate (8) (200 mgs) was dissolved in 10 mL AcOH and 5 mL EtOH. Methylamine 2M solution in THF (12 mL) was added and the reaction was heated to reflux. After 3 hours, the reaction was determined complete by LC/MS analysis and was concentrated under reduced pressure. The concentrate was partitioned between EtOAc and water and the aqueous phase was extracted with EtOAc (3×). The combined organic phase was dried over sodium sulphate and concentrated. Purification by silica gel chromatography (10%→25%→50% EtOAc/Hexanes) afforded benzyl 4-(5-(4-fluorophenyl)-1-methyl-4-(pyridin-4-yl)-1H-pyrrol-2-yl)piperidine-1-carboxylate (33) (160 mgs, 65% yield, 64% purity by LC/MS) as a dark yellow solid. (M+H)+470.0, (M+Na)+492.0.

### b. 4-(2-(4-fluorophenyl)-1-methyl-5-(1-methylpiperidin-4-yl)-1H-pyrrol-3-yl)pyridine

Following the procedure as described for the synthesis of 4-(2-(4-fluorophenyl)-5-(1-methylpiperidin-4-yl)-1H-pyrrol-3-yl)pyridine (10), using benzyl 4-(5-(4-fluorophenyl)-1-methyl-4-(pyridin-4-yl)-1H-pyrrol-2-yl)piperidine-1-carboxylate (33) (160 mgs, 0.341 mmol, 1 eq), and LAH 2M solution in THF (0.68 mL, 1.36 mmol, 4 eq), the title compound was synthesized. Purified using silica gel chromatography (0%→1%→2% MeOH/EtOAc with 0.25% NH₃). Traces of compound (10) were still seen as was present in the crude product. Purification by reverse phase chromatography gave the bis HCl salt of the title compound. The salt was dissolved in 1N NaOH and partitioned between EtOAc and water. The aqueous phase was extracted with EtOAc (3×). The organic phase was combined, dried over sodium sulphate and concentrated to give the title compound Example 14 4-(2-(4-fluorophenyl)-1-methyl-5-(1-methylpiperidin-4-yl)-1H-pyrrol-3-yl)pyridine (20 mgs, 27% yield, 100% purity by LC/MS) as a pale-yellow solid. ¹H NMR (400 MHz, DMSO-d₆) δ 8.23 (d, J=5.4 Hz, 2H), 7.37-7.25 (m, 4H), 6.97-6.92 (m, 2H), 6.28 (d, J=0.6 Hz, 1H), 2.85 (dt, J=11.9, 3.0 Hz, 2H), 2.55 (ddt, J=11.7, 8.6, 3.6 Hz, 1H), 2.19 (s, 3H), 2.05-1.93 (m, 2H), 1.86 (ddd, J=13.2, 3.5, 1.6 Hz, 2H), 1.62 (qd, J=12.5, 3.7 Hz, 2H). (M+H)+ 349.85.

As the pyrrole N-Me protons were obscured by a water signal, the bis HCl salt of the title compound was prepared to view all the proton signals. ¹H NMR (400 MHz, DMSO-d₆) δ 11.02 (s, 1H), 8.53-8.45 (m, 2H), 7.54-7.35 (m, 6H), 6.67 (s, 1H), 3.47 (d, J=12.6 Hz, 2H), 3.35 (s, 3H), 3.07 (dt, J=13.4, 10.3 Hz, 2H), 2.96 (td, J=11.7, 5.8 Hz, 1H), 2.72 (d, J=4.5 Hz, 3H), 2.16-1.93 (m, 4H).

### Example 15. Synthesis of 4-(4-bromo-2-(4-fluorophenyl)-5-(1-methylpiperidin-4-yl)-1H-pyrrol-3-yl)pyridine (11)

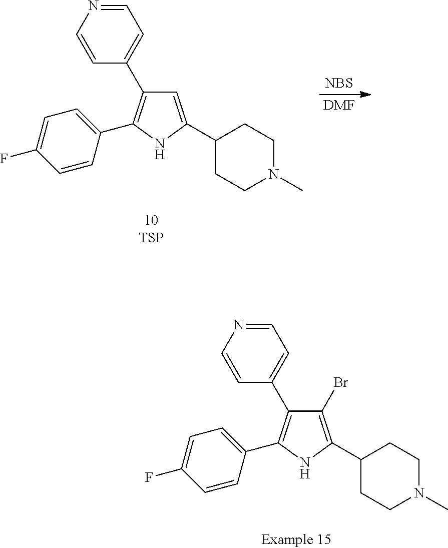

4-(2-(4-Fluorophenyl)-5-(1-methylpiperidin-4-yl)-1H-pyrrol-3-yl)pyridine (10) (1.5 g, 4.47 mmol, 1 eq) was dissolved in 20 mL anhydrous DMF and cooled to 0° C. NBS (637 mgs, 3.58 mmol, 0.8 eq) was added portion wise to the solution of (10) over 5 minutes and the reaction was stirred maintaining the temperature at 0° C. The reaction reached completion in 1 hour as determined by LC/MS analysis. The product immediately precipitated out after addition of ice cold water. The reaction mixture was filtered and the residue was collected. Purification by recrystallization using MeOH afforded the title compound Example 15 4-(4-bromo-2-(4-fluorophenyl)-5-(1-methylpiperidin-4-yl)-1H-pyrrol-3-yl)pyridine (1.45 g, 78% yield, 99% purity by LC/MS) as an off-white solid. ¹H NMR (400 MHz, DMSO-d₆) δ 11.41 (s, 1H), 8.50-8.42 (m, 2H), 7.22-7.10 (m, 6H), 2.87 (d, J=6.3 Hz, 2H), 2.69 (dd, J=8.2, 4.1 Hz, 1H), 2.19 (s, 3H), 2.03-1.86 (m, 4H), 1.67 (p, J=5.2, 4.0 Hz, 2H). (M+H)+ 413.75, 415.80.

### Example 16. Synthesis of Ethyl 5-(4-fluorophenyl)-2-(1-methylpiperidin-4-yl)-4-(pyridin-4-yl)-1H-pyrrole-3-carboxylate

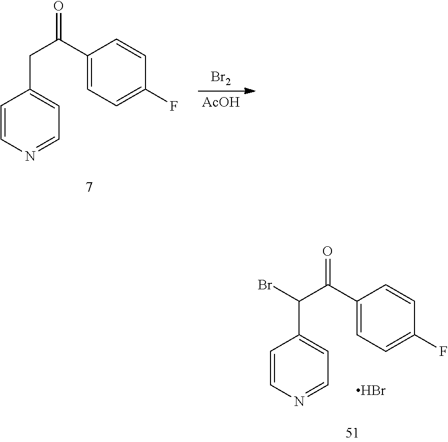

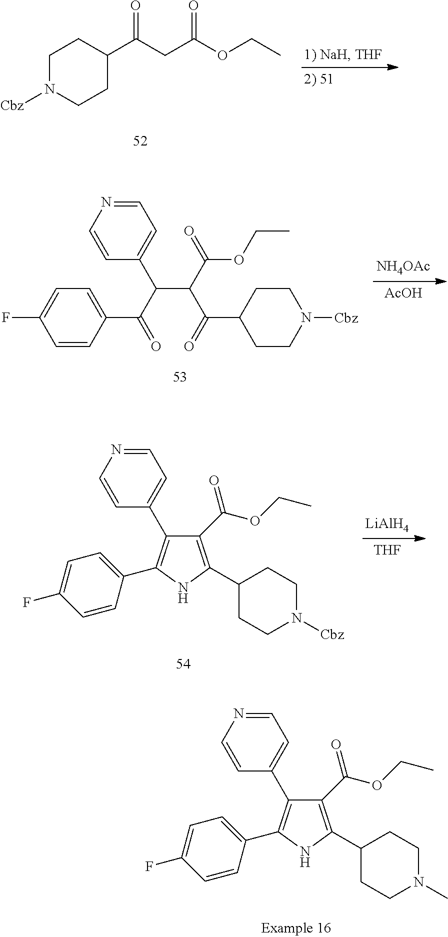

### a. 2-Bromo-1-(4-fluorophenyl)-2-(pyridin-4-yl)ethan-1-one hydrobromide (51)

1-(4-Fluorophenyl)-2-(pyridin-4-yl)ethan-1-one (7) (1.274 g, 5.92 mmol, 1 eq) was dissolved in 10 mL AcOH. A stock solution of bromine (2.12 g, 0.6 mL, density=3.1 g/mL) in 2 mL AcOH was prepared. 1.045 mL of the stock solution of bromine (amounting to 852 mgs, 5.328 mmol, 0.9 eq of Br₂) was added to the solution of (7) in a dropwise manner by means of a syringe. The reaction was stirred at room temperature for 45 minutes which resulted in complete bromination of (7) as determined by the LC/MS. The hydrobromide salt of the product was seen to crash out of the solution. 3 mL EtOAc was added and stirred for 10 minutes to achieve total precipitation of the product. The reaction mixture was filtered, and the residue was dried and collected to give 2-bromo-1-(4-fluorophenyl)-2-(pyridin-4-yl)ethan-1-one hydrobromide (51) (1.88 g, 85% yield, 98% purity by LC/MS) as a beige-yellow crystalline solid which was taken onto the next step without any purification. (M+H)+ 294.95, 295.95.

### b. Benzyl 4-(2-(ethoxycarbonyl)-4-(4-fluorophenyl)-4-oxo-3-(pyridin-4-yl)butanoyl)piperidine-1-carboxylate (53)

Benzyl 4-(3-ethoxy-3-oxopropanoyl)piperidine-1-carboxylate (52) (1.1 g, 3.299 mmol, 1 eq) was dissolved in 14 mL THF. NaH 60% dispersion in mineral oil (237.48 mgs, 9.899 mmol, 3 eq) and 2-bromo-1-(4-fluorophenyl)-2-(pyridin-4-yl)ethan-1-one hydrobromide (51) (1.234 g, 3.299 mmol, 1 eq) were added in a sequential manner and the reaction was stirred overnight. LC/MS analysis revealed that the reaction was complete, and 3 mL water was added to quench the reaction. The reaction was concentrated under reduced pressure and the concentrate was partitioned between EtOAc and water. The aqueous phase was extracted with EtOAc (3×) and the combined organic phase was dried over sodium sulphate and concentrated to give crude benzyl 4-(2-(ethoxycarbonyl)-4-(4-fluorophenyl)-4-oxo-3-(pyridin-4-yl)butanoyl)piperidine-1-carboxylate (53) (1.3 g) which was taken onto the next step without any purification. (M+H)+547.10.

### c. Benzyl 4-(3-(ethoxycarbonyl)-5-(4-fluorophenyl)-4-(pyridin-4-yl)-1H-pyrrol-2-yl)piperidine-1-carboxylate (54)

Following the procedure as described for the synthesis of benzyl 4-(5-(4-fluorophenyl)-4-(pyridin-4-yl)-1H-pyrrol-2-yl)piperidine-1-carboxylate (9) using crude benzyl 4-(2-(ethoxycarbonyl)-4-(4-fluorophenyl)-4-oxo-3-(pyridin-4-yl)butanoyl)piperidine-1-carboxylate (53) (1.3 g) and NH₄OAc (3 g), the compound (54) was synthesized. Purification by silica gel chromatography (50% EtOAc/Hexanes) afforded benzyl 4-(3-(ethoxycarbonyl)-5-(4-fluorophenyl)-4-(pyridin-4-yl)-1H-pyrrol-2-yl)piperidine-1-carboxylate (54) (600 mg, 97% purity by LC/MS). (M+H)+ 527.95.

### d. Ethyl 5-(4-fluorophenyl)-2-(1-methylpiperidin-4-yl)-4-(pyridin-4-yl)-1H-pyrrole-3-carboxylate

Following the procedure as described for the synthesis of 4-(2-(4-fluorophenyl)-5-(1-methylpiperidin-4-yl)-1H-pyrrol-3-yl)pyridine (10) using benzyl 4-(3-(ethoxycarbonyl)-5-(4-fluorophenyl)-4-(pyridin-4-yl)-1H-pyrrol-2-yl)piperidine-1-carboxylate (54) (358 mg, 0.679 mmol, 1 eq) and LAH 2M solution in THF (1.35 mL, 2.7 mmol, 4 eq), the title compound was synthesized without any reduction of the ester moiety. Purification by silica gel chromatography (8% MeOH/CHCl₃ with 0.25% NH₃) afforded the title compound Example 16 ethyl 5-(4-fluorophenyl)-2-(1-methylpiperidin-4-yl)-4-(pyridin-4-yl)-1H-pyrrole-3-carboxylate (121 mg, 44% yield, 100% purity by LC/MS) as a clear white solid. ¹H NMR (400 MHz, DMSO-d₆) δ 11.44 (s, 1H), 8.41-8.37 (m, 2H), 7.19-7.12 (m, 2H), 7.12-7.05 (m, 4H), 3.94 (q, J=7.1 Hz, 2H), 3.35 (td, J=8.9, 4.1 Hz, 1H), 2.93-2.83 (m, 2H), 2.18 (s, 3H), 2.06-1.84 (m, 4H), 1.70 (dd, J=9.6, 3.8 Hz, 2H), 0.92 (t, J=7.1 Hz, 3H). (M+H)+ 408.10.

### Example 17. Synthesis of 4-(2-(4-fluorophenyl)-5-(1-methylpiperidin-4-yl)-4-vinyl-1H-pyrrol-3-yl)pyridine (12)

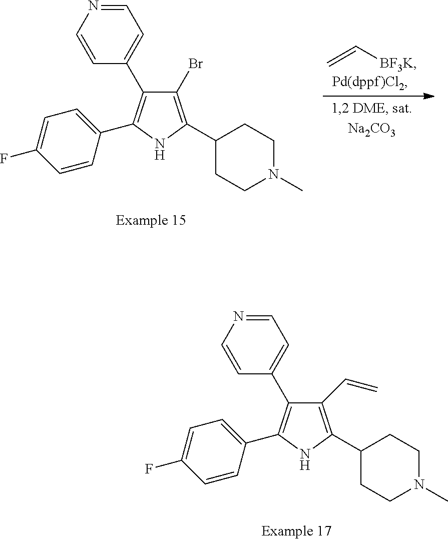

### a. 4-(2-(4-Fluorophenyl)-5-(1-methylpiperidin-4-yl)-4-vinyl-1H-pyrrol-3-yl)pyridine

Example 15, 4-(4-Bromo-2-(4-fluorophenyl)-5-(1-methylpiperidin-4-yl)-1H-pyrrol-3-yl)pyridine (224 mgs, 0.542 mmol, 1 eq), potassium vinyltrifluoroborate (145 mgs, 1.084 mmol, 2 eq) and Pd(dppf)Cl₂ (44 mgs, 0.054 mmol, 0.1 eq) were dissolved in 4 mL 1,2 DME in a 5 mL microwave vial. Saturated Na₂CO₃ (1 mL) was added, the vial was sealed shut and heated in the microwave at 120° C. After 30 minutes the reaction was determined complete by LC/MS analysis. The reaction mixture was filtered over celite and the filtrate was concentrated. Purification by silica gel chromatography (2%→5%→10% EtOAc/MeOH with 0.25%1 NH₃) was carried out. A second purification by silica gel chromatography (4%→8% MeOH/CHCl₃ with 0.25% NH₃) afforded the title compound Example 17 4-(2-(4-fluorophenyl)-5-(1-methylpiperidin-4-yl)-4-vinyl-1H-pyrrol-3-yl)pyridine (90 mgs, 46% yield, 98% purity by LC/MS) as a yellowish-white solid. ¹H NMR (400 MHz, DMSO-d₆) δ 11.03 (s, 1H), 8.48-8.40 (m, 2H), 7.19-7.12 (m, 2H), 7.12-7.05 (m, 4H), 6.46 (dd, J=17.8, 11.5 Hz, 1H), 4.94 (dd, J=11.5, 2.0 Hz, 1H), 4.80 (dd, J=17.8, 2.1 Hz, 1H), 2.91-2.82 (m, 2H), 2.76 (dt, J=11.7, 6.1 Hz, 1H), 2.19 (s, 3H), 2.05-1.87 (m, 4H), 1.65 (d, J=9.8 Hz, 2H). (M+H)+ 361.65.

### Example 18. Synthesis of 5-(4-fluorophenyl)-2-(1-methylpiperidin-4-yl)-4-(pyridin-4-yl)-1H-pyrrole-3-carbonitrile

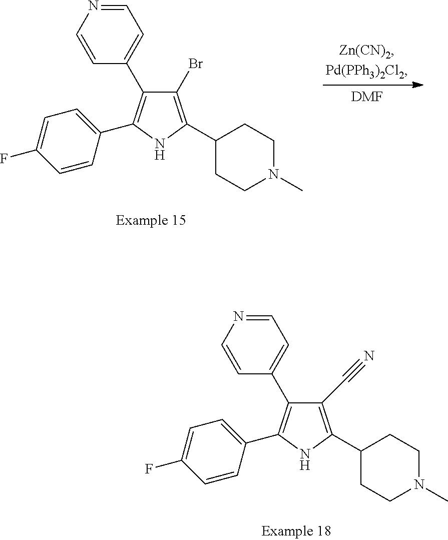

Example 15, 4-(4-bromo-2-(4-fluorophenyl)-5-(1-methylpiperidin-4-yl)-1H-pyrrol-3-yl)pyridine (110 mgs, 0.267 mmol, 1 eq) and Zn(CN)₂ (624 mgs, 5.31 mmol, 20 eq) were dissolved in 6 mL anhydrous DMF in a microwave vial. The solution was degassed under N₂ for 10 minutes. Pd(PPh₃)₂Cl₂ (37 mgs, 0.05 mmol, 0.19 eq) was added and the reaction was heated in the microwave at 180° C. After 1.5 hours, the reaction was determined complete by LC/MS analysis. The reaction was partitioned between water and EtOAc and the aqueous phase was extracted with EtOAc (3×). The combined organic phase was washed with brine (1×), dried over sodium sulphate and concentrated. Excess DMF was chased by forming an azeotropic mixture with toluene. The resulting crude product was purified by silica gel chromatography (1%→2%→4% EtOAc/MeOH with 0.25% NH₃). A second purification by silica gel chromatography (8% MeOH/CHCl₃ with 0.25% NH₃) afforded the title compound Example 18 5-(4-fluorophenyl)-2-(1-methylpiperidin-4-yl)-4-(pyridin-4-yl)-1H-pyrrole-3-carbonitrile (52 mgs, 54% yield, 97% purity by LC/MS) as a crystalline yellow solid. ¹H NMR (400 MHz, DMSO-d₆) δ 12.03 (s, 1H), 8.53-8.47 (m, 2H), 7.32-7.26 (m, 2H), 7.23-7.16 (m, 4H), 2.88 (dd, J=7.1, 2.0 Hz, 2H), 2.81-2.69 (m, 1H), 2.19 (s, 3H), 2.01-1.88 (m, 4H), 1.87-1.75 (m, 2H). (M+H)+ 361.05.

### Example 19. Synthesis of 5-(4-fluorophenyl)-2-(1-methylpiperidin-4-yl)-4-(pyridin-4-yl)-1H-pyrrole-3-carboxamide

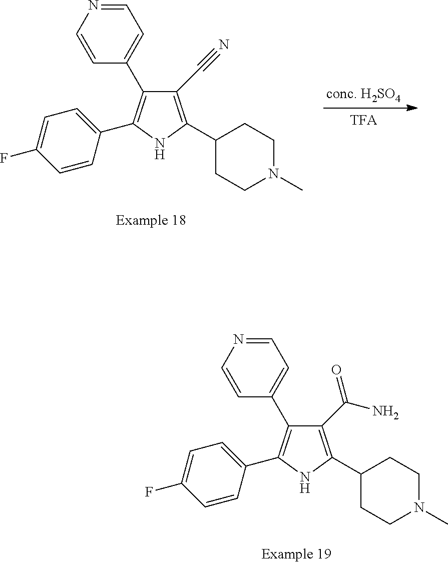 [SMILES: CN1CCC(c2[nH]c(-c3ccc(F)cc3)c(-c3ccncc3)c2C#N)CC1 ; "conc. H2SO4" ; CN1CCC(c2[nH]c(-c3ccc(F)cc3)c(-c3ccncc3)c2C(N)=O)CC1 ; "TFA"]

Example 18, 5-(4-fluorophenyl)-2-(1-methylpiperidin-4-yl)-4-(pyridin-4-yl)-1H-pyrrole-3-carbonitrile (51 mgs, 0.142 mmol, 1 eq) was dissolved in 3.5 mL TFA in a microwave vial. Concentrated H₂SO₄ (0.4 mL) and water (0.1 mL) was added and the reaction was heated in the microwave at 100° C. for 1.5 hours. Total conversion to the primary amide was observed by LC/MS analysis. The reaction was poured into ice-cold water and 1M NaOH was added until the solution was basic. The product precipitated out and the precipitate was collected by filtration. The filtrate was extracted with EtOAc (2×), dried over sodium sulphate, concentrated and combined with the precipitate from the filtration. Purification by silica gel chromatography (10%→20% (25% MeOH/AcCN)/DCM with 0.25% NH₃) was carried out. A second purification by silica gel chromatography (10%→15%→20% MeOH/EtOAc with 0.25% NH₃) afforded the title compound Example 19 5-(4-fluorophenyl)-2-(1-methylpiperidin-4-yl)-4-(pyridin-4-yl)-1H-pyrrole-3-carboxamide (20 mgs, 37% yield, 95% purity by LC/MS) as a crystalline yellow solid. ¹H NMR (400 MHz, DMSO-dt) δ 11.18 (s, 1H), 8.40-8.37 (m, 2H), 7.23-7.11 (m, 4H), 7.11-7.08 (m, 2H), 7.01 (s, 1H), 6.87 (s, 1H), 2.92-2.81 (m, 3H), 2.18 (s, 3H), 1.99-1.86 (m, 4H), 1.73 (d, J=11.1 Hz, 2H). (M+H)+ 379.05.

### Example 20. Synthesis of 1-(5-(4-fluorophenyl)-2-(1-methylpiperidin-4-yl)-4-(pyridin-4-yl)-1H-pyrrol-3-yl)ethan-1-ol

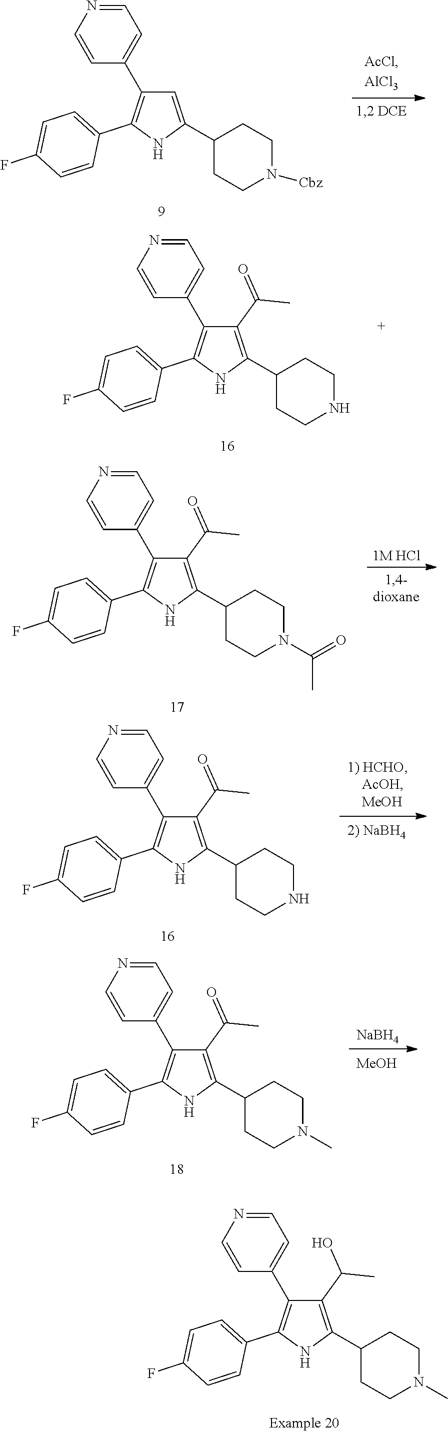

### a. 1-(5-(4-Fluorophenyl)-2-(piperidin-4-yl)-4-(pyridin-4-yl)-1H-pyrrol-3-yl)ethan-1-one (16)

Acetyl chloride (0.02 mL, density=1.104 g/mL, 0.296 mmol, 1.5 eq) was dissolved in 2 mL 1,2 DCE and cooled to 0° C. under N₂. AlCl₃ (40 mgs, 0.296 mmol, 1.5 eq) was added to the AcCl solution and stirred for 20 minutes while maintaining the temperature at 0° C. Benzyl 4-(5-(4-fluorophenyl)-4-(pyridin-4-yl)-1H-pyrrol-2-yl)piperidine-1-carboxylate (9) (90 mgs, 0.198 mmol, 1 eq) was dissolved in 5 mL 1,2 DCE and added to the reaction mixture by means of a syringe. The reaction was warmed to room temperature and stirred overnight. A mixture of products 16 and 17 was observed by LC/MS analysis. The reaction was quenched by addition of ice-cold water. The reaction mixture was partitioned between water and DCM and the organic phase was extracted with water (3×). The combined aqueous phase was basified by addition of 1N NaOH and partitioned between water and DCM. The aqueous phase was extracted with DCM (3×). The combined organic phase was dried over sodium sulphate and concentrated to give a crude mixture of 16 and 17. The crude mixture was dissolved in 2 mL 1,4-dioxane in a sealed vial and concentrated HCl (1 mL) was added. The reaction was heated to reflux for 3 hours and was determined complete by LC/MS analysis. The reaction mixture was basified by addition of 1N NaOH and partitioned between water and EtOAc. The aqueous phase was extracted with EtOAc (3×). The combined organic phase was dried over celite and concentrated to give 1-(5-(4-fluorophenyl)-2-(piperidin-4-yl)-4-(pyridin-4-yl)-1H-pyrrol-3-yl)ethan-1-one (16) (56 mgs, 79% yield over 2 steps, 84% purity by LC/MS) which was taken onto the next step without any purification. (M+H)+ 363.85.

### b. 1-(5-(4-Fluorophenyl)-2-(1-methylpiperidin-4-yl)-4-(pyridin-4-yl)-1H-pyrrol-3-yl)ethan-1-one (18)

1-(5-(4-Fluorophenyl)-2-(piperidin-4-yl)-4-(pyridin-4-yl)-1H-pyrrol-3-yl)ethan-1-one (16) (56 mgs, 0.154 mmol, 1 eq) was dissolved in 5 mL MeOH and 1 mL AcOH. Formaldehyde 37% w/w in H₂O (1 mL) was added to the solution of 16. The reaction was stirred at room temperature for 1 hour. Sodium borohydride (50 mgs, 1.31 mmol, 8.5 eq) was added portion wise to the reaction mixture over 10 minutes. After 1 hour, the reaction was determined complete by LC/MS analysis. The reaction was quenched by addition of 2 mL water and was concentrated under reduced pressure. The concentrate was partitioned between EtOAc and water. The aqueous phase was extracted with EtOAc (3×). The combined organic phase was dried over sodium sulphate and concentrated to give 1-(5-(4-fluorophenyl)-2-(1-methylpiperidin-4-yl)-4-(pyridin-4-yl)-1H-pyrrol-3-yl)ethan-1-one (18) (55 mgs, 95% yield, 86% purity by LC/MS) which was taken onto the next step without any purification. (M+H)+378.25.

### c. 1-(5-(4-Fluorophenyl)-2-(1-methylpiperidin-4-yl)-4-(pyridin-4-yl)-1H-pyrrol-3-yl)ethan-1-ol

To a solution of 1-(5-(4-fluorophenyl)-2-(1-methylpiperidin-4-yl)-4-(pyridin-4-yl)-1H-pyrrol-3-yl)ethan-1-one (18) (55 mgs, 0.146 mmol, 1 eq) in 7 mL MeOH, sodium borohydride (50 mgs, 1.322 mmol, 9 eq) was added portion wise. The reaction was stirred at room temperature over the weekend during which it achieved completion as determined by LC/MS analysis. 2 mL water was added to quench the reaction and it was concentrated under reduced pressure. The concentrate was partitioned between water and EtOAc and the aqueous phase was extracted with EtOAc (3×). The combined organic phase was dried over sodium sulphate and concentrated. Purification by silica gel chromatography (8% MeOH/CHCl₃ with 0.25% NH₃) afforded the title compound Example 201-(5-(4-fluorophenyl)-2-(1-methylpiperidin-4-yl)-4-(pyridin-4-yl)-1H-pyrrol-3-yl)ethan-1-ol (30 mgs, 55% yield, 95% purity by LC/MS) as a deep yellow solid. ¹H NMR (400 MHz, DMSO-d₆) δ 10.78 (s, 1H), 8.44-8.39 (m, 2H), 7.17-7.14 (m, 2H), 7.14-7.02 (m, 4H), 4.69 (d, J=2.6 Hz, 1H), 4.63 (qd, J=6.6, 2.7 Hz, 1H), 2.99 (d, J=11.6 Hz, 1H), 2.87 (d, J=6.7 Hz, 2H), 2.19 (s, 3H), 1.93 (p, J=10.9, 10.4 Hz, 4H), 1.74-1.59 (m, 2H), 1.20 (d, J=6.6 Hz, 3H).

### Example 21. Synthesis of 5-(4-fluorophenyl)-2-(1-methylpiperidin-4-yl)-4-(pyridin-4-yl)-1H-pyrrol-3-yl)methanol

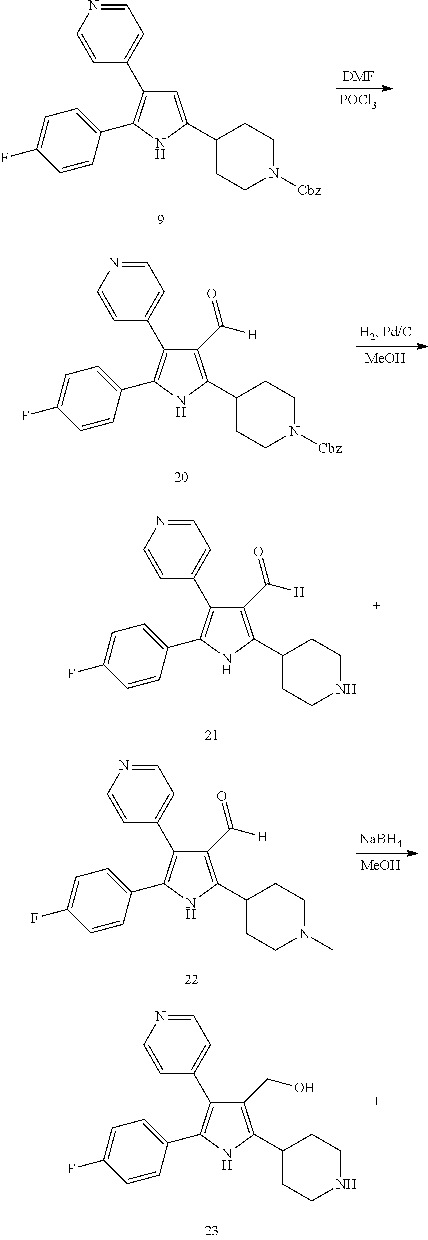

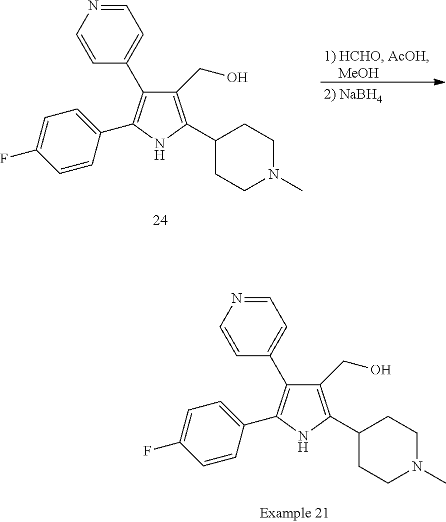

### a. Benzyl 4-(5-(4-fluorophenyl) 3-formyl-4-(pyridin-4-yl)-1H-pyrrol-2-yl)piperidine-1-carboxylate (20)

Anhydrous DMF (5 mL) was cooled to 0° C. under N₂ and POCl₃ (0.2 mL) was added to it. The reaction was stirred for 45 minutes. Benzyl 4-(5-(4-fluorophenyl)-4-(pyridin-4-yl)-1H-pyrrol-2-yl)piperidine-1-carboxylate (9) (90 mgs, 0.198 mmol, 1 eq) was dissolved in 5 mL dry DMF and added to the Vilsmeier salt by means of a syringe. The reaction was warmed to 60° C. and the reaction was determined complete in 30 minutes by LC/MS analysis. The reaction was quenched by addition of 1N NaOH until basic and concentrated under reduced pressure. The concentrate was partitioned between water and EtOAc and the aqueous phase was extracted with EtOAc (3×). The combined organic phase was washed with brine, dried over sodium sulphate and concentrated. Excess DMF was chased by forming an azeotropic mixture with toluene. Purification by silica gel chromatography (25%→50% EtOAc/Hexanes) afforded benzyl 4-(5-(4-fluorophenyl)-3-formyl-4-(pyridin-4-yl)-1H-pyrrol-2-yl)piperidine-1-carboxylate (20) (60 mgs, 63% yield, 94% purity by LC/MS). (M+H)+ 483.95.

### b. 5-(4-Fluorophenyl)-2-(piperidin-4-yl)-4-(pyridin-4-yl)-1H-pyrrole-3-carbaldehyde (21) and 5-(4-fluorophenyl)-2-(1-methylpiperidin-4-yl)-4-(pyridin-4-yl)-1H-pyrrole-3-carbaldehyde (22)

Benzyl 4-(5-(4-fluorophenyl)-3-formyl-4-(pyridin-4-yl)-1H-pyrrol-2-yl)piperidine-1-carboxylate (20) (60 mgs, 0.124 mmol, 1 eq) was dissolved in 10 mL MeOH. Palladium on carbon (50 mgs) was added to the methanolic solution of 20. The reaction mixture was set up for hydrogenation using a hydrogen balloon setup. The reaction was stirred for 3 hours at room temperature and was determined complete by LC/MS analysis. The reaction mixture was filtered over celite and the filtrate was concentrated to give a 1:1 mixture of 5-(4-fluorophenyl)-2-(piperidin-4-yl)-4-(pyridin-4-yl)-1H-pyrrole-3-carbaldehyde (21) and 5-(4-fluorophenyl)-2-(1-methylpiperidin-4-yl)-4-(pyridin-4-yl)-1H-pyrrole-3-carbaldehyde (22) (65 mgs) which was taken onto the next step without any purification. (M+H)+ 349.95, 363.85.

### c. (5-(4-Fluorophenyl)-2-(piperidin-4-yl)-4-(pyridin-4-yl)-1H-pyrrol-3-yl)methanol (23) and (5-(4-fluorophenyl)-2-(1-methylpiperidin-4-yl)-4-(pyridin-4-yl)-1H-pyrrol-3-yl)methanol (24)

A 1:1 mixture of 5-(4-fluorophenyl)-2-(piperidin-4-yl)-4-(pyridin-4-yl)-1H-pyrrole-3-carbaldehyde (21) and 5-(4-fluorophenyl)-2-(1-methylpiperidin-4-yl)-4-(pyridin-4-yl)-1H-pyrrole-3-carbaldehyde (22) (65 mgs) was dissolved in 8 mL MeOH and sodium borohydride (50 mgs) was added to this. The reaction was determined complete by LC/MS analysis after stirring for 1 hour. 4 mL water was added to quench the reaction and it was concentrated under reduced pressure. The concentrate was partitioned between EtOAc and water and the aqueous phase was extracted with EtOAc (3×). The combined organic phase was dried over sodium sulphate and concentrated to give a mixture of (5-(4-fluorophenyl)-2-(piperidin-4-yl)-4-(pyridin-4-yl)-1H-pyrrol-3-yl)methanol (23) and (5-(4-fluorophenyl)-2-(1-methylpiperidin-4-yl)-4-(pyridin-4-yl)-1H-pyrrol-3-yl)methanol (24) (50 mgs) which was taken onto the next step without any purification. (M+H)+ 351.65, 365.75.

### d. (5-(4-fluorophenyl)-2-(1-methylpiperidin-4-yl)-4-(pyridin-4-yl)-H-pyrrol-3-yl)methanol

A mixture of (5-(4-fluorophenyl)-2-(piperidin-4-yl)-4-(pyridin-4-yl)-1H-pyrrol-3-yl)methanol (23) and (5-(4-fluorophenyl)-2-(1-methylpiperidin-4-yl)-4-(pyridin-4-yl)-1H-pyrrol-3-yl)methanol (24) (50 mgs) was taken up in 8 mL MeOH and 2 mL AcOH. Formaldehyde 37% w/w solution (1 mL) was added to this solution and the reaction was stirred at room temperature for 1 hour. Sodium borohydride (35 mgs) was added to the reaction mixture and the reaction stirred over the weekend to achieve completion. 1 mL water was added to quench the reaction and it was concentrated under reduced pressure. The concentrate was partitioned between EtOAc and water and the aqueous phase was extracted with EtOAc (3×). The combined organic phase was dried over sodium sulphate and concentrated. New mass peak corresponding to possible hydroxymethylation of the piperidine nitrogen was seen. The crude mixture was taken up in 5 mL 7N NH₃ solution in MeOH and stirred overnight to give total conversion to the desired product. The reaction was concentrated under reduced pressure. Purification by silica gel chromatography (8%→10% MeOH/CHCl₃ with 0.25% NH₃) afforded the title compound Example 21 (5-(4-fluorophenyl)-2-(1-methylpiperidin-4-yl)-4-(pyridin-4-yl)-1H-pyrrol-3-yl)methanol (20 mgs, 100% purity by LC/MS) as a deep yellow solid. ¹H NMR (400 MHz, DMSO-4) δ 10.90 (s, 1H), 8.41-8.37 (m, 2H), 7.25-7.16 (m, 4H), 7.15-7.07 (m, 2H), 4.61-4.55 (m, 1H), 4.18 (d, J=4.7 Hz, 2H), 2.85 (d, J=6.8 Hz, 2H), 2.72 (dq, J=8.4, 4.2 Hz, 1H), 2.18 (s, 3H), 2.00-1.86 (m, 4H), 1.67 (q, J=4.7, 4.1 Hz, 2H). (M+H)+ 365.80.

### Example 22. Synthesis of (5-(4-fluorophenyl)-2-(1-methylpiperidin-4-yl)-4-(pyridin-4-yl)-1H-pyrrol-3-yl)-N,N-dimethylmethanamine

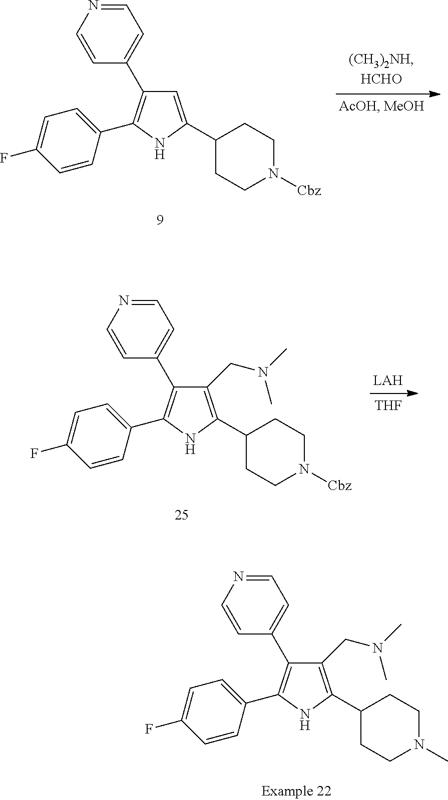

### a. Benzyl 4-(3-((dimethylamino)methyl)-5-(4-fluorophenyl)-4-(pyridin-4-yl)-1H-pyrrol-2-yl)piperidine-1-carboxylate (25)

Formaldehyde 37% w/w solution in water (1.7 mL) was taken up in 8 mL MeOH and 1 mL AcOH and dimethylamine 2M solution in THF (1.8 mL) was added to this. The reaction was stirred for 1 hour under N₂. Benzyl 4-(5-(4-fluorophenyl)-4-(pyridin-4-yl)-1H-pyrrol-2-yl)piperidine-1-carboxylate (9) (90 mgs, 0.198 mmol, 1 eq) was dissolved in 8 mL MeOH and added dropwise to the Mannich base generated by means of a syringe. The reaction was warmed to 60° C. The reaction was determined almost complete in 12 days and was found to progress ahead each day by LC/MS analysis. 1N NaOH was added until the reaction mixture turned basic and it was concentrated under reduced pressure. The concentrate was partitioned between water and EtOAc and the aqueous phase was extracted with EtOAc (3×). The combined organic phase was dried over sodium sulphate and concentrated. Purification by silica gel chromatography (1% 4 2%→4%→6%→8%→10% MeOH/DCM with 0.25% NH₃) gave benzyl 4-(3-((dimethylamino)methyl)-5-(4-fluorophenyl)-4-(pyridin-4-yl)-1H-pyrrol-2-yl)piperidine-1-carboxylate (25) (65 mgs, 64% yield, 97% purity by LC/MS). (M+H)+ 513.10.

### b. 1-(5-(4-Fluorophenyl)-2-(1-methylpiperidin-4-yl)-4-(pyridin-4-yl)-1H-pyrrol-3-yl)-N,N-dimethylmethanamine

Benzyl 4-(3-((dimethylamino)methyl)-5-(4-fluorophenyl)-4-(pyridin-4-yl)-1H-pyrrol-2-yl)piperidine-1-carboxylate (25) (65 mgs, 0.127 mmol, 1 eq) was dissolved in 6 mL anhydrous THF. Lithium aluminium hydride 2M solution in THF (0.25 mL, 0.508 mmol, 4 eq) was added dropwise to the solution of 25 and stirred at room temperature for 30 minutes. The reaction was determined complete by LC/MS analysis. The Feiser & Feiser quench (0.02 mL water, 0.02 mL 15% NaOH and 0.06 mL water in a sequential manner) and stirred for an additional 45 minutes. The reaction was filtered over celite and the filtrate was concentrated. Purification by silica gel chromatography (10%→12%→15% MeOH/EtOAc with 0.25% NH₃) fairly purified the crude product. The almost pure product was partitioned between 1N HCl and EtOAc and the organic phase was extracted with water (2×). The combined acidic aqueous phase was basified by addition of 1N NaOH till the pH was 9 and EtOAc was added. The neutralised aqueous phase was extracted with EtOAc (3×), dried over sodium sulphate and concentrated to give the title compound Example 22 1-(5-(4-fluorophenyl)-2-(1-methylpiperidin-4-yl)-4-(pyridin-4-yl)-1H-pyrrol-3-yl)-N,N-dimethylmethanamine (30 mgs, 60% yield, 98% purity by LC/MS) as a white solid. ¹H NMR (400 MHz, DMSO-d₆) δ 10.89 (s, 1H), 8.40-8.35 (m, 2H), 7.31-7.26 (m, 2H), 7.23-7.17 (m, 2H), 7.12-7.05 (m, 2H), 3.03 (s, 2H), 2.89-2.82 (m, 2H), 2.72-2.62 (m, 1H), 2.17 (s, 3H), 2.07 (s, 6H), 2.00-1.86 (m, 4H), 1.58 (d, J=13.5 Hz, 2H). (M+H)+ 393.25.

### Example 23. Synthesis of Benzyl 4-(5-(4-fluorophenyl)-3-((methylamino)methyl)-4-(pyridin-4-yl)-1H-pyrrol-2-yl)piperidine-1-carboxylate

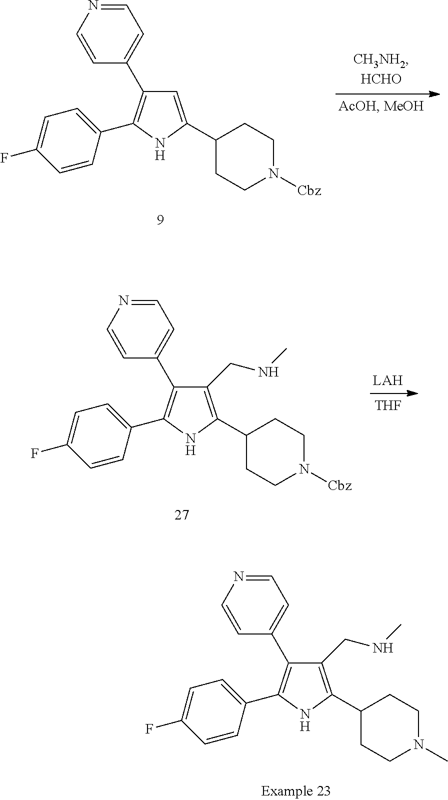

### a. Benzyl 4-(5-(4-fluorophenyl)-3-((methylamino)methyl)-4-(pyridin-4-yl)-1H-pyrrol-2-yl)piperidine-1-carboxylate (27)

Following the procedure as described for the synthesis of 25 using benzyl 4-(5-(4-fluorophenyl)-4-(pyridin-4-yl)-1H-pyrrol-2-yl)piperidine-1-carboxylate (9) (115 mgs, 0.252 mmol, 1 eq), methylamine 2M solution in THF (0.7 mL, 1.4 mmol, 5.5 eq) and formaldehyde 37% w/w solution in water (0.1 mL), the compound benzyl 4-(5-(4-fluorophenyl)-3-((methylamino)methyl)-4-(pyridin-4-yl)-1H-pyrrol-2-yl)piperidine-1-carboxylate (27) (65 mgs, 52% yield, 98% purity by LC/MS) was isolated. (M+H)+ 499.15.

### b. Benzyl 4-(5-(4-fluorophenyl)-3-((methylamino)methyl)-4-(pyridin-4-yl)-1H-pyrrol-2-yl)piperidine-1-carboxylate

Following the procedure as described for the synthesis of Example 22, using benzyl 4-(5-(4-fluorophenyl)-3-((methylamino)methyl)-4-(pyridin-4-yl)-1H-pyrrol-2-yl)piperidine-1-carboxylate (27) (60 mgs, 0.120 mmol, 1 eq) and lithium aluminium hydride 2M solution in THF (0.24 mL, 0.48 mmol, 4 eq), the title compound Example 23 benzyl 4-(5-(4-fluorophenyl)-3-((methylamino)methyl)-4-(pyridin-4-yl)-1H-pyrrol-2-yl)piperidine-1-carboxylate (15 mgs, 33% yield, 98% purity by LC/MS) was isolated as a crystalline white solid. ¹H NMR (400 MHz, DMSO-d₆) δ 10.89 (s, 1H), 8.42-8.38 (m, 2H), 7.27-7.23 (m, 2H), 7.20-7.16 (m, 2H), 7.12-7.06 (m, 2H), 3.36 (s, 2H), 2.87-2.82 (m, 2H), 2.71-2.63 (m, 1H), 2.25 (s, 3H), 2.18 (s, 3H), 1.97-1.87 (m, 4H), 1.64 (q. J=4.8, 3.8 Hz, 2H). (M+H)+ 378.90.

### Example 24. Synthesis of 4-(4-ethyl-2-(4-fluorophenyl)-5-(1-methylpiperidin-4-yl)-1H-pyrrol-3-yl)pyridine

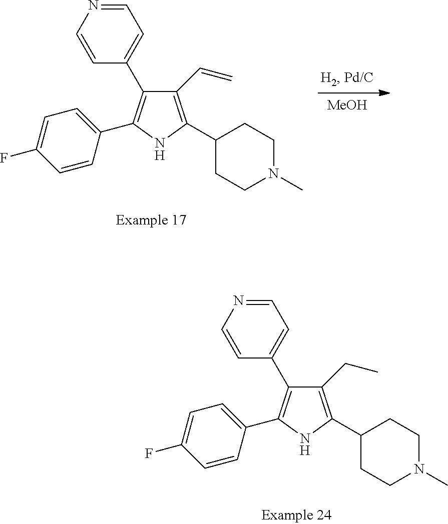 [SMILES: "MeOH" ; C=Cc1c(C2CCN(C)CC2)[nH]c(-c2ccc(F)cc2)c1-c1ccncc1 ; CCc1c(C2CCN(C)CC2)[nH]c(-c2ccc(F)cc2)c1-c1ccncc1]

Example 17 (44 mgs, 0.122 mmol, 1 eq) was dissolved in 6 mL MeOH. Palladium on carbon (30 mgs) was added to the methanolic solution. The reaction mixture was set up for hydrogenation using a hydrogen balloon. The reaction was stirred for 3 hours at room temperature and was determined complete by LC/MS analysis. The reaction mixture was filtered over celite and the filtrate was concentrated. Purification by silica gel chromatography (8% MeOH/CHCl₃ with 0.25% NH₃) afforded the title compound Example 24 4-(4-ethyl-2-(4-fluorophenyl)-5-(1-methylpiperidin-4-yl)-1H-pyrrol-3-yl)pyridine (15 mgs, 34% yield, 96% purity by LC/MS) as an off-white solid. ¹H NMR (400 MHz, DMSO-d₆) δ 10.72 (s, 1H), 8.48-8.39 (m, 2H), 7.20-7.00 (m, 6H), 2.89 (d, J=10.0 Hz, 2H), 2.66-2.52 (m, 1H), 2.35 (q, J=7.5 Hz, 2H), 2.22 (s, 3H), 2.09-1.88 (m, 4H), 1.62 (d, J=11.1 Hz, 2H), 0.86 (t, J=7.4 Hz, 3H). (M+H)+ 363.95.

### Example 25. Synthesis of 4-(2-(4-fluorophenyl)-5-(1-methylpiperidin-4-yl)-4-ethyl-1H-pyrrol-3-yl)pyridine

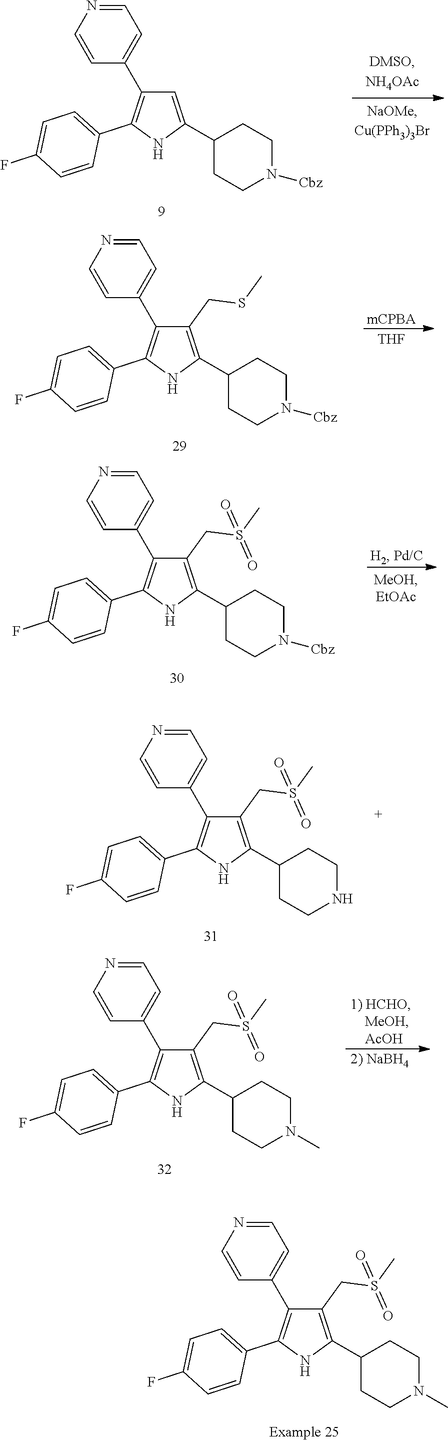

### a. Benzyl 4-(5-(4-fluorophenyl)-3-((methylthio)methyl)-4-(pyridin-4-yl)-1H-pyrrol-2-yl)piperidine-1-carboxylate (29)

Benzyl 4-(5-(4-fluorophenyl)-4-(pyridin-4-yl)-1H-pyrrol-2-yl)piperidine-1-carboxylate (9) (100 mgs, 0.22 mmol, 1 eq), Cu (PPh₃)₃Cl (31 mgs, 0.033 mmol, 0.15 eq), NH₄OAc (68 mgs, 0.88 mmol, 4 eq) and NaOMe 0.5M solution in methanol (0.44 mL, 0.22 mmol, 1 eq) were dissolved in DMSO (1.5 mL). The reaction was heated in the microwave for 48 hours at 140° C. after slow progression of heating the reaction in the hood. The reaction stalled after achieving 50% completion. 4 mL water was added to quench the reaction. The quenched mixture was partitioned between EtOAc and water and the aqueous phase was extracted with EtOAc (3×). The combined organic phase was dried over sodium sulphate and concentrated. Purification by silica gel chromatography (10%→25%→40% EtOAc/Hexanes) afforded benzyl 4-(5-(4-fluorophenyl)-3-((methylthio)methyl)-4-(pyridin-4-yl)-1H-pyrrol-2-yl)piperidine-1-carboxylate (29) (90 mgs, 80% yield, 97% purity by LC/MS). (M+H)+ 516.30.

### b. Benzyl 4-(5-(4-fluorophenyl)-3-((methylsulfonyl)methyl)-4-(pyridin-4-yl)-1H-pyrrol-2-yl)piperidine-1-carboxylate (30)

Benzyl 4-(5-(4-fluorophenyl)-3-((methylthio)methyl)-4-(pyridin-4-yl)-1H-pyrrol-2-yl)piperidine-1-carboxylate (29) (90 mgs, 0.175 mmol, 1 eq) was dissolved in 6 mL THF and cooled to 0° C. mCPBA (60 mgs, 0.349 mmol, 2 eq) was added portion wise and the reaction was stirred maintaining the temperature at 0° C. After 30 minutes, the reaction reached completion as determined by LC/MS analysis. 3 mL saturated NaHCO₃ was added to quench the reaction. The reaction mixture was partitioned between water and EtOAc and the aqueous phase was extracted with EtOAc (3×). The combined organic phase was dried over sodium sulphate and concentrated. Purification by silica gel chromatography (25%→50%→75%→100% EtOAc/Hexanes) gave benzyl 4-(5-(4-fluorophenyl)-3-((methylsulfonyl)methyl)-4-(pyridin-4-yl)-1H-pyrrol-2-yl)piperidine-1-carboxylate (30) (81 mgs, 85% yield, 99% purity by LC/MS). (M+H)+ 548.0.

### c. 4-(2-(4-fluorophenyl)-4-((methylsulfonyl)methyl)-5-(piperidin-4-yl)-1H-pyrrol-3-yl)pyridine (31) and 4-(2-(4-fluorophenyl)-5-(1-methylpiperidin-4-yl)-4-((methylsulfonyl)methyl)-1H-pyrrol-3-yl)pyridine (32)

Following the procedure as described for the synthesis of 21 and 22 using benzyl 4-(5-(4-fluorophenyl)-3-((methylsulfonyl)methyl)-4-(pyridin-4-yl)-1H-pyrrol-2-yl)piperidine-1-carboxylate (30) (75 mgs, 0.137 mmol, 1 eq) and palladium on carbon (50 mgs), the title compounds 4-(2-(4-fluorophenyl)-4-((methylsulfonyl)methyl)-5-(piperidin-4-yl)-1H-pyrrol-3-yl)pyridine (31) and 4-(2-(4-fluorophenyl)-5-(1-methylpiperidin-4-yl)-4-((methylsulfonyl)methyl)-1H-pyrrol-3-yl)pyridine (32) were isolated as a mixture (59 mgs). (M+H)+ 413.95, 427.85.

### d. 4-(2-(4-Fluorophenyl)-5(1-methylpiperidin-4-yl)-4-((methylsulfonyl)methyl)-1H-pyrrol-3-yl)pyridine

Following the procedure as described for the synthesis of Example 21, using a mixture of 4-(2-(4-fluorophenyl)-4-((methylsulfonyl)methyl)-5-(piperidin-4-yl)-1H-pyrrol-3-yl)pyridine (31) and 4-(2-(4-fluorophenyl)-5-(1-methylpiperidin-4-yl)-4-((methylsulfonyl)methyl)-1H-pyrrol-3-yl)pyridine (32) (59 mgs), HCHO 37% w/w solution in water (1.5 mL) and NaBH₄ (30 mgs), the title compound was synthesized. The crude product was dissolved in 1M HCl and partitioned between EtOAc and water. The organic phase was extracted with water (2×). The combined acidic aqueous phase was basified to pH 9 with 1N NaOH and EtOAc was added to this. The neutralised aqueous phase was extracted with EtOAc (3×). The combined organic phase was dried over sodium sulphate and concentrated to give purified 4-(2-(4-fluorophenyl)-5-(1-methylpiperidin-4-yl)-4-((methylsulfonyl)methyl)-1H-pyrrol-3-yl)pyridine (20 mgs, 98% purity by LC/MS) as an amorphous white solid. ¹H NMR (400 MHz, DMSO-d₆) δ 11.02 (s, 1H), 8.53-8.45 (m, 2H), 7.54-7.35 (m, 6H), 6.67 (s, 1H), 3.47 (d, =12.6 Hz, 2H), 3.35 (s, 3H), 3.07 (dt, J=13.4, 10.3 Hz, 2H), 2.96 (td, J=11.7, 5.8 Hz, 1H), 2.72 (d, J=4.5 Hz, 3H), 2.16-1.93 (m, 4H).

### Example 26. Synthesis of (1R,5S)-6-(5-(4-fluorophenyl)-4-(pyridin-4-yl)-1H-pyrrol-2-yl)-3-methyl-3-azabicyclo[3.1.0]hexane

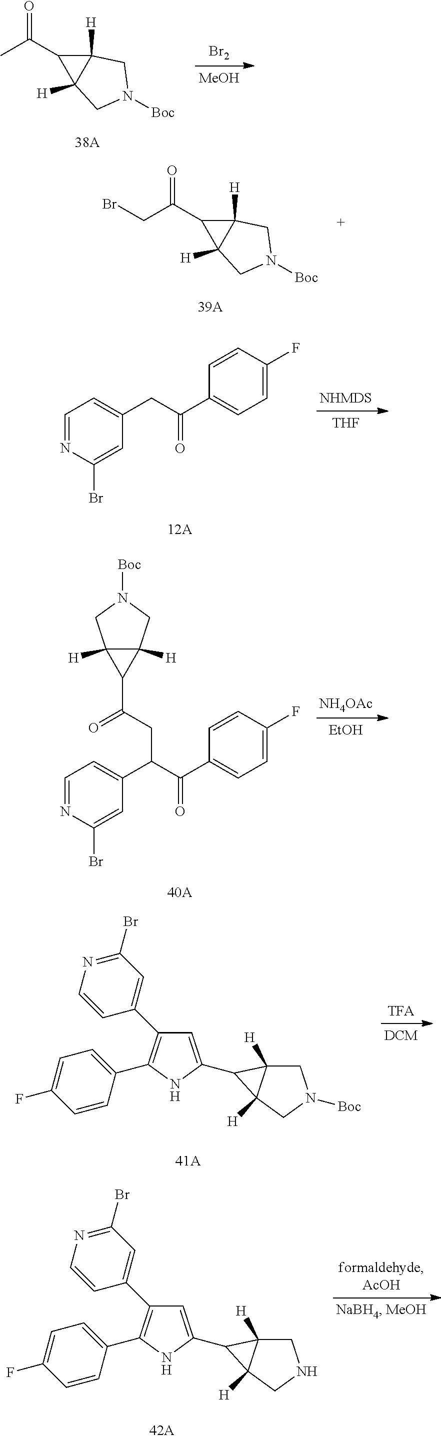

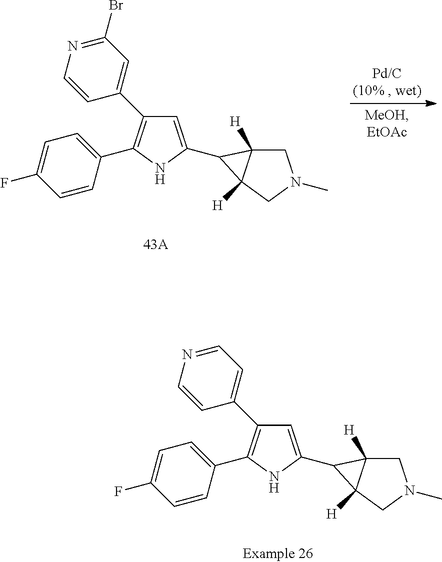

### a. tert-Butyl (1R,S)-6-(2-bromoacetyl)-3-azabicyclo[3.1.0]hexane-3-carboxylate (39A)

Following the procedure for the synthesis of (2A) using tert-butyl (1R,5S)-6-acetyl-3-azabicyclo[3.1.0]hexane-3-carboxylate (38A) (250 mg, 1.11 mmol, 1.0 eq), the compound (39A) (123 mg) was isolated as a crystalline white solid.

### b. tert-Butyl (1R,5S)-6-(3-(2-bromopyridin-4-yl)-4-(4-fluorophenyl)-4-oxobutanoyl)-3-azabicyclo[3.1.0]hexane-3-carboxylate (40A)

Following the procedure for the synthesis of (7A) using (12A) (118 mg, 0.40 mmol, 1.0 eq) and (39A) (121 mg, 0.40 mmol, 1.0 eq), the compound (40A) was isolated as a yellow oil and used as is in next reaction assuming quantitative yield. MS: 538.95, 541.00 [M+Na]⁺.

### c. tert-Butyl (1R,5S)-6-(4-(2-bromopyridin-4-yl)-5-(4-fluorophenyl)-1H-pyrrol-2-yl)-3-azabicyclo[3.1.0]hexane-3-carboxylate (41A)

Following the procedure for the synthesis of (8A) using (40A), the compound (41A) (152 mg, >99% purity by HPLC) was isolated as a glassy yellow solid. MS: 497.85, 499.05 [M+H]⁺.

### d. (1R,5S)-6-(4-(2-Bromopyridin-4-yl)-5-(4-fluorophenyl)-1H-pyrrol-2-yl)-3-azabicyclo[3.1.0]hexane (42A)

Following the procedure for the synthesis of (9A), using (41A) (147 mg, 0.294 mmol, 1.0 eq), the compound (42A), was isolated and used as is assuming quantitative yield. MS: 397.60, 399.05 [M+H]⁺.

### e. (1R,5S)-6-(4-(2-Bromopyridin-4-yl)-5-(4-fluorophenyl)-1H-pyrrol-2-yl)-3-methyl-3-azabicyclo[3.1.0]hexane (43A)

Following the procedure for the synthesis of (10A) using (42A) (117 mg, 0.294 mmol, 1.0 eq), the compound (43A) (118 mg, >98% purity by HPLC) was isolated as an off-white solid. MS: 411.80, 413.85 [M+H]⁺.

### f. (1R,5S)-6-(5-(4-Fluorophenyl)-4-(pyridin-4-yl)-1H-pyrrol-2-yl)-3-methyl-3-azabicyclo[3.1.0]hexane

To a solution of (43A) (60 mg, 0.146 mmol, 1.0 eq) in MeOH (5 mL) and EtOAc (5 mL) that was degassed under N₂, was added Pd/C (10%, wet, 15 mg). The reaction was (evacuated+backfilled with H₂)×4 and stirred overnight under H₂ at atmospheric pressure, room temperature. The reaction was determined complete by LC/MS and filtered over celite, washing with a mixture of MeOH and EtOAc. The filtrate concentrated under reduced pressure and purified by silica gel chromatography (0%→5%→10% MeOH/DCM with 1% 7N NH₃ in MeOH as a modifier) to yield the title compound Example 26 (34.3 mg, 100% purity by HPLC) as a pale yellow solid. MS: 334.00 [M+H]⁺. ¹H NMR (400 MHz, DMSO-d₆) δ 11.08 (s, 1H), 8.35-8.27 (m, 2H), 7.34-7.28 (m, 2H), 7.22-7.15 (m, 2H), 7.12-7.07 (m, 2H), 6.00 (d, J=2.6 Hz, 1H), 3.00 (d, J=8.8 Hz, 2H), 2.29 (d, J=9.2 Hz, 2H), 2.24 (s, 3H), 2.16 (t, J=3.3 Hz, 1H), 1.69 (ddd, J=3.2, 2.1, 1.1 Hz, 2H).

### Example 27. Synthesis of 3-(5-(4-fluorophenyl)-4-(pyridin-4-yl)-1H-pyrrol-2-yl)-8-methyl-8-azabicyclo[3.2.1]octane

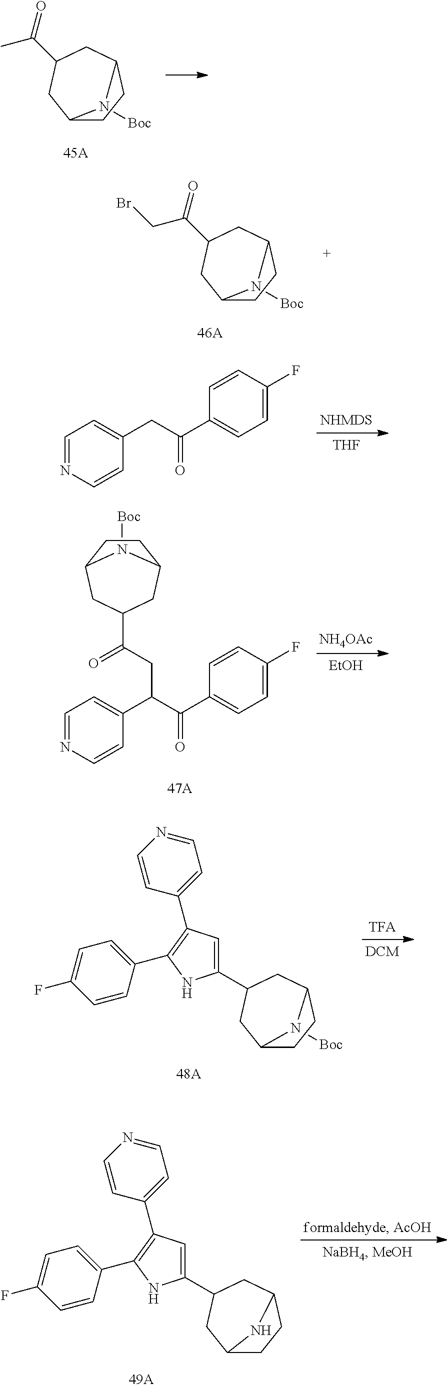

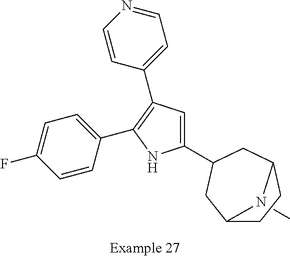 [SMILES: CN1C2CCC1CC(c1cc(-c3ccncc3)c(-c3ccc(F)cc3)[nH]1)C2]

### a. tert-Butyl 3-(2-bromoacetyl)-8-azabicyclo[3.2.1]octane-8-carboxylate (46A)

Following the procedure for the synthesis of (2A) tert-butyl 3-acetyl-8-azabicyclo[3.2.1]octane-8-carboxylate (45A) (265 mg, 1.05 mmol, 1.0 eq), the compound (46A) (185 mg, 100% purity by HPLC) was isolated as a crystalline white solid.

### b. tert-Butyl 3-(4-(4-fluorophenyl)-4-oxo-3-(pyridin-4-yl)butanoyl)-8-azabicyclo[3.2.1]octane-8-carboxylate (47A)

Following the procedure for the synthesis of (7A) using 1-(4-fluorophenyl)-2-(pyridin-4-yl)ethan-1-one (117 mg, 0.542 mmol, 1.0 eq) and (39A) (180 mg, 0.542 mmol, 1.0 eq), the compound (47A) was isolated as a yellow oil and used as is in next reaction assuming quantitative yield. MS: 467.05 [M+H]⁺.

### c. tert-Butyl 3-(5-(4-fluorophenyl)-4-(pyridin-4-yl)-1H-pyrrol-2-yl)-8-azabicyclo[3.2.1]octane-8-carboxylate (48A)

Following the procedure for the synthesis of (8A) using (47A), the compound (48A) (78 mg, >99% purity by HPLC) was isolated as a glassy yellow solid. MS: 448.05 [M+H]⁺.

### d. 3-(5-(4-Fluorophenyl)-4-(pyridin-4-yl)-1H-pyrrol-2-yl)-8-azabicyclo[3.2.1]octane (49A)

Following the procedure for the synthesis of (9A), using (48A) (76 mg, 0.170 mmol, 1.0 eq), the compound (49A) (95% purity) was isolated and used as is assuming quantitative yield. MS: 347.90 [M+H]⁺.

### e. 3-(5-(4-fluorophenyl)-4-(pyridin-4-yl)-1H-pyrrol-2-yl)-8-methyl-8-azabicyclo[3.2.1]octane

Following the procedure for the synthesis of Example 1, using (49A) (59 mg, 0.170 mmol, 1.0 eq), the title compound Example 27 (33.0 mg, >99% purity by HPLC) was isolated as a pale yellow solid. MS: 361.85 [M+H]⁺. ¹H NMR (400 MHz, DMSO-d₆) δ 11.04 (s, 1H), 8.31 (d, J=6.2 Hz, 2H), 7.38-7.25 (m, 2H), 7.25-7.14 (m, 2H), 7.14-7.05 (m, 2H), 6.08 (d, J=2.5 Hz, 1H), 3.09 (s, 2H), 2.87 (dt, J=12.3, 6.5 Hz, 1H), 2.17 (s, 3H), 1.97 (dd, J=9.2, 4.1 Hz, 2H), 1.76 (dd, J=13.5, 11.0 Hz, 2H), 1.65 (dt, J=12.6, 3.9 Hz, 2H), 1.58 (q, J=6.2 Hz, 2H).

### Example 28. Synthesis of 4-(5-(1,4-dimethylpiperidin-4-yl)-2-(4-fluorophenyl)-1H-pyrrol-3-yl)pyridine

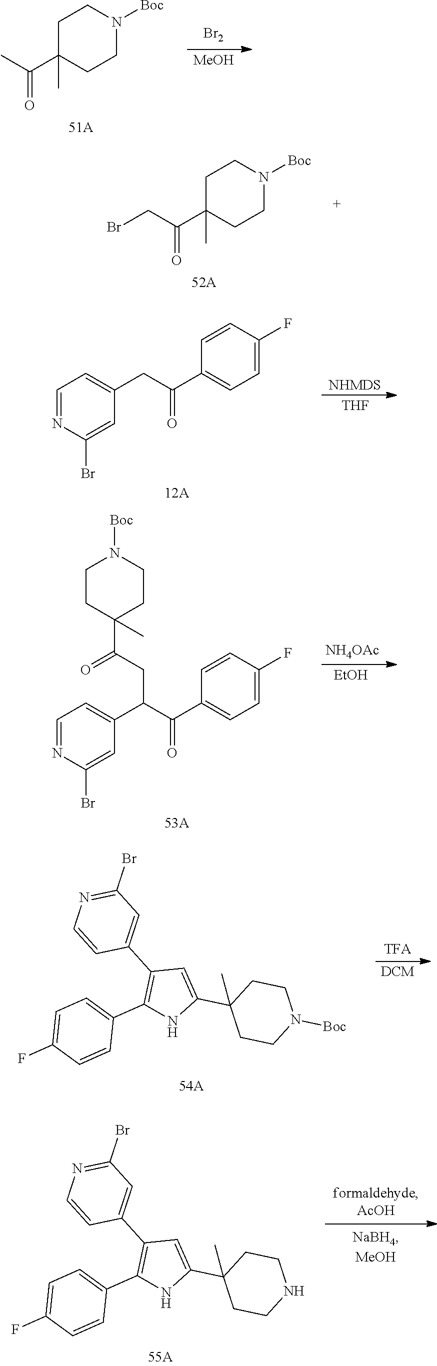

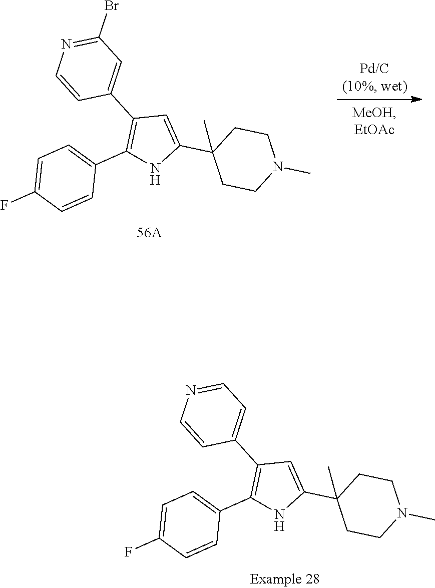 [SMILES: CN1CCC(C)(c2cc(-c3ccnc(Br)c3)c(-c3ccc(F)cc3)[nH]2)CC1 ; CN1CCC(C)(c2cc(-c3ccncc3)c(-c3ccc(F)cc3)[nH]2)CC1]

### a. tert-Butyl 4-(2-bromoacetyl)-4-methylpiperidine-1-carboxylate (52A)

Following the procedure for the synthesis of (2A) using tert-butyl 4-acetyl-4-methylpiperidine-1-carboxylate (51A) (333 mg, 1.38 mmol, 1.0 eq), the compound (52A) (286 mg) was isolated as a pale yellow oil.

### b. tert-Butyl 4-(3-(2-bromopyridin-4-yl)-4-(4-fluorophenyl)-4-oxobutanoyl)-4-methylpiperidine-1-carboxylate (53A)

Following the procedure for the synthesis of (7A) using (12A) (258 mg, 0.880 mmol, 1.0 eq) and (52A) (280 mg, 0.880 mmol, 1.0 eq), the compound (53A) was isolated as a yellow oil and used as is in next reaction assuming quantitative yield. MS: 555.10, 557.05 [M+Na]⁺.

### c. tert-Butyl 4-(4-(2-bromopyridin-4-yl)-5-(4-fluorophenyl)-1H-pyrrol-2-yl)-4-methylpiperidine-1-carboxylate (54A)

Following the procedure for the synthesis of (8A) using (53A), the compound (54A) (143 mg, 90% purity by HPLC) was isolated as a light yellow solid. MS: 513.70, 515.90 [M+H]⁺.

### d. 2-Bromo-4-(2-(4-fluorophenyl)-5-(4-methylpiperidin-4-yl)-1H-pyrrol-3-yl)pyridine (55A)

Following the procedure for the synthesis of (9A), using (54A) (140 mg, 0.272 mmol, 1.0 eq), the compound (55A) (89% purity by HPLC) was used as is assuming quantitative yield. MS: 413.75, 415.85 [M+H]⁺.

### e. 2-Bromo-4-(5-(1,4-dimethylpiperidin-4-yl)-2-(4-fluorophenyl)-1H-pyrrol-3-yl)pyridine (56A)

Following the procedure for the synthesis of (10A) using (55A) (113 mg, 0.272 mmol, 1.0 eq), the compound (56A) (101.2 mg, >99% purity by HPLC) was isolated as a light yellow solid. MS: 427.80, 429.85 [M+H]⁺.

### f. 4-(5-(1,4-Dimethylpiperidin-4-yl)-2-(4-fluorophenyl)-1H-pyrrol-3-yl)pyridine

Following the procedure for the synthesis of (44A) using (56A) (100 mg, 0.233 mmol, 1 eq), the title compound Example 28 (90.5 mg, 100% purity by HPLC) was isolated as a light yellow solid. MS: 349.85 [M+H]⁺. ¹H NMR (400 MHz, DMSO-d₆) δ 11.08 (d, J=2.5 Hz, 1H), 8.32 (d, J=4.8 Hz, 2H), 7.37-7.29 (m, 2H), 7.23-7.16 (m, 2H), 7.16-7.11 (m, 2H), 6.10 (d, J=2.5 Hz, 1H), 2.88 (s, 1H), 2.59 (d, J=12.8 Hz, 1H), 2.21 (s, 3H), 2.15-1.80 (m, 4H), 1.62 (qd, J=12.7, 3.5 Hz, 1H), 1.36 (q, J=12.1 Hz, 1H), 1.05 (d, J=6.1 Hz, 3H).

### Examples 29 and 30. Synthesis of 4-(5-(cis-1,2-dimethylpiperidin-4-yl)-2-(4-fluorophenyl)-1H-pyrrol-3-yl)pyridine and 4-(5-(trans-1,2-dimethylpiperidin-4-yl)-2-(4-fluorophenyl)-1H-pyrrol-3-yl)pyridine

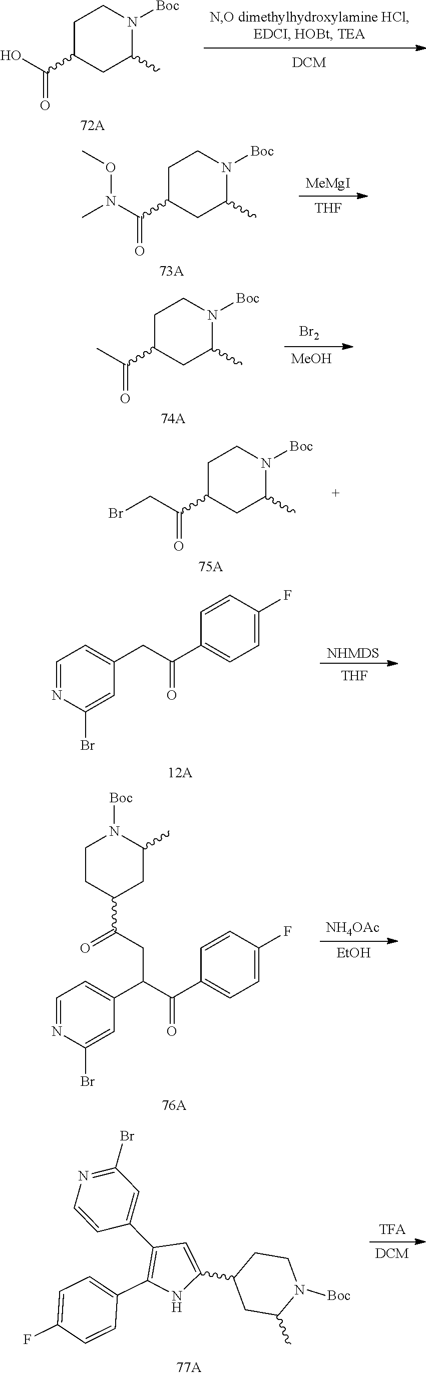

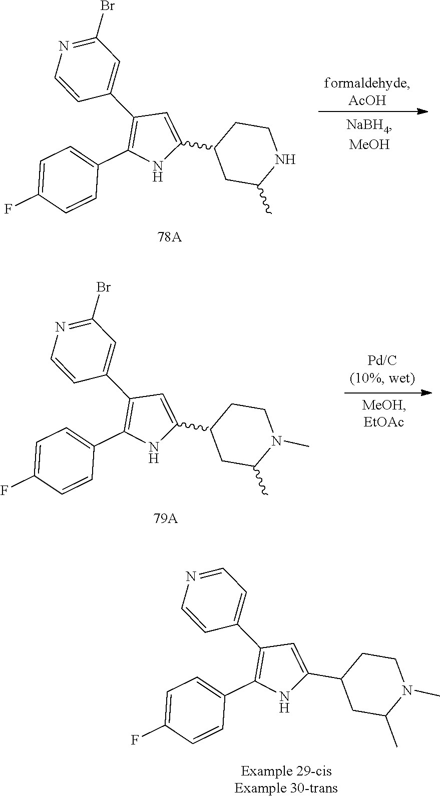

### a. tert-Butyl 4-(methoxy(methyl)carbamoyl)-2-methylpiperidine-1-carboxylate (73A)

Following the procedure for the synthesis of (60A) using (72A) (500 mg, 2.06 mmol, 1.0 eq), the compound (73A) (526 mg, 1.84 mmol) was isolated as a clear oil.

### b. tert-Butyl 4-acetyl-2-methylpiperidine-1-carboxylate (74A)

Following the procedure for the synthesis of (61A) using (73A) (520 mg, 1.82 mmol, 1.0 eq), the compound (74A) (355 mg, 1.47 mmol) was isolated as a pale orange oil.

### c. tert-Butyl 4-(2-bromoacetyl)-2-methylpiperidine-1-carboxylate (75A)

Following the procedure for the synthesis of (62A) using (74A) (345 mg, 1.43 mmol, 1.0 eq), the compound (75A) (335 mg, 1.05 mmol) was isolated as a light brown oil and used without further purification.

### d. tert-Butyl 4-(3-(2-bromopyridin-4-yl)-4-(4-fluorophenyl)-4-oxobutanoyl)-2-methylpiperidine-1-carboxylate (76A)

Following the procedure for the synthesis of (7A) using (12A) (330 mg, 1.03 mmol, 1.0 eq) and (75A) (303 mg, 1.03 mmol, 1.0 eq), the compound (76A) was isolated as a yellow oil and used as is in next reaction assuming quantitative yield. MS: 554.95, 556.95 [M+Na]⁺.

### e. tert-Butyl 4-(4-(2-bromopyridin-4-yl)-5-(4-fluorophenyl)-1H-pyrrol-2-yl)-2-methylpiperidine-1-carboxylate (77A)

Following the procedure for the synthesis of (8A) using (76A), the compound (77A) (118 mg, 0.23 mmol, 95% purity by HPLC) was isolated as a glassy yellow solid and a mixture of isomers. MS: 513.80, 515.90 [M+H]⁺.

### f. 2-Bromo-4-(2-(4-fluorophenyl)-5-(2-methylpiperidin-4-yl)-1H-pyrrol-3-yl)pyridine (78A)

Following the procedure for the synthesis of (9A) using (77A) (115 mg, 0.23 mmol, 1.0 eq), the compound (78A) (95% purity by HPLC) was isolated as a yellow solid and used as is in the next reaction assuming quantitative yield. MS: 413.70, 415.70 [M+H]⁺.

### g. 2-Bromo-4-(5-(1,2-dimethylpiperidin-4-yl)-2(4-fluorophenyl)-1H-pyrrol-3-yl)pyridine (79A)

Following the procedure for the synthesis of (10A) using (78A) (95 mg, 0.23 mmol, 1.0 eq), the compound (79A) (96 mg, 0.23 mmol, 95% purity by HPLC) was isolated as a yellow solid and used as is in the next reaction assuming quantitative yield. MS: 427.80, 429.75 [M+H]⁺.

### h. 4-(5-(cis-1,2-dimethylpiperidin-4-yl)-2-(4-fluorophenyl)-1H-pyrrol-3-yl)pyridine and 4-(5-(trans-1,2-dimethylpiperidin-4-yl)-2-(4-fluorophenyl)-1H-pyrrol-3-yl)pyridine

Following the procedure for the synthesis of (44A) using (79A) (93 mg, 0.217 mmol, 1.0 eq), the title compounds (cis stereochemistry, diastereomer 1, Example 29) (19.3 mg, 0.055 mmol, 100% purity by HPLC) and (trans stereochemistry, diastereomer 2, Example 30) (27.1 mg, 0.078 mmol, 100% purity by HPLC) were isolated as light yellow solids.

Example 29: MS: 349.85 [M+H]⁺. ¹H NMR (400 MHz, DMSO-d₆) δ 11.08 (d, J=2.5 Hz, 1H), 8.32 (d, J=4.8 Hz, 2H), 7.37-7.29 (m, 2H), 7.23-7.16 (m, 2H), 7.16-7.11 (m, 2H), 6.10 (d, J=2.5 Hz, 1H), 2.88 (s, 1H), 2.59 (d, J=12.8 Hz, 1H), 2.21 (s, 3H), 2.15-1.80 (m, 4H), 1.62 (qd, J=12.7, 3.5 Hz, 1H), 1.36 (q, J=12.1 Hz, 1H), 1.05 (d, J=6.1 Hz, 3H).

Example 30: MS: 349.65 [M+H]⁺. 1H NMR (400 MHz, DMSO-d₆) δ 11.04 (s, 1H), 8.38-8.27 (m, 2H), 7.37-7.30 (m, 2H), 7.23-7.16 (m, 2H), 7.16-7.12 (m, 2H), 6.16 (d, J=2.4 Hz, 1H), 2.90 (m, 1H), 2.78 (s, 1H), 2.60 (s, 1H), 2.46-2.40 (m, 1H), 2.24 (s, 3H), 2.00-1.66 (m, 4H), 1.02 (d, J=6.5 Hz, 3H).

### Examples 31 and 32. Synthesis of 4-(5-(cis-1,3-dimethylpiperidin-4-yl)-2-(4-fluorophenyl)-1H-pyrrol-3-yl)pyridine (diastereomer 1) and 4-(5-(trans-1,3-dimethylpiperidin-4-yl)-2-(4-fluorophenyl)-1H-pyrrol-3-yl)pyridine (diastereomer 2)

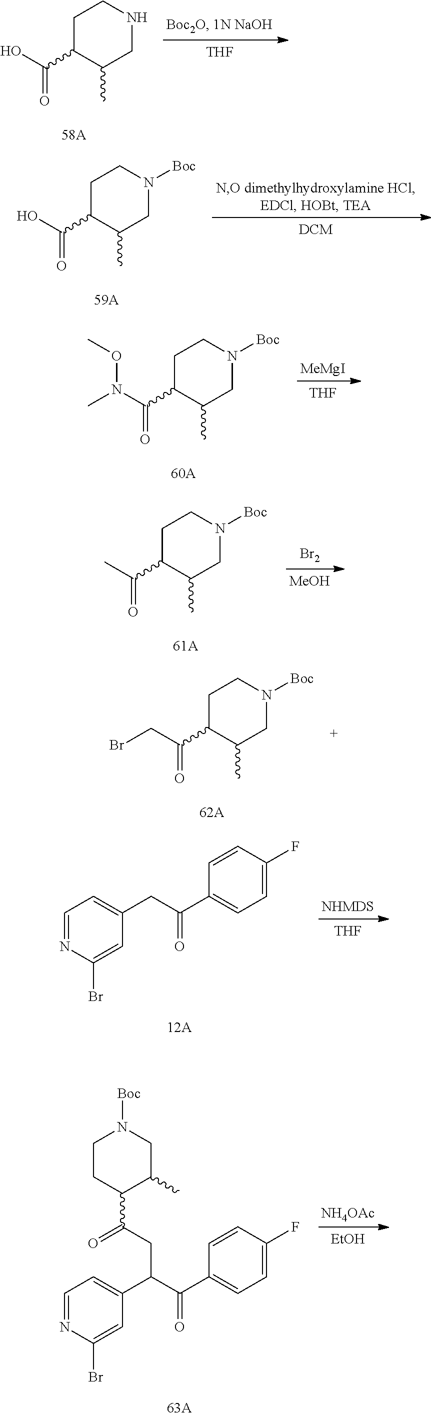

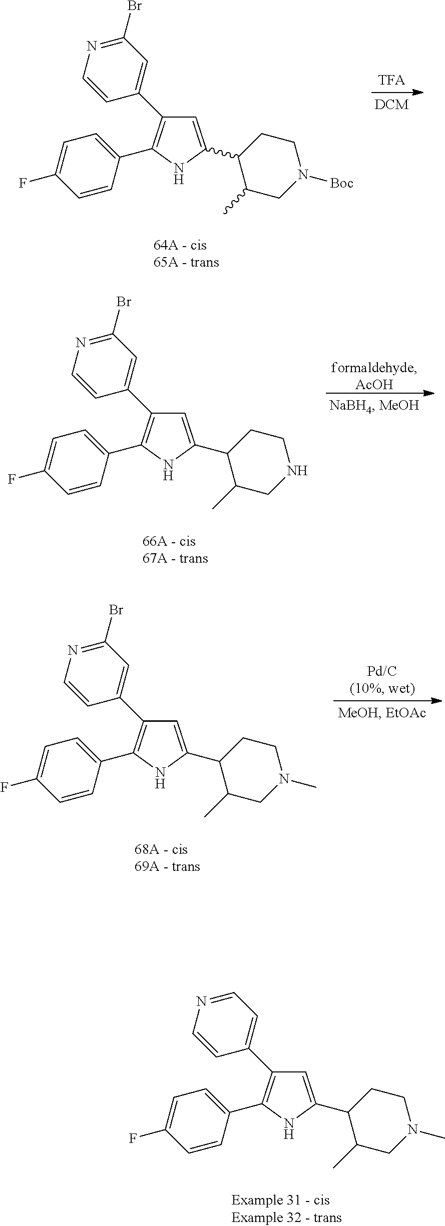

### a. 1-(tert-Butoxycarbonyl)-3-methylpiperidine-4-carboxylic acid (59A)

To a solution of 3-methylpiperidine-4-carboxylic acid (58A) (250 mg, 1.75 mmol, 1.0 eq) in THF (5 mL) and 1M NaOH(aq) (5 mL) was added boc anhydride (764 mg, 3.5 mmol, 2.0 eq). After stirring overnight at room temperature the reaction pH was adjusted to 5 with 1M HCl (aq), and was partitioned in EtOAc/water. The organic was separated and the aqueous was extracted 1×EtOAc. The combined organic was dried over Na₂SO₄, filtered and concentrated to afford the compound (59A) (363 mg, 1.49 mmol) as a white crystalline solid.

### b. tert-Butyl 4-(methoxy(methyl)carbamoyl)-3-methylpiperidine-1-carboxylate (60A)

To a mixture of 1-(tert-butoxycarbonyl)-3-methylpiperidine-4-carboxylic acid (59A) (350 mg, 1.44 mmol, 1.0 eq), EDCI (414 mg, 2.16 mmol, 1.5 eq), HOBt (292 mg, 2.16 mmol, 1.5 eq), and N,O dimethylhydroxylamine HCl (211 mg, 2.16 mmol, 1.5 eq) in DCM (15 mL) was added TEA (602 μl, 4.32 mmol, 3.0 eq). The reaction was stirred overnight at room temperature and concentrated under reduced pressure. The residue was partitioned in EtOAc/water. The organic was separated and washed 1× water, 1× brine, dried over Na₂SO₄, filtered and concentrated. Purification by silica gel chromatography (10%→50% EtOAc/Hex) afforded the compound (60A) (328 mg, 1.15 mmol) as a clear oil after concentration of product fractions.

### c. tert-Butyl 4-acetyl-3-methylpiperidine-1-carboxylate (61A)

To a solution of tert-butyl 4-(methoxy(methyl)carbamoyl)-3-methylpiperidine-1-carboxylate (60A) (320 mg, 1.12 mmol, 1.0 eq) in THF (6 mL) was added MeMgI (3M in Et₂O, 747 μl, 2.24 mmol, 2.0 eq). After stirring overnight at room temperature, the reaction was determined complete by TLC (25% EtOAc/Hex) after quenching an aliquot with NH₄Cl (sat. aq) and staining with PMA solution. The remainder of the reaction was quenched with NH₄Cl (sat, aq) and stirred for 1 hour at room temperature. The quenched reaction was partitioned in EtOAc/water, the organic was separated, the aqueous was extracted 1×EtOAc, the combined organic was dried over Na₂SO₄, filtered and concentrated. The residue was purified by silica gel chromatography (10%→25% EtOAc/Hex) to afford the compound (61A) (232 mg, 0.96 mmol) as a clear oil after concentration of product fractions.

### d. tert-Butyl 4-(2-bromoacetyl)-3-methylpiperidine-1-carboxylate (62A)

To a solution of tert-butyl 4-acetyl-3-methylpiperidine-1-carboxylate (61A) (225 mg, 0.93 mmol, 1.0 eq) in methanol (5 mL) was added dropwise bromine (149 mg, 0.93 mmol, 1 eq). The reaction went from orange to clear over the course of 2 hours, at which point the reaction was monitored by TLC (25% EtOAc/Hex) and stained with PMA solution to show complete conversion. The crude reaction was partitioned in EtOAc/50% brine. The organic was separated and the aqueous was extracted 2×EtOAc. The combined organics were dried over Na₂SO₄, filtered and concentrated to yield the compound (62A) (254 mg, 0.79 mmol) as a clear oil after concentration, that was used without further purification.

### e. tert-Butyl 4-(3-(2-bromopyridin-4-yl)-4-(4-fluorophenyl)-4-oxobutanoyl)-3-methylpiperidine-1-carboxylate (63A)

Following the procedure for the synthesis of (7A) using (12A) (232 mg, 0.79 mmol, 1.0 eq) and (62A) (254 mg, 0.79 mmol, 1.0 eq), the compound (63A) was isolated as a yellow oil and used as is in next reaction assuming quantitative yield. MS: 554.75, 556.85 [M+Na]⁺.

### f. tert-Butyl cis-4-(4-(2-bromopyridin-4-yl)-5-(4-fluorophenyl)-1H-pyrrol-2-yl)-3-methylpiperidine-1-carboxylate (64A) and tert-butyl trans-4-(4-(2-bromopyridin-4-yl)-5-(4-fluorophenyl)-1H-pyrrol-2-yl)-3-methylpiperidine-1-carboxylate (65A)

Following the procedure for the synthesis of (8A) using (63A), the compounds (64A) (73.1 mg, 0.14 mmol, 85% purity by HPLC) and (65A) (128 mg, 0.25 mmol, 96% purity by HPLC) were isolated as yellow solids after careful separation by silica gel chromatography (5%→10%→25% EtOAc/Hex). (64A) MS: 514.00, 516.05 [M+H]⁺. (65A) MS: 513.70, 516.00 [M+H]⁺.

### g. 2-Bromo-4-(2-(4-fluorophenyl)-5-(cis-3-methylpiperidin-4-yl)-1H-pyrrol-3-yl)pyridine (66A)

Following the procedure for the synthesis of (9A) using (64A) (72 mg, 0.14 mmol, 1.0 eq), the compound (66A) was isolated as a yellow solid and used as is in the next reaction assuming quantitative yield. MS: 413.60, 416.45 [M+H]⁺.

### h. 2-Bromo-4-(2-(4-fluorophenyl)-5-(trans-3-methylpiperidin-4-yl)-1H-pyrrol-3-yl)pyridine (67A)

Following the procedure for the synthesis of (9A) using (65A) (72 mg, 0.14 mmol, 1.0 eq), the compound (67A) was isolated as a yellow solid and used as is in the next reaction assuming quantitative yield. MS: 413.80, 415.75 [M+H]⁺.

### i. 2-Bromo-4-(5-(cis-1,3-dimethylpiperidin-4-yl)-2-(4-fluorophenyl)-1H-pyrrol-3-yl)pyridine (68A)

Following the procedure for the synthesis of (10A) using (66A) (58 mg, 0.14 mmol, 1.0 eq), the compound (68A) (62.5 mg, quantitative yield, 90% purity by HPLC) was isolated as a glassy yellow solid and used as is without further purification. MS: 427.65, 429.70 [M+H]⁺.

### j. 2-Bromo-4-(5-(trans-1,3-dimethylpiperidin-4-yl)-2-(4-fluorophenyl)-1H-pyrrol-3-yl)pyridine (69A)

Following the procedure for the synthesis of (10A) using (67A) (101.5 mg, 0.245 mmol, 1.0 eq), the compound (69A) (112 mg, quantitative yield, 100% purity by HPLC) was isolated as a light yellow solid and used as is without further purification. MS: 427.40, 429.55 [M+H]⁺.

### k. 4-(5-(cis-1,3-Dimethylpiperidin-4-yl)-2-(4-fluorophenyl)-1H-pyrrol-3-yl)pyridine (diastereomer 1, Example 31)

Following the procedure for the synthesis of (44A) using (68A) (60 mg, 0.14 mmol, 1.0 eq), the compound with cis stereochemistry (29.5 mg, 0.084 mmol, 100% purity by HPLC) was isolated as a light yellow solid. MS: 350.35 [M+H]⁺. ¹H NMR (400 MHz, DMSO-d₆) δ 11.19 (s, 1H), 8.40-8.29 (m, 2H), 7.38-7.30 (m, 2H), 7.25-7.17 (m, 2H), 7.17-7.13 (m, 2H), 6.17 (d, J=2.6 Hz, 1H), 3.23 (m, 3H), 2.60 (s, 3H), 2.33 (m, 2H), 2.06-1.79 (m, 3H), 0.81 (d, J=6.5 Hz, 3H).

### l. 4-(5-(trans-1,3-Dimethylpiperidin-4-yl)-2-(4-fluorophenyl)-1H-pyrrol-3-yl)pyridine (diastereomer 2, Example 32)

Following the procedure for the synthesis of (44A) using (69A) (105 mg, 0.245 mmol, 1.0 eq), the title compound with trans stereochemistry the title compound Example 32 (66.7 mg, 0.191 mmol, 100% purity by HPLC) was isolated as a light yellow solid. MS: 350.10 [M+H]⁺. ¹H NMR (400 MHz, DMSO-d₆) δ 11.11 (s, 1H), 8.40-8.28 (m, 2H), 7.38-7.29 (m, 2H), 7.26-7.12 (m, 4H), 6.18 (s, 1H), 3.21-2.69 (m, 4H), 2.65 (m, J=1.9 Hz, 1H), 2.31 (m, J=1.9 Hz, 3H), 1.98 (m, 3H), 0.83 (d, J=7.1 Hz, 3H).

### Example 33. Synthesis of 4-(5-(1-methylpiperidin-4-yl)-2-(4-(trifluoromethyl)phenyl)-1H-pyrrol-3-yl)pyridine

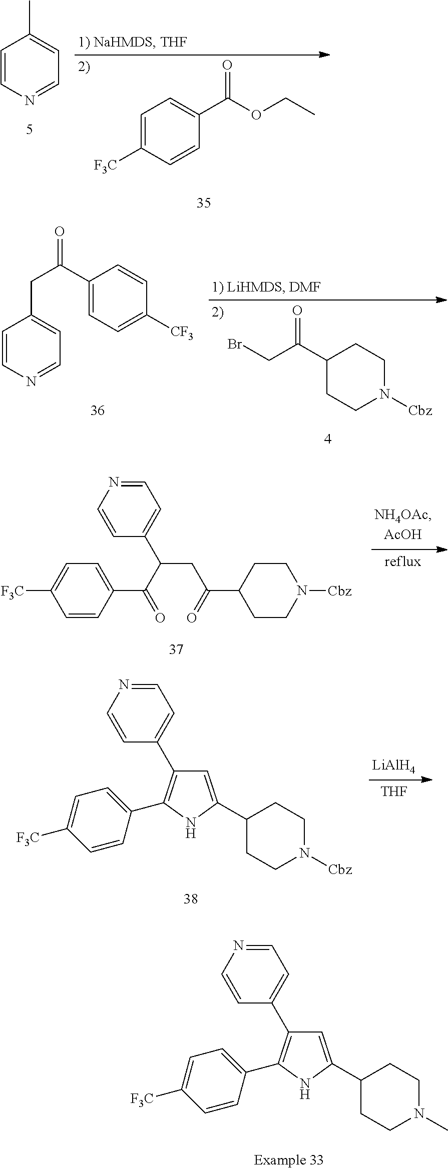

### a. 2-(Pyridin-4-yl)-1-(4-(trifluoromethyl)phenyl)ethan-1-one (36)

Following the procedure as described for the synthesis of 1-(4-fluorophenyl)-2-(pyridin-4-yl)ethan-1-one (7) using 4-methylpyridine (5) (196 mgs, 2.1 mmol, 1 eq), NaHMDS 1M solution in THF (3.15 mL, 3.15 mmol, 1.5 eq) and Ethyl 4-trifluoromethyl benzoate (35) (504 mgs, 2.31 mmol, 1.1 eq), the title compound (36) was synthesized. Purification using silica gel chromatography (10%→25%→50% EtOAc/Hexanes) afforded 2-(pyridin-4-yl)-1-(4-(trifluoromethyl)phenyl)ethan-1-one (36) (225 mgs, 40% yield 88% purity by LC/MS). (M+H)+ 265.80.

### b. Benzyl 4-(4-oxo-3-(pyridin-4-yl)-4-(4-(trifluoromethyl)phenyl)butanoyl)piperidine-1-carboxylate (37)

Following the procedure as described for the synthesis of benzyl 4-(4-(4-fluorophenyl)-4-oxo-3-(pyridin-4-yl)butanoyl)piperidine-1-carboxylate (8) using 2-(pyridin-4-yl)-1-(4-(trifluoromethyl)phenyl)ethan-1-one (36) (213 mgs, 0.803 mmol, 1 eq), LiHMDS 1M solution in THF (1 mL, 1 mmol, 1.25 mmol) and benzyl 4-(2-bromoacetyl)piperidine-1-carboxylate (4) (382 mgs, 1.124 mmol, 1.4 eq), the compound benzyl 4-(4-oxo-3-(pyridin-4-yl)-4-(4-(trifluoromethyl)phenyl)butanoyl)piperidine-1-carboxylate (37) (690 mgs) was synthesized which was taken onto the next step without any purification. (M+H)+ 525.05.

### c. Benzyl 4-(4-(pyridin-4-yl)-5-(4-(trifluoromethyl)phenyl)-1H-pyrrol-2-yl)piperidine-1-carboxylate (38)

Following the procedure as described for the synthesis of benzyl 4-(5-(4-fluorophenyl)-4-(pyridin-4-yl)-1H-pyrrol-2-yl)piperidine-1-carboxylate (9) using crude benzyl 4-(4-oxo-3-(pyridin-4-yl)-4-(4-(trifluoromethyl)phenyl)butanoyl)piperidine-1-carboxylate (37) (690 mgs) and NH₄OAc (2 g), the title compound (38) was synthesized. Purification by silica gel chromatography (10%→25%→50% EtOAc/Hexanes) afforded benzyl 4-(4-(pyridin-4-yl)-5-(4-(trifluoromethyl)phenyl)-1H-pyrrol-2-yl)piperidine-1-carboxylate (38) (115 mgs, 75% purity by LC/MS). (M+H)+ 506.0.

### d. 4-(5-(1-Methylpiperidin-4-yl)-2-(4-(trifluoromethyl)phenyl)-1H-pyrrol-3-yl)pyridine

Following the procedure as described for the synthesis of 4-(2-(4-fluorophenyl)-5-(1-methylpiperidin-4-yl)-1H-pyrrol-3-yl)pyridine (10) using benzyl 4-(4-(pyridin-4-yl)-5-(4-(trifluoromethyl)phenyl)-1H-pyrrol-2-yl)piperidine-1-carboxylate (38) (102 mgs, 0.202 mmol, 1 eq) and LAH 2M solution in THF (0.4 mL, 0.8 mmol, 4 eq), the title compound was synthesized. The crude product was dissolved in 1N HCl and partitioned between EtOAc and water. The organic phase was extracted with water (2×). The combined acidic aqueous phase was basified to pH 9 with 1N NaOH and EtOAc was added. The neutralised aqueous phase was extracted with EtOAc (3×). The combined organic phase was dried over sodium sulphate and concentrated. Purification by silica gel chromatography (2%→5%→8%→10% MeOH/CHCl₃ with 0.25% NH₃) afforded the title compound Example 33 4-(5-(1-methylpiperidin-4-yl)-2-(4-(trifluoromethyl)phenyl)-1H-pyrrol-3-yl)pyridine (30 mgs, 38% yield, >99% purity by LC/MS) as a pale-yellow solid. ¹H NMR (500 MHz, DMSO-d₆) δ 11.32 (d, =2.6 Hz, 1H), 8.41-8.36 (m, 2H), 7.68 (d, J=8.3 Hz, 2H), 7.54-7.48 (m, 2H), 7.22-7.15 (m, 2H), 6.14 (d, J=2.4 Hz, 1H), 2.82 (dt, J=12.0, 3.1 Hz, 2H), 2.16 (s, 3H), 1.99-1.85 (m, 4H), 1.72-1.55 (m, 3H). (M+H)+ 386.05.

### Example 34. Synthesis of 4-(2-(4-methoxyphenyl)-5-(1-methylpiperidin-4-yl)-1H-pyrrol-3-yl)pyridine

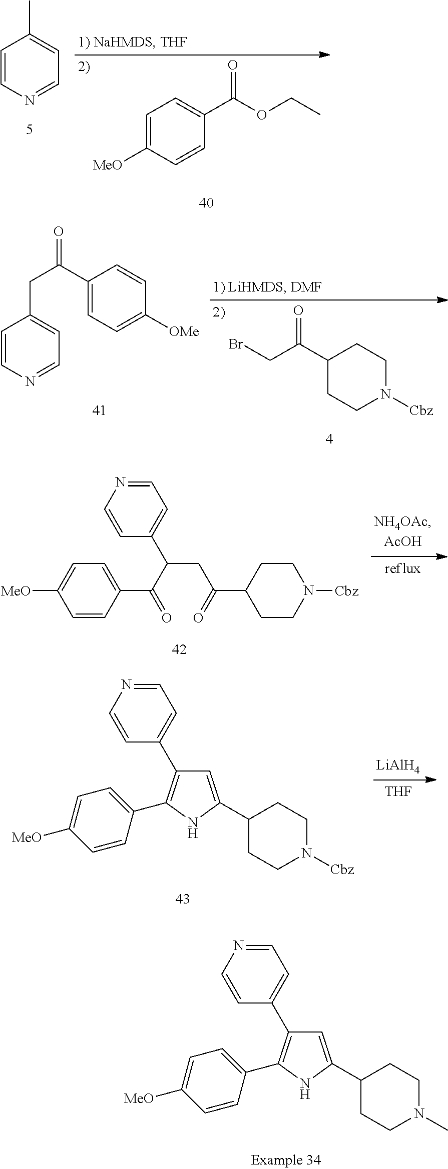

### a. 1-(4-Methoxyphenyl)-2-(pyridin-4-yl)ethan-1-one (41)

Following the procedure as described for the synthesis of 1-(4-fluorophenyl)-2-(pyridin-4-yl)ethan-1-one (7) using 4-methylpyridine (5) (464 mgs, 4.98 mmol, 1 eq), NaHMDS 1M solution in THF (7.4 mL, 7.4 mmol, 1.5 eq) and methyl 4-methoxybenzoate (40) (1.08 g, 6.5 mmol, 1.3 eq), the title compound (41) was synthesized. Purification by silica gel chromatography (10%→25%→50% EtOAc/Hexanes) afforded 1-(4-methoxyphenyl)-2-(pyridin-4-yl)ethan-1-one (41) (229 mgs, 20% yield, 98% purity by LC/MS). (M+H)+ 227.75.

### b. Benzyl 4-(4-(4-methoxyphenyl)-4-oxo-3-(pyridin-4-yl)butanoyl)piperidine-1-carboxylate (42)

Following the procedure as described for the synthesis of benzyl 4-(4-(4-fluorophenyl)-4-oxo-3-(pyridin-4-yl)butanoyl)piperidine-1-carboxylate (8) using 1-(4-methoxyphenyl)-2-(pyridin-4-yl)ethan-1-one (41) (215 mgs, 0.947 mmol, 1 eq), LiHMDS 1M solution in THF (1.15 mL, 1.15 mmol, 1.2 eq) and benzyl 4-(2-bromoacetyl)piperidine-1-carboxylate (4) (415 mgs, 1.231 mmol, 1.3 eq), the title compound benzyl 4-(4-(4-methoxyphenyl)-4-oxo-3-(pyridin-4-yl)butanoyl)piperidine-1-carboxylate (42) (580 mgs) was synthesized which was taken onto the next step without any purification. (M+H)+ 486.95.

### c. Benzyl 4-(5-(4-methoxyphenyl)-4-(pyridin-4-yl)-1H-pyrrol-2-yl)piperidine-1-carboxylate (43)

Following the procedure as described for the synthesis of benzyl 4-(5-(4-fluorophenyl)-4-(pyridin-4-yl)-1H-pyrrol-2-yl)piperidine-1-carboxylate (9) using crude benzyl 4-(5-(4-methoxyphenyl)-4-(pyridin-4-yl)-1H-pyrrol-2-yl)piperidine-1-carboxylate (43) (580 mgs) and NH₄OAc (2 g), the title compound (43) was synthesized. Purification by silica gel chromatography (10%→25%→50% EtOAc/Hexanes) afforded benzyl 4-(5-(4-methoxyphenyl)-4-(pyridin-4-yl)-1H-pyrrol-2-yl)piperidine-1-carboxylate (43) (288 mgs, 89% purity by LC/MS). (M+H)+ 468.15.

### d. 4-(2-(4-methoxyphenyl)-5-(1-methylpiperidin-4-yl)-1H-pyrrol-3-yl)pyridine

Following the procedure as described for the synthesis of 4-(2-(4-fluorophenyl)-5-(1-methylpiperidin-4-yl)-1H-pyrrol-3-yl)pyridine (10) using benzyl 4-(5-(4-methoxyphenyl)-4-(pyridin-4-yl)-1H-pyrrol-2-yl)piperidine-1-carboxylate (43) (160 mgs, 0.343 mmol, 1 eq) and LAH 2M solution in THF (0.68 mL, 1.37 mmol, 4 eq), the title compound was synthesized. The crude product was dissolved in 1N HCl and partitioned between EtOAc and water. The organic phase was extracted with water (2×). The combined acidic aqueous phase was basified to pH 9 with 1N NaOH and EtOAc was added. The neutralised aqueous phase was extracted with EtOAc (3×). The combined organic phase was dried over sodium sulphate and concentrated. Purification by silica gel chromatography (2%→4%→6%→8%→10% MeOH/CHCl₃ with 0.25% NH₃) afforded the title compound Example 34 4-(2-(4-methoxyphenyl)-5-(1-methylpiperidin-4-yl)-1H-pyrrol-3-yl)pyridine (62 mgs, 53% yield, 97% purity by LC/MS) as a yellow solid. ¹H NMR (500 MHz, DMSO-4) δ 10.99 (d, J=2.7 Hz, 1H), 8.34-8.27 (m, 2H), 7.27-7.21 (m, 2H), 7.17-7.11 (m, 2H), 6.97-6.90 (m, 2H), 6.10 (dd, J=2.7, 0.8 Hz, 1H), 3.76 (s, 3H), 2.92-2.83 (m, 2H), 2.52 (s, 1H), 2.22 (s, 3H), 2.03 (s, 2H), 1.97-1.88 (m, 2H), 1.71-1.58 (in, 2H). (M+H)+ 347.35.

### Example 35. Synthesis of 4-(5-(1-methylpiperidin-4-yl)-2-(thiophen-2-yl)-1H-pyrrol-3-yl)pyridine

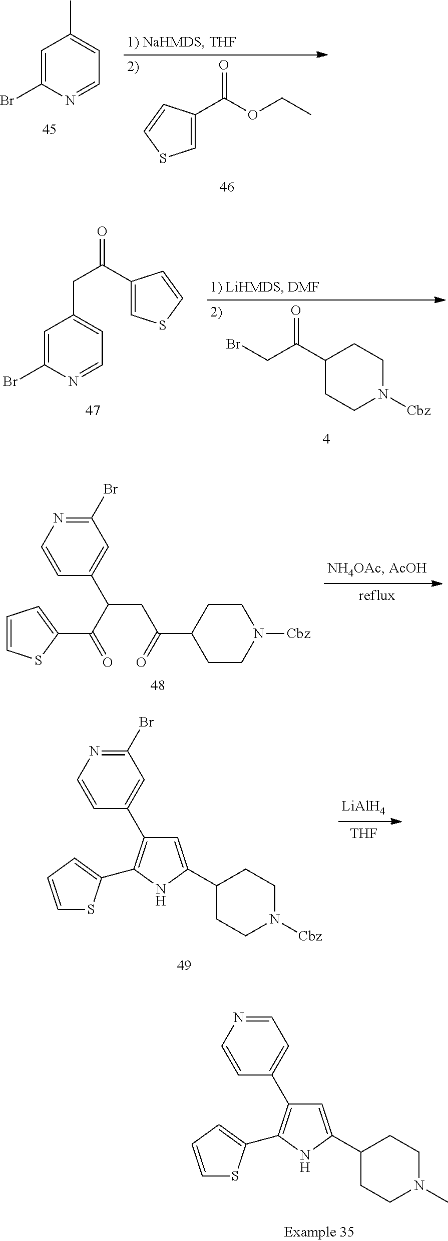

### a. 24(2-Bromopyridin-4-yl)-1-(thiophen-3-yl)ethan-1-one (47)

Following the procedure as described for the synthesis of 1-(4-fluorophenyl)-2-(pyridin-4-yl)ethan-1-one (7) using 2-bromo-4-methylpyridine (45) (1.032 g, 6 mmol, 1 eq), NaHMDS 1M solution in THF (6 mL, 6 mmol, 1 eq) and ethyl thiophene-3-carboxylate (46) (936 mgs, 6 mmol, 1 eq), the title compound (47) was synthesized. Purification by silica gel chromatography (10%→25%→50% EtOAc/Hexanes) afforded 2-(2-bromopyridin-4-yl)-1-(thiophen-3-yl)ethan-1-one (47) (300 mgs, 180% yield, 850 purity by LC/MS) (M+H)+ 281.65, 283.60.

### b. Benzyl 4-(3-(2-bromopyridin-4-yl)-4-oxo-4-(thiophen-2-yl)butanoyl)piperidine-1-carboxylate (48)

Following the procedure as described for the synthesis of benzyl 4-(4-(4-fluorophenyl)-4-oxo-3-(pyridin-4-yl)butanoyl)piperidine-1-carboxylate (8) using 2-(2-bromopyridin-4-yl)-1-(thiophen-3-yl)ethan-1-one (47) (228 mgs, 0.812 mmol, 1 eq), LiHMDS 1M solution in THF (0.98 mL, 0.98 mmol, 1.2 eq) and benzyl 4-(2-bromoacetyl)piperidine-1-carboxylate (4) (360 mgs, 1.056 mmol, 1.3 eq), the compound benzyl 4-(3-(2-bromopyridin-4-yl)-4-oxo-4-(thiophen-2-yl)butanoyl)piperidine-1-carboxylate (48) (596 mgs) was synthesized and taken onto the next step without any purification. (M+H)+ 540.85, 542.85.

### c. Benzyl 4-(4-(2-bromopyridin-4-yl)-5-(thiophen-2-yl)-1H-pyrrol-2-yl)piperidine-1-carboxylate (49)

Following the procedure as described for the synthesis of benzyl 4-(5-(4-fluorophenyl)-4-(pyridin-4-yl)-1H-pyrrol-2-yl)piperidine-1-carboxylate (9) using crude benzyl 4-(3-(2-bromopyridin-4-yl)-4-oxo-4-(thiophen-2-yl)butanoyl)piperidine-1-carboxylate (48) (596 mgs) and NH₄OAc (2 g), the compound (49) was synthesized. Purification by silica gel chromatography (10%→25%→50% EtOAc/Hexanes) afforded benzyl 4-(4-(2-bromopyridin-4-yl)-5-(thiophen-2-yl)-1H-pyrrol-2-yl)piperidine-1-carboxylate (49) (300 mgs, 89% purity by LC/MS) (M+H)+ 521.80, 523.80.

### d. 4-(5-(1-methylpiperidin-4-yl)-2-(thiophen-2-yl)-1H-pyrrol-3-yl)pyridine

Following the procedure as described for the synthesis of 4-(2-(4-fluorophenyl)-5-(1-methylpiperidin-4-yl)-1H-pyrrol-3-yl)pyridine (10) [TSP] using benzyl 4-(4-(2-bromopyridin-4-yl)-5-(thiophen-2-yl)-1H-pyrrol-2-yl)piperidine-1-carboxylate (49) (200 mgs, 0.384 mmol, 1 eq) and LAH 2M solution in THF (1 mL, 2.0 mmol, 5.2 eq), the title compound (50) was synthesized. The crude product was dissolved in 1N HCl and partitioned between EtOAc and water. The organic phase was extracted with water (2×). The combined acidic aqueous phase was basified to pH 9 with 1N NaOH and EtOAc was added. The neutralised aqueous phase was extracted with EtOAc (3×). The combined organic phase was dried over sodium sulphate and concentrated. Purification by silica gel chromatography (2%→4%→6%→8% MeOH/CHCl₃ with 0.25% NH₃) afforded the title compound Example 35 4-(5-(1-methylpiperidin-4-yl)-2-(thiophen-2-yl)-1H-pyrrol-3-yl)pyridine (82 mgs, 66% yield, 98% purity by LC/MS) as a yellow solid. ¹H NMR (500 MHz, DMSO-d₆) δ 11.05 (d, J=2.6 Hz, 1H), 8.38-8.30 (m, 2H), 7.55 (dd, J=5.0, 2.9 Hz, 1H), 7.44 (dd, J=3.0, 1.3 Hz, 1H), 7.24-7.16 (m, 2H), 7.02 (dd, J=5.0, 1.3 Hz, 1H), 6.10 (dd, J=2.7, 0.8 Hz, 1H), 2.87 (d, J=11.8 Hz, 2H), 2.54-2.50 (m, 1H), 2.22 (s, 3H), 2.03 (t, J=11.5 Hz, 2H), 1.96-1.87 (m, 2H), 1.65 (qd, J=12.7, 3.7 Hz, 2H), (M+H)+ 324.25.

### Example 36. Human PKG Assay

Full length PfPKG was cloned into the pTrcHisC vector for expression in BL21 (DE3) Star cells. An LB culture containing 50 ug/ml carbenicillin (250 mL) was grown overnight at 37° C. The following day the culture was cooled on ice for about 10-15 minutes and divided equally into two new 125 mL cultures. 125 ml of fresh LB broth containing carbenicillin was added to each flask bringing the volume of each flask to 250 ml. Cultures were shaken at 18° C. before induction with IPTG (1.0 mM) for 18-24 h. The bacterial pellet was frozen at −80° C. until protein purification. Cells were lysed with Bacterial Protein Extraction Reagent (Thermo Scientific) on ice for 10-15 min. Supernatant was obtained after centrifugation of lysate at 21,000×g for 15 min at 4° C. Supernatant was incubated for 30 minutes at 4° C. with rotation with HisPur Cobalt Resin (Thermo Scientific), pre-washed with Wash Buffer (50 mM Hepes pH 7.8, 120 mM KCl, 20 mM NaCl, 10 mM Imidazole). Resin was added to a gravity column that was allowed to empty through gravity flow. The resin was washed with Wash buffer until no more protein is eluted off the column as determined by Bradford assay. A second wash was performed with Wash buffer containing 300 mM NaCl as in the previous wash step. Bound protein was eluted in 50 mM Hepes pH 7.8, 120 mM KCl, 20 mM NaCl, 250 mM Imidazole. Protein was dialyzed overnight in 50 mM Hepes pH 7.8, 120 mM KCl, 20 mM NaCl and 10% glycerol, with two buffer exchanges. Dialyzed protein was concentrated in a Corning Spin-X concentrator and assayed for activity using the IMAP FP Progressive Screening Expression kit (Molecular Devices, catalog #R8127) following the manufacturer's protocol.

Human PKG, cloned into a pENTR221 vector, was obtained from DNASU.org. (HsCD00351766). It was cloned into a mammalian expression plasmid, Gateway pDEST27 (Invitrogen) for expression as a GST fusion protein, using LR Clonase II Plus enzyme (Invitrogen). The hPKG-pDEST27 plasmid was amplified in DH5a Max Efficiency Competent Cells and purified with a PureYield Plasmid Midiprep System (Promega) following the manufactures protocol. Expi293 cells were transfected with hPKG-pDEST27 using the Expifectamine 293 Transfection Kit (Gibco). After 18 hours, enhancing reagent (Gibco) was added. Cells were then collected 48 hours post-enhancing, centrifuged at 2000×g for 10 min, washed in ice cold PBS and frozen as a pellet at −80° C. until protein purification. The cell pellet was lysed with Mammalian Protein Extraction Reagent (Thermo Fisher) for 10-15 minutes. The lysate was centrifuged at 21,000×g for 15 minutes at 4° C. The supernatant was incubated for 1 h at 4° C. with glutathione resin that was pre-washed 10 times with ice cold Wash Buffer (50 mM Hepes pH 7.8, 120 mM KCl, 20 mM NaCl, 10% glycerol). The resin was loaded onto a column and washed with resin wash buffer by gravity flow until no more protein was detectable in the washes by Bradford assay. Protein was eluted with 10 mg/ml glutathione in Wash buffer. The protein was concentrated using a 30K MWCO Corning Spin X concentrator and assayed for activity as described above. Data for representative compounds of the invention is shown in Table 1 below.

### Example 37. PfPKG IMAP Assay

Plasmodium falciparum PKG (PfPKG) and human PKG (hPKG) kinase activity was assayed using a commercial immobilized metal ion affinity-based fluorescence polarization (IMAP) assay according to the manufactures protocol (Molecular Devices). Briefly, kinase assays (20 μl in black half volume 96 well microtiter plates) contained; 10 mM Tris-HCl, pH 7.2, 10 mM MgCl2, 0.05% NaN3, 0.01% Tween® 20, 10 uM ATP, 1 uM cGMP and 21 ng of recombinant enzyme per well. Compounds were preincubated with enzyme at 25° C. for 15 minutes and reactions were initiated with addition of 120 nM fluorescent peptide substrates, FAM-PKAtide for PfPKG and FAM-IP3R for hPKG (Molecular Devices). Fluorescent polarization was measured using a Synergy 2 Microplate reader (BioTek, Winooski, Vt.). Fluorescent polarization was read in parallel and perpendicular with an excitation wavelength of 485 nm and an emission wavelength of 528 nm. IC₅₀ data were analyzed using a four parameter logistic curve fit using Microsoft Excel Solver. Data for representative compounds of the invention is provided in Table 1 below.

TABLE 1

| | | Human PKG | PfPKG |
|---|---|---|---|
| Example | Structure | (10 μm) | (μm) |
| TSP | 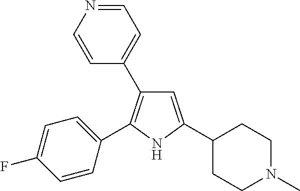 [SMILES: CN1CCC(c2cc(-c3ccncc3)c(-c3ccc(F)cc3)[nH]2)CC1] | 59 | <1 μm |
| 1 | 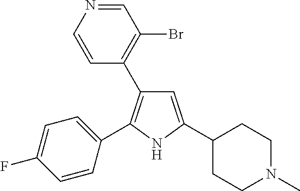 [SMILES: CN1CCC(c2cc(-c3ccncc3Br)c(-c3ccc(F)cc3)[nH]2)CC1] | 39 | <1 μm |
| 2 | 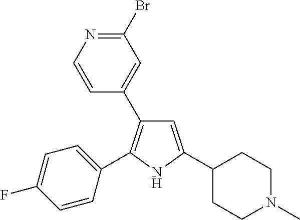 [SMILES: CN1CCC(c2cc(-c3ccnc(Br)c3)c(-c3ccc(F)cc3)[nH]2)CC1] | 60 | <1 μm |
| 3 | 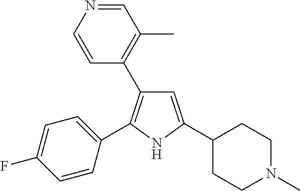 [SMILES: Cc1cnccc1-c1cc(C2CCN(C)CC2)[nH]c1-c1ccc(F)cc1] | 45 | <1 μm |
| 4 | 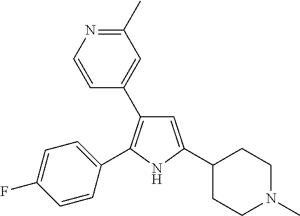 [SMILES: Cc1cc(-c2cc(C3CCN(C)CC3)[nH]c2-c2ccc(F)cc2)ccn1] | 30 | <1 μm |
| 5 | 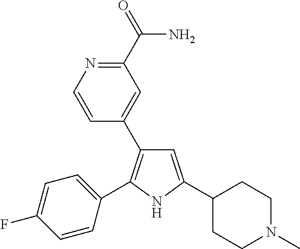 [SMILES: CN1CCC(c2cc(-c3ccnc(C(N)=O)c3)c(-c3ccc(F)cc3)[nH]2)CC1] | 28 | <1 μm |
| 6 | 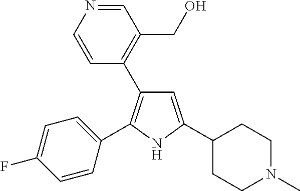 [SMILES: CN1CCC(c2cc(-c3ccncc3CO)c(-c3ccc(F)cc3)[nH]2)CC1] | −1 | <1 μm |
| 7 | 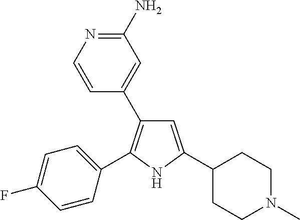 [SMILES: CN1CCC(c2cc(-c3ccnc(N)c3)c(-c3ccc(F)cc3)[nH]2)CC1] | 48 | <1 μm |
| 8 | 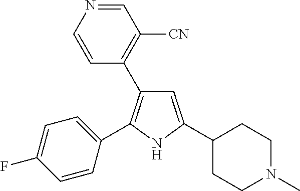 [SMILES: CN1CCC(c2cc(-c3ccncc3C#N)c(-c3ccc(F)cc3)[nH]2)CC1] | 12 | <1 μm |
| 9 | 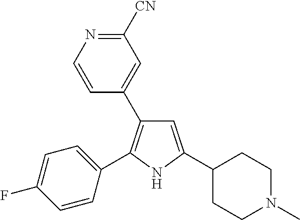 [SMILES: CN1CCC(c2cc(-c3ccnc(C#N)c3)c(-c3ccc(F)cc3)[nH]2)CC1] | 16 | <1 μm |
| 10 | 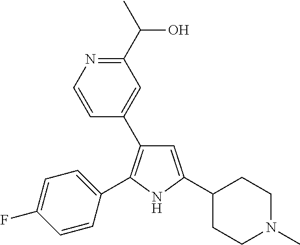 [SMILES: CC(O)c1cc(-c2cc(C3CCN(C)CC3)[nH]c2-c2ccc(F)cc2)ccn1] | −4 | <1 μm |
| 11 | 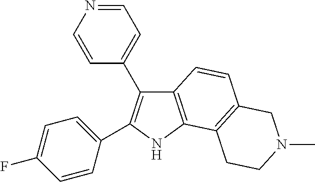 [SMILES: CN1CCc2c(ccc3c(-c4ccncc4)c(-c4ccc(F)cc4)[nH]c23)C1] | 35 | <1 μm |
| 12 | 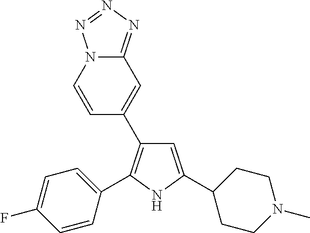 [SMILES: CN1CCC(c2cc(-c3ccn4nnnc4c3)c(-c3ccc(F)cc3)[nH]2)CC1] | −35 | <1 μm |
| 13 | 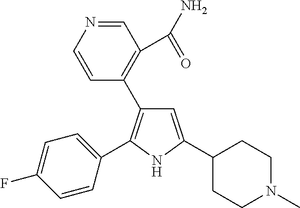 [SMILES: CN1CCC(c2cc(-c3ccncc3C(N)=O)c(-c3ccc(F)cc3)[nH]2)CC1] | −2 | <1 μm |
| 14 | 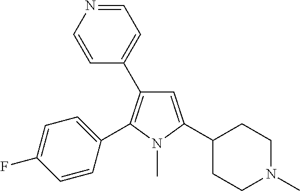 [SMILES: CN1CCC(c2cc(-c3ccncc3)c(-c3ccc(F)cc3)n2C)CC1] | 14 | <1 μm |
| 15 | 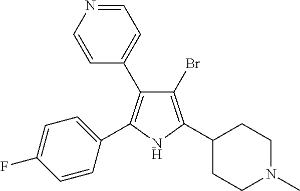 [SMILES: CN1CCC(c2[nH]c(-c3ccc(F)cc3)c(-c3ccncc3)c2Br)CC1] | 52 | <1 μm |
| 16 | 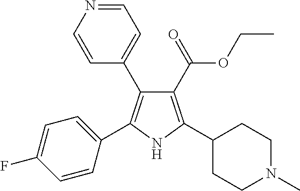 [SMILES: CCOC(=O)c1c(C2CCN(C)CC2)[nH]c(-c2ccc(F)cc2)c1-c1ccncc1] | 18 | <1 μm |
| 17 | 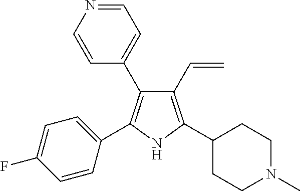 [SMILES: C=Cc1c(C2CCN(C)CC2)[nH]c(-c2ccc(F)cc2)c1-c1ccncc1] | 34 | <1 μm |
| 18 | 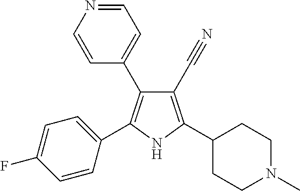 [SMILES: CN1CCC(c2[nH]c(-c3ccc(F)cc3)c(-c3ccncc3)c2C#N)CC1] | 57 | <1 μm |
| 19 | 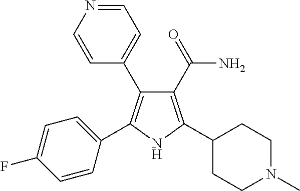 [SMILES: CN1CCC(c2[nH]c(-c3ccc(F)cc3)c(-c3ccncc3)c2C(N)=O)CC1] | −8 | <1 μm |
| 20 | 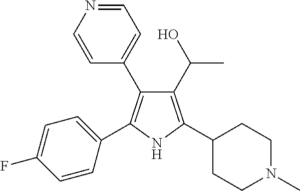 [SMILES: CC(O)c1c(C2CCN(C)CC2)[nH]c(-c2ccc(F)cc2)c1-c1ccncc1] | 3 | <1 μm |
| 21 | 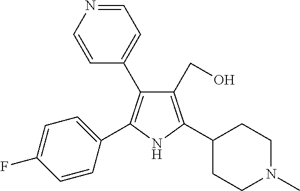 [SMILES: CN1CCC(c2[nH]c(-c3ccc(F)cc3)c(-c3ccncc3)c2CO)CC1] | 12 | <1 μm |
| 22 | 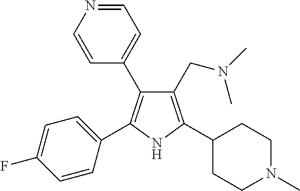 [SMILES: CN(C)Cc1c(C2CCN(C)CC2)[nH]c(-c2ccc(F)cc2)c1-c1ccncc1] | −3 | <1 μm |
| 23 | 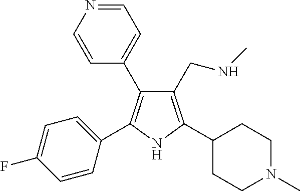 [SMILES: CNCc1c(C2CCN(C)CC2)[nH]c(-c2ccc(F)cc2)c1-c1ccncc1] | 0 | <1 μm |
| 24 | 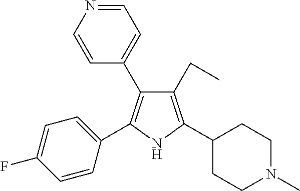 [SMILES: CCc1c(C2CCN(C)CC2)[nH]c(-c2ccc(F)cc2)c1-c1ccncc1] | 43 | <1 μm |
| 25 | 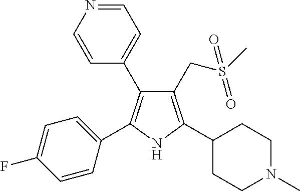 [SMILES: CN1CCC(c2[nH]c(-c3ccc(F)cc3)c(-c3ccncc3)c2CS(C)(=O)=O)CC1] | 17 | <1 μm |
| 26 | 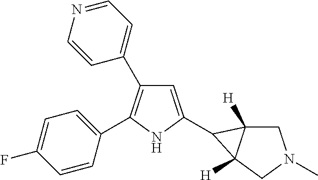 [SMILES: CN1C[C@@H]2C(c3cc(-c4ccncc4)c(-c4ccc(F)cc4)[nH]3)[C@@H]2C1] | 7 | <1 μm |
| 27 | 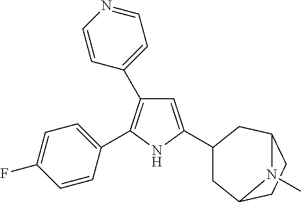 [SMILES: CN1C2CCC1CC(c1cc(-c3ccncc3)c(-c3ccc(F)cc3)[nH]1)C2] | 32 | <1 μm |
| 28 | 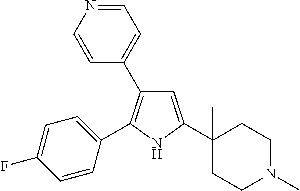 [SMILES: CN1CCC(C)(c2cc(-c3ccncc3)c(-c3ccc(F)cc3)[nH]2)CC1] | 29 | <1 μm |
| 29 | 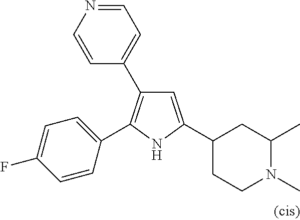 [SMILES: CC1CC(c2cc(-c3ccncc3)c(-c3ccc(F)cc3)[nH]2)CCN1C] | 26 | <1 μm |
| 30 | 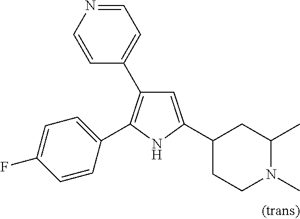 [SMILES: CC1CC(c2cc(-c3ccncc3)c(-c3ccc(F)cc3)[nH]2)CCN1C] | 5 | <1 μm |
| 31 | 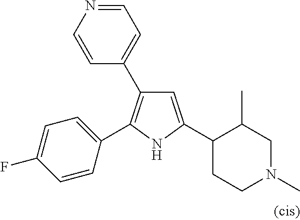 [SMILES: CC1CN(C)CCC1c1cc(-c2ccncc2)c(-c2ccc(F)cc2)[nH]1] | 82 | <1 μm |
| 32 | 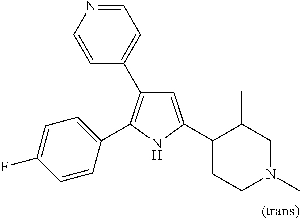 [SMILES: CC1CN(C)CCC1c1cc(-c2ccncc2)c(-c2ccc(F)cc2)[nH]1] | 66 | <1 μm |
| 33 | 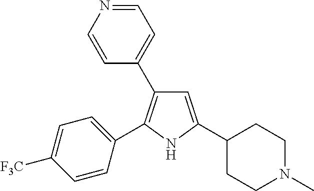 [SMILES: CN1CCC(c2cc(-c3ccncc3)c(-c3ccc(C(F)(F)F)cc3)[nH]2)CC1] | 9 | <1 μm |
| 34 | 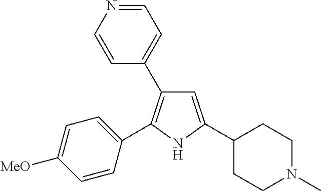 [SMILES: COc1ccc(-c2[nH]c(C3CCN(C)CC3)cc2-c2ccncc2)cc1] | 38 | <1 μm |
| 35 | 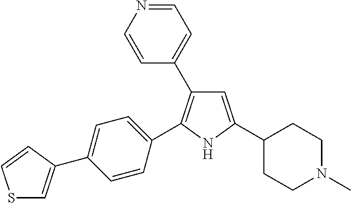 [SMILES: CN1CCC(c2cc(-c3ccncc3)c(-c3ccc(-c4ccsc4)cc3)[nH]2)CC1] | 66 | <1 μm |

### Example 38. Parasite Cell-Based EC₅₀ and Human Cell Cytotoxicity

Whole-cell activity was tested in the PbLuc sporozoite-HepG2 assay and in P. falciparum erythrocytic stage growth inhibition assays. Active compounds can be tested against P. falciparum sporozoites in HC04 assays (PMID: 30891005 PMCID: PMC6413710)

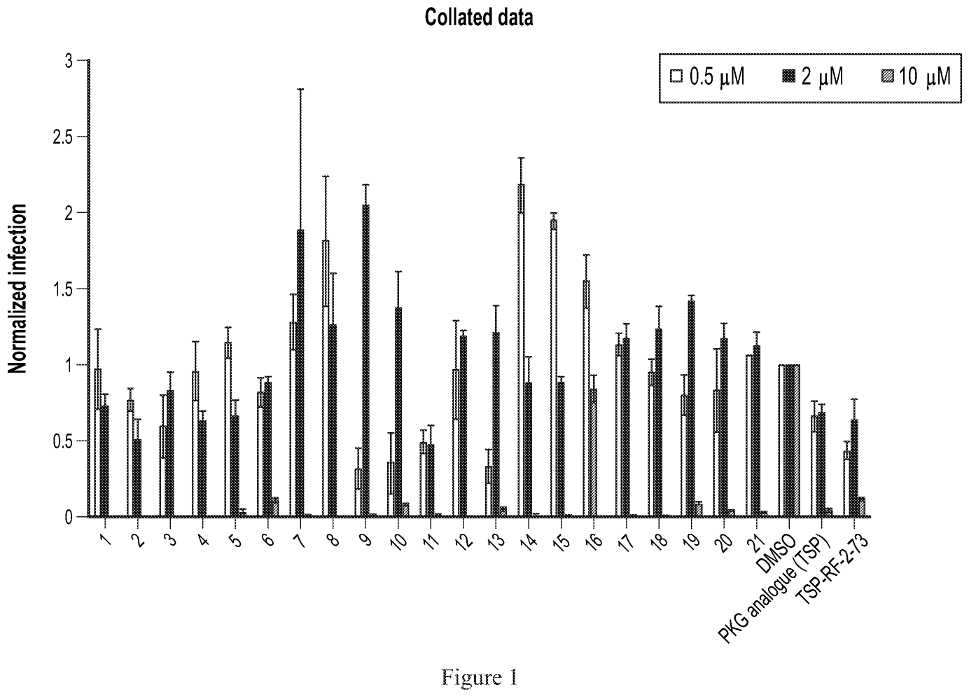

In the PbLuc-HepG2 assay, compounds were initially tested at 2 μM to screen for inhibition. Compounds and PbLuc sporozoites (10³/well) were added to HepG2 cells (1.6×10⁵/well) in triplicate. Vehicle and TSP were used as controls. Infected cells were quantified by luminescence measurements at 48 hours p.i. The total number of HepG2 cells in each well post-treatment was determined by colorimetric quantification (WST-1 assay, Sigma). For compounds demonstrating at least 50% inhibition of infection at this concentration, dose response curves can be established by testing a range of concentrations (10 nM-10 μM). EC₅₀ values will be calculated through nonlinear regression analysis of the dose-response curve. Alternatively, EC₅₀ from the luciferase-based assay can be verified by microscopic counting of immunostained liver stages, to eliminate test compounds with cross-reactivity against the firefly luciferase enzyme. Results for representative compounds of the invention are shown in FIG. 1.

### Example 39

The following illustrate representative pharmaceutical dosage forms, containing a compound of formula 1 (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X = (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 10 mg/ml) | mg/ml |
|---|---|
| Compound X = (free acid form) | 10.0 |
| Monobasic sodium phosphate | 0.3 |
| Dibasic sodium phosphate | 1.1 |
| Polyethylene glycol 400 | 200.0 |
| 1.0N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for treating malaria, reducing a human's susceptibility to malaria, or preventing malaria in a human comprising administering to the human, a compound of formula I: 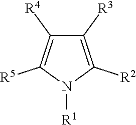
wherein:
R¹ is H or (C₁-C₃)alkyl;
R² is a 5-10 membered monocyclic or bicyclic heterocyclic ring comprising 1 or 2 nitrogen atoms, which 5-10 membered monocyclic or bicyclic heterocyclic ring is optionally substituted with 1, 2, 3, 4, or 5 substituents independently selected from the group consisting of (C₁-C₆)alkyl; and R³ is H, halo, hydroxy, cyano, NR^{a}R^{b}, —C(═O)NR^{a}R^{b}, (C₁-C₆)alkoxycarbonyl, (C₂-C₆)alkenyl, (C₂-C₆)alkynyl or (C₁-C₆)alkyl that is optionally substituted with one or more substituents independently selected from the group consisting of halo, hydroxy, cyano, NR^{a}R^{b}, (C₁-C₆)alkoxycarbonyl, —S(═O)₂—R^{e} and —C(═O)NR^{a}R^{b};
or
R² is a 5-10 membered monocyclic or bicyclic heterocyclic ring comprising 1 or 2 nitrogen atoms, which 5-10 membered monocyclic or bicyclic heterocyclic ring is optionally substituted with one or more substituents R^{x}; wherein a carbon atom of the R² 5-10 membered monocyclic or bicyclic heterocyclic ring adjacent to the position that attaches R² to the remainder of formula I, together with R³ forms a fused phenyl ring;
R⁴ is a tetrazolo[1,5-a]pyridine ring or a pyridine ring, which tetrazolo[1,5-a]pyridine ring or a pyridine ring is optionally substituted with 1, 2, 3, or 4 substituents independently selected from the group consisting of halo, hydroxy, cyano, NR^{c}R^{d}, —C(═O)NR^{c}R^{d} (C₁-C₆)alkoxycarbonyl, (C₂-C₆)alkenyl, (C₂-C₆)alkynyl, and (C₁-C₆)alkyl that is optionally substituted with one or more substituents independently selected from the group consisting of halo, hydroxy, cyano, NR^{c}R^{d}, (C₁-C₆)alkoxycarbonyl, and —C(═O)NR^{c}R^{d};
R⁵ is phenyl or thiophene, which phenyl or thiophene is optionally substituted with one or more substituents independently selected from the group consisting of halo, hydroxy, cyano, NR^{f}R^{g}, —C(═O)NR^{f}R^{g}, (C₁-C₆)alkoxycarbonyl, (C₁-C₆)alkoxy, (C₂-C₆)alkenyl, (C₂-C₆)alkynyl, thiophene, and (C₁-C₆)alkyl that is optionally substituted with one or more substituents independently selected from the group consisting of halo, hydroxy, cyano, NR^{f}R^{g}, (C₁-C₆)alkoxycarbonyl, and —C(═O)NR^{f}R^{g};
each R^{a} and R^{b} is independently selected from the group consisting of H, (C₁-C₆)alkyl, (C₃-C₆)cycloalkyl, and (C₃-C₆)cycloalkyl(C₁-C₆)alkyl; or R^{a} and R^{b} together with the nitrogen to which they are attached form a aziridino, azetidino, morpholino, piperazino, pyrrolidino or piperidino;
each R^{c} and R^{d} is independently selected from the group consisting of H, (C₁-C₆)alkyl, (C₃-C₆)cycloalkyl, and (C₃-C₆)cycloalkyl(C₁-C₆)alkyl; or R^{c} and R^{d} together with the nitrogen to which they are attached form a aziridino, azetidino, morpholino, piperazino, pyrrolidino or piperidino;
R^{e} is (C₁-C₆)alkyl;
each R^{f} and R^{g} is independently selected from the group consisting of H, (C₁-C₆)alkyl, (C₃-C₆)cycloalkyl, and (C₃-C₆)cycloalkyl(C₁-C₆)alkyl; or R^{f} and R^{g} together with the nitrogen to which they are attached form a aziridino, azetidino, morpholino, piperazino, pyrrolidino or piperidino; and
each R^{x} is independently selected from the group consisting of (C₁-C₆)alkyl, (C₂-C₆)alkenyl, (C₂-C₆)alkynyl, aryl, aryl(C₁-C₆)alkyl, (C₃-C₆)cycloalkyl, (C₁-C₆)alkoxycarbonyl, (C₁-C₆)alkanoyloxy, and (C₃-C₆)cycloalkyl(C₁-C₆)alkyl, wherein each R^{x} is optionally substituted with one or more groups independently selected from halo, cyano, nitro, (C₁-C₆)alkyl, and (C₁-C₆)alkoxy,
or a pharmaceutically acceptable salt thereof.

2. The method of claim 1, wherein R¹ is H and R⁵ is phenyl or thiophene, which phenyl or thiophene is optionally substituted with one or more substituents independently selected from the group consisting of halo, hydroxy, cyano, NR^{f}R^{g}, —C(═O)NR^{f}R^{g}, (C₁-C₆)alkoxycarbonyl, (C₁-C₆)alkoxy, (C₂-C₆)alkenyl, (C₂-C₆)alkynyl, and (C₁-C₆)alkyl that is optionally substituted with one or more substituents independently selected from the group consisting of halo, hydroxy, cyano, NR^{f}R^{g}, (C₁-C₆)alkoxycarbonyl, and —C(═O)NR^{f}R^{g}.

3. The method of claim 1, wherein:
R² is a 5-10 membered monocyclic or bicyclic heterocyclic ring comprising 1 or 2 nitrogen atoms, which 5-10 membered monocyclic or bicyclic heterocyclic ring is optionally substituted with one or more substituents R^{x}; and
R³ is halo, hydroxy, cyano, NR^{a}R^{b}, —C(═)NR^{a}R^{b}, (C₁-C₆)alkoxycarbonyl, (C₂-C₆)alkenyl, (C₂-C₆)alkynyl or (C₁-C₆)alkyl that is optionally substituted with one or more substituents independently selected from the group consisting of halo, hydroxy, cyano, NR^{a}R^{b}, (C₁-C₆)alkoxycarbonyl, and —C(═O)NR^{a}R^{b}.

4. The method of claim 3, wherein R² is piperidinyl, 3-azabicyclo[3.1.0]hexanyl, or 8-azabicyclo[3.2.1]octanyl, which R² is optionally substituted with 1, 2, 3, 4, or 5 substituents independently selected from the group consisting of (C₁-C₆)alkyl.

5. The method of claim 1, wherein R² is selected from the group consisting of:

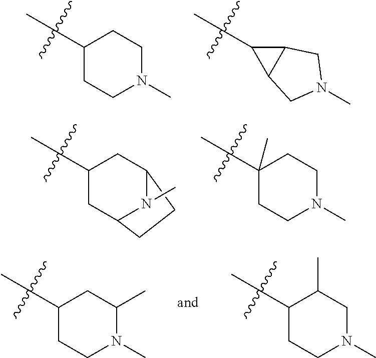

6. The method of claim 1, wherein R³ is H, halo, cyano, —C(═O)NR^{a}R^{b}, (C₁-C₆)alkoxycarbonyl, (C₂-C₆)alkenyl, or (C₁-C₆)alkyl that is optionally substituted with one or more substituents independently selected from the group consisting of hydroxy, NR^{a}R^{b}, and —S(═O)₂—R^{e}.

7. The method of claim 1, wherein R³ is H, bromo, aminocarbonyl, ethoxycarbonyl, vinyl, cyano, 1-hydroxyethyl, hydroxymethyl, N,N-dimethylaminomethyl, N-methylaminomethyl, ethyl, or methylsulfonylmethyl.

8. The method of claim 1, wherein the compound or pharmaceutically acceptable salt is a compound of formula (Ia): 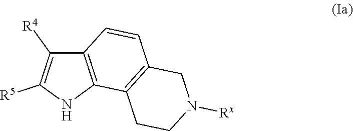
or a pharmaceutically acceptable salt thereof.

9. The method of claim 1, wherein R⁴ is a tetrazolo[1,5-a]pyridine ring.

10. The method of claim 1, wherein R⁴ is a pyridine ring that is optionally substituted with halo, cyano, NR^{c}R^{d}, —C(═O)NR^{c}R^{d}, or (C₁-C₆)alkyl that is optionally substituted with hydroxyl.

11. The method of claim 1, wherein R⁴ is a pyridine-4-yl, 3-bromopyridine-4-yl, 2-bromopyridine-4-yl, 3-methylpyridine-4-yl, 2-methylpyridine-4-yl, 2-(aminocarbonyl)pyridine-4-yl, 3-(hydroxymethyl)pyridine-4-yl, 2-aminopyridine-4-yl, 3-cyanopyridine-4-yl, 2-cyanopyridine-4-yl, 2-(1-hydroxyethyl)pyridine-4-yl, or 3-(aminocarbonyl)pyridine-4-yl.

12. The method of claim 1, wherein R⁵ is phenyl or thiophene, which phenyl or thiophene is optionally substituted with one or more substituents independently selected from the group consisting of halo, (C₁-C₆)alkoxy, and (C₁-C₆)alkyl that is optionally substituted with one or more substituents independently selected from the group consisting of halo.

13. The method of claim 1, wherein R⁵ is 4-fluorophenyl, 4-trifluoromethylphenyl, 4-methoxyphenyl, or 3-thiophene.

14. The method of claim 1, wherein the compound or pharmaceutically acceptable salt is a compound of formula (Ib): 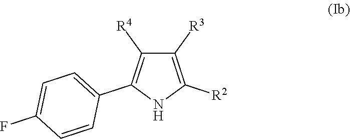
or a pharmaceutically acceptable salt thereof.

15. The method of claim 1, wherein the compound or pharmaceutically acceptable salt is selected from the group consisting of: 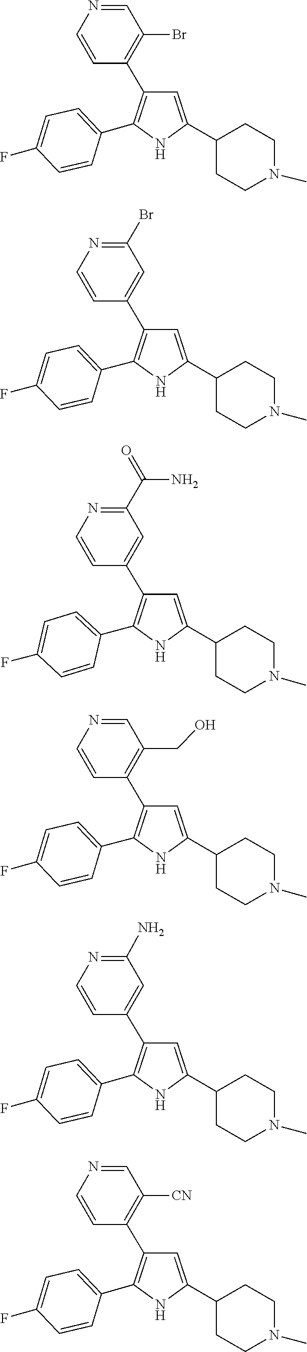 [SMILES: CN1CCC(c2cc(-c3ccncc3Br)c(-c3ccc(F)cc3)[nH]2)CC1 ; CN1CCC(c2cc(-c3ccnc(Br)c3)c(-c3ccc(F)cc3)[nH]2)CC1 ; CN1CCC(c2cc(-c3ccnc(C(N)=O)c3)c(-c3ccc(F)cc3)[nH]2)CC1 ; CN1CCC(c2cc(-c3ccncc3CO)c(-c3ccc(F)cc3)[nH]2)CC1 ; CN1CCC(c2cc(-c3ccnc(N)c3)c(-c3ccc(F)cc3)[nH]2)CC1 ; CN1CCC(c2cc(-c3ccncc3C#N)c(-c3ccc(F)cc3)[nH]2)CC1] 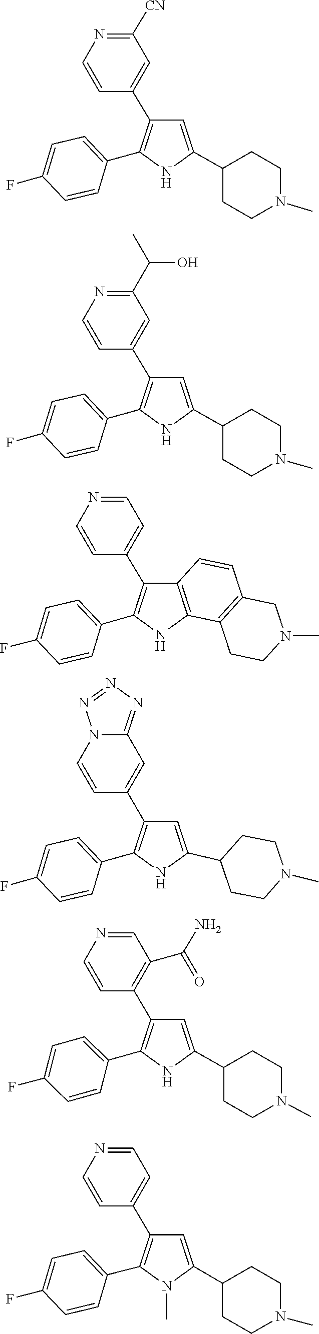 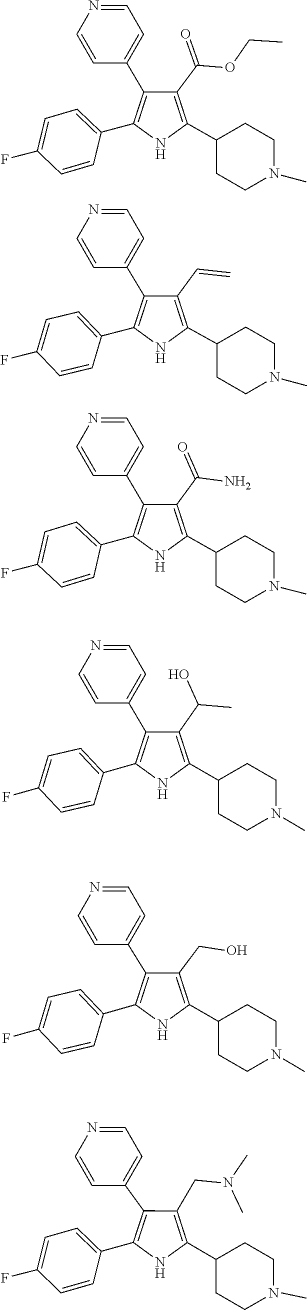 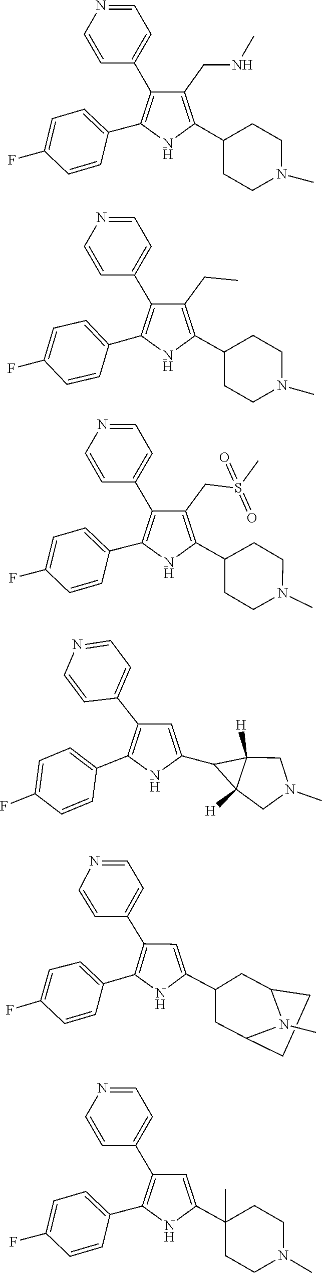 [SMILES: CNCc1c(C2CCN(C)CC2)[nH]c(-c2ccc(F)cc2)c1-c1ccncc1 ; CCc1c(C2CCN(C)CC2)[nH]c(-c2ccc(F)cc2)c1-c1ccncc1 ; CN1CCC(c2[nH]c(-c3ccc(F)cc3)c(-c3ccncc3)c2CS(C)(=O)=O)CC1 ; CN1C[C@@H]2C(c3cc(-c4ccncc4)c(-c4ccc(F)cc4)[nH]3)[C@@H]2C1 ; CN1C2CCC1CC(c1cc(-c3ccncc3)c(-c3ccc(F)cc3)[nH]1)C2 ; CN1CCC(C)(c2cc(-c3ccncc3)c(-c3ccc(F)cc3)[nH]2)CC1] 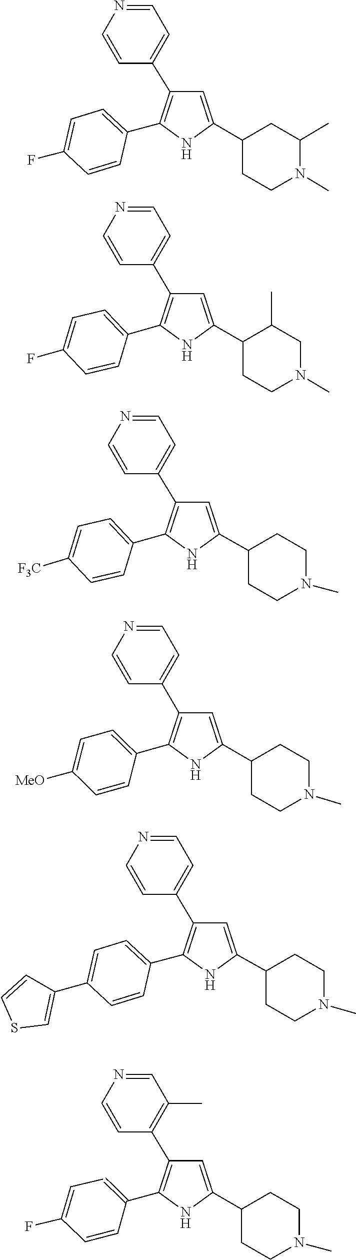 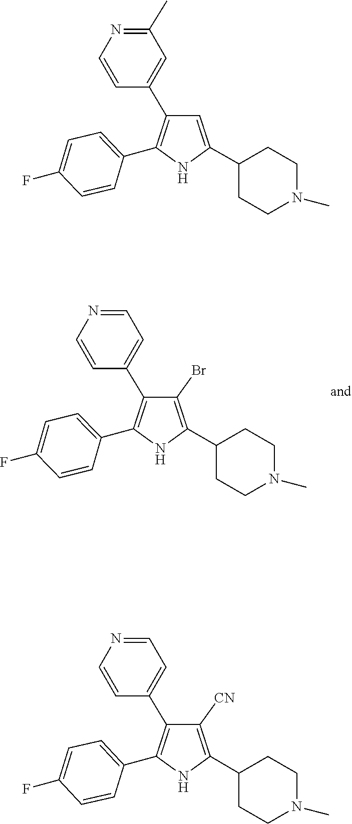 [SMILES: Cc1cc(-c2cc(C3CCN(C)CC3)[nH]c2-c2ccc(F)cc2)ccn1 ; CN1CCC(c2[nH]c(-c3ccc(F)cc3)c(-c3ccncc3)c2Br)CC1 ; CN1CCC(c2[nH]c(-c3ccc(F)cc3)c(-c3ccncc3)c2C#N)CC1]
and pharmaceutically acceptable salts thereof.

16. A pharmaceutical composition comprising a compound or pharmaceutically acceptable salt as described in claim 1 and a pharmaceutically acceptable excipient.

17-21. (canceled)

22. A compound of formula I: 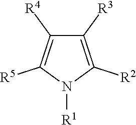
wherein:
R¹ is H or (C₁-C₃)alkyl;
R² is a 5-10 membered monocyclic or bicyclic heterocyclic ring comprising 1 or 2 nitrogen atoms, which 5-10 membered monocyclic or bicyclic heterocyclic ring is optionally substituted with 1, 2, 3, 4, or 5 substituents independently selected from the group consisting of (C₁-C₆)alkyl; and R³ is H, halo, hydroxy, cyano, NR^{a}R^{b}, —C(═O)NR^{a}R^{b}, (C₁-C₆)alkoxycarbonyl, (C₂-C₆)alkenyl, (C₂-C₆)alkynyl or (C₁-C₆)alkyl that is optionally substituted with one or more substituents independently selected from the group consisting of halo, hydroxy, cyano, NR^{a}R^{b}, (C₁-C₆)alkoxycarbonyl, —S(═O)₂—R^{e} and —C(═O)NR^{a}R^{b};
or
R² is a 5-10 membered monocyclic or bicyclic heterocyclic ring comprising 1 or 2 nitrogen atoms, which 5-10 membered monocyclic or bicyclic heterocyclic ring is optionally substituted with 1, 2, 3, 4, or 5 substituents independently selected from the group consisting of (C₁-C₆)alkyl, wherein a carbon atom of the R² 5-10 membered monocyclic or bicyclic heterocyclic ring adjacent to the position that attaches R² to the remainder of formula I, together with R³ forms a fused phenyl ring;
R⁴ is a tetrazolo[1,5-a]pyridine ring or a pyridine ring, which tetrazolo[1,5-a]pyridine ring or a pyridine ring is optionally substituted with 1, 2, 3, or 4 substituents independently selected from the group consisting of halo, hydroxy, cyano, NR^{c}R^{d}, —C(═O)NR^{c}R^{d} (C₁-C₆)alkoxycarbonyl, (C₂-C₆)alkenyl, (C₂-C₆)alkynyl, and (C₁-C₆)alkyl that is optionally substituted with one or more substituents independently selected from the group consisting of halo, hydroxy, cyano, NR^{c}R^{d}, (C₁-C₆)alkoxycarbonyl, and —C(═O)NR^{c}R^{d};
R⁵ is phenyl or thiophene, which phenyl or thiophene is optionally substituted with one or more substituents independently selected from the group consisting of halo, hydroxy, cyano, NR^{f}R^{g}, —C(═O)NR^{f}R^{g}, (C₁-C₆)alkoxycarbonyl, (C₁-C₆)alkoxy, (C₂-C₆)alkenyl, (C₂-C₆)alkynyl, thiophene, and (C₁-C₆)alkyl that is optionally substituted with one or more substituents independently selected from the group consisting of halo, hydroxy, cyano, NR^{f}R^{g}, (C₁-C₆)alkoxycarbonyl, and —C(═O)NR^{f}R^{g};
each R^{a} and R^{b} is independently selected from the group consisting of H, (C₁-C₆)alkyl, (C₃-C₆)cycloalkyl, and (C₃-C₆)cycloalkyl(C₁-C₆)alkyl; or R^{a} and R^{b} together with the nitrogen to which they are attached form a aziridino, azetidino, morpholino, piperazino, pyrrolidino or piperidino;
each R^{c} and R^{d} is independently selected from the group consisting of H, (C₁-C₆)alkyl, (C₃-C₆)cycloalkyl, and (C₃-C₆)cycloalkyl(C₁-C₆)alkyl; or R^{c} and R^{d} together with the nitrogen to which they are attached form a aziridino, azetidino, morpholino, piperazino, pyrrolidino or piperidino;
R^{e} is (C₁-C₆)alkyl; and
each R^{f} and R^{g} is independently selected from the group consisting of H, (C₁-C₆)alkyl, (C₃-C₆)cycloalkyl, and (C₃-C₆)cycloalkyl(C₁-C₆)alkyl; or R^{f} and R^{g} together with the nitrogen to which they are attached form a aziridino, azetidino, morpholino, piperazino, pyrrolidino or piperidino;
or a pharmaceutically acceptable salt thereof,
provided the compound is not:

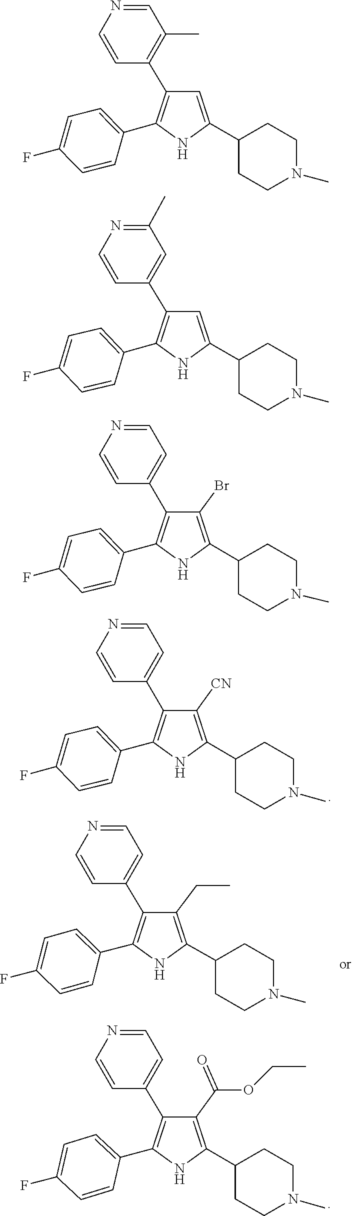

23. The compound or pharmaceutically acceptable salt of claim 22, wherein R¹ is H and R⁵ is phenyl or thiophene, which phenyl or thiophene is optionally substituted with one or more substituents independently selected from the group consisting of halo, hydroxy, cyano, NR^{f}R^{g}, —C(═O)NR^{f}R^{g}, (C₁-C₆)alkoxycarbonyl, (C₁-C₆)alkoxy, (C₂-C₆)alkenyl, (C₂-C₆)alkynyl, and (C₁-C₆)alkyl that is optionally substituted with one or more substituents independently selected from the group consisting of halo, hydroxy, cyano, NR^{f}R^{g}, (C₁-C₆)alkoxycarbonyl, and —C(═O)NR^{f}R^{g}.

24. The compound or pharmaceutically acceptable salt of claim 22 that is selected from the group consisting of: 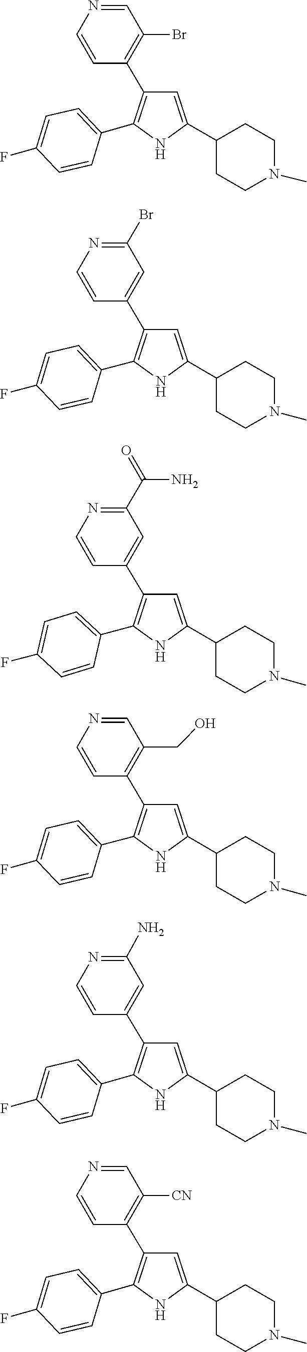 [SMILES: CN1CCC(c2cc(-c3ccncc3Br)c(-c3ccc(F)cc3)[nH]2)CC1 ; CN1CCC(c2cc(-c3ccnc(Br)c3)c(-c3ccc(F)cc3)[nH]2)CC1 ; CN1CCC(c2cc(-c3ccnc(C(N)=O)c3)c(-c3ccc(F)cc3)[nH]2)CC1 ; CN1CCC(c2cc(-c3ccncc3CO)c(-c3ccc(F)cc3)[nH]2)CC1 ; CN1CCC(c2cc(-c3ccnc(N)c3)c(-c3ccc(F)cc3)[nH]2)CC1 ; CN1CCC(c2cc(-c3ccncc3C#N)c(-c3ccc(F)cc3)[nH]2)CC1] 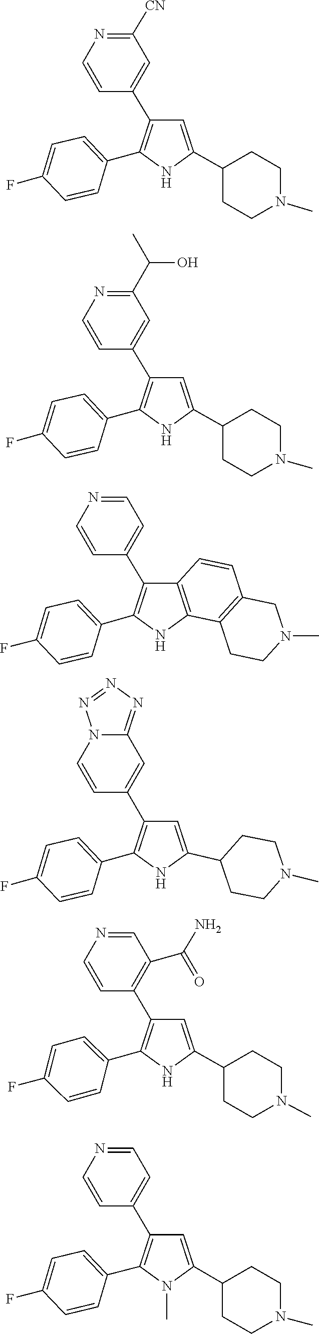 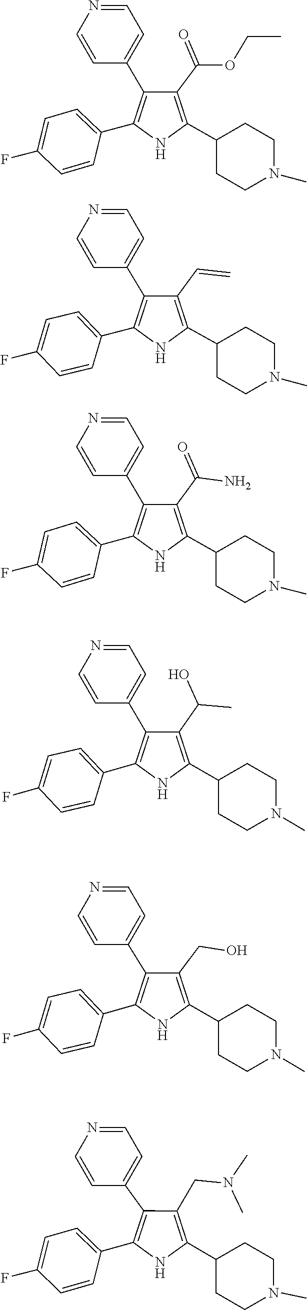 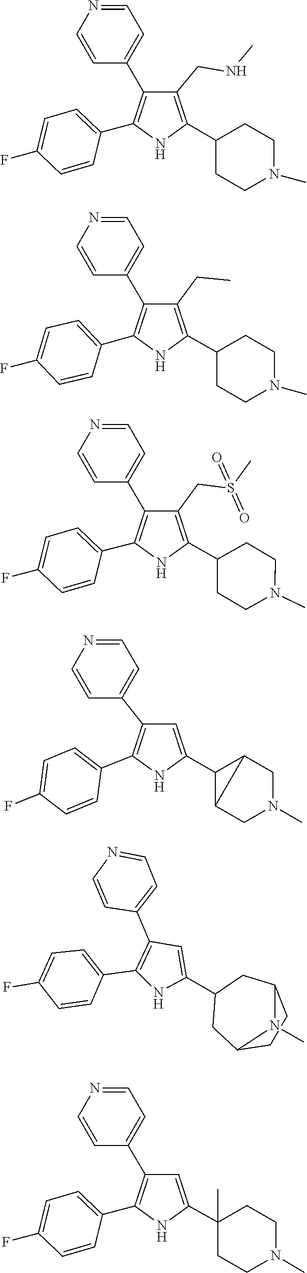 [SMILES: CNCc1c(C2CCN(C)CC2)[nH]c(-c2ccc(F)cc2)c1-c1ccncc1 ; CCc1c(C2CCN(C)CC2)[nH]c(-c2ccc(F)cc2)c1-c1ccncc1 ; CN1CCC(c2[nH]c(-c3ccc(F)cc3)c(-c3ccncc3)c2CS(C)(=O)=O)CC1 ; CN1CC2C(C1)C2c1cc(-c2ccncc2)c(-c2ccc(F)cc2)[nH]1 ; CN1C2CCC1CC(c1cc(-c3ccncc3)c(-c3ccc(F)cc3)[nH]1)C2 ; CN1CCC(C)(c2cc(-c3ccncc3)c(-c3ccc(F)cc3)[nH]2)CC1] 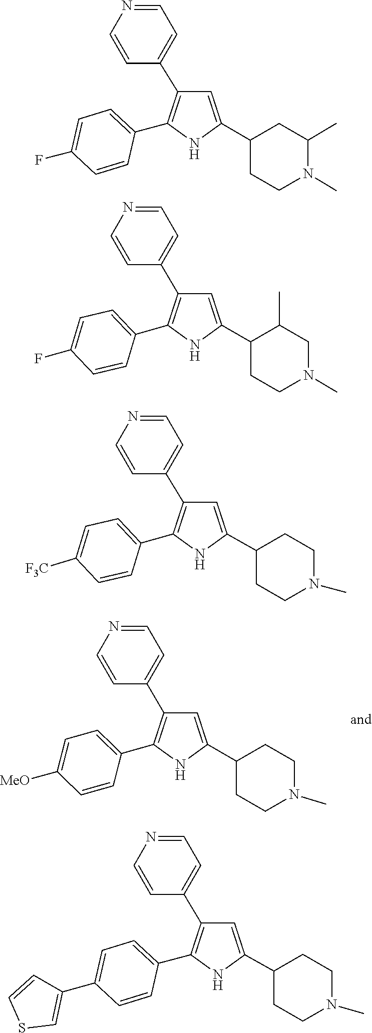
and pharmaceutically acceptable salts thereof.
